SEQUENCE LISTING (347 sequences)

SEQ ID NO: 1 - protein, 5 aa, synthetic construct
ELSMQ

SEQ ID NO: 2-6 - skipped (5 entries)

SEQ ID NO: 7 - protein, 17 aa, synthetic construct
GFDPDDGETMYAEKFQG

SEQ ID NO: 8 - protein, 17 aa, synthetic construct
GFDPDDGETMYAERFQG

SEQ ID NO: 9 - protein, 17 aa, synthetic construct
GFDPDGGETMYAEKFQG

SEQ ID NO: 10 - protein, 17 aa, synthetic construct
GFDPDDGETMYAEKLQG

SEQ ID NO: 11-17 - skipped (7 entries)

SEQ ID NO: 18 - protein, 9 aa, synthetic construct
SPGYDFFDY

SEQ ID NO: 19-25 - skipped (7 entries)

SEQ ID NO: 26 - protein, 11 aa, synthetic construct
GGNYIGSKSVH

SEQ ID NO: 27 - protein, 11 aa, synthetic construct
GGDNIGSKSVH

SEQ ID NO: 28 - protein, 11 aa, synthetic construct
GGNNIGSKSVH

SEQ ID NO: 29 - protein, 11 aa, synthetic construct
VGTNFGSKNVH

SEQ ID NO: 30 - protein, 14 aa, synthetic construct
TGTSSDVGGYNYVS

SEQ ID NO: 31 - protein, 11 aa, synthetic construct
GGNNIGSEGVH

SEQ ID NO: 32 - protein, 11 aa, synthetic construct
GGNNIGSKSVN

SEQ ID NO: 33 - protein, 11 aa, synthetic construct
GGNNIGSKSVF

SEQ ID NO: 34 - protein, 11 aa, synthetic construct
GGYNIGSKFVH

SEQ ID NO: 35 - protein, 11 aa, synthetic construct
GGNNTGSKSVH

SEQ ID NO: 36 - protein, 11 aa, synthetic construct
GGSDIGDEIVH

SEQ ID NO: 37 - protein, 11 aa, synthetic construct
GGSNIGDIRVH

SEQ ID NO: 38 - protein, 11 aa, synthetic construct
GGNNIGSKGVH

SEQ ID NO: 39 - protein, 11 aa, synthetic construct
GGNNIGSKNVH

SEQ ID NO: 40 - protein, 11 aa, synthetic construct
GGNNIGIKSVH

SEQ ID NO: 41 - protein, 11 aa, synthetic construct
GGNNIGRKAVH

SEQ ID NO: 42-52 - skipped (11 entries)

SEQ ID NO: 53 - protein, 7 aa, synthetic construct
DDGDRPS

SEQ ID NO: 54 - protein, 7 aa, synthetic construct
DDSDRPS

SEQ ID NO: 55 - protein, 7 aa, synthetic construct
EVSNRPS

SEQ ID NO: 56 - protein, 7 aa, synthetic construct
DDRDRPS

SEQ ID NO: 57 - protein, 7 aa, synthetic construct
DVSKRPS

SEQ ID NO: 58 - protein, 7 aa, synthetic construct
DDSDRPL

SEQ ID NO: 59-68 - skipped (10 entries)

SEQ ID NO: 69 - protein, 11 aa, synthetic construct
QVWDSISDHVV

SEQ ID NO: 70 - protein, 11 aa, synthetic construct
QVWDSSIDHLI

SEQ ID NO: 71 - protein, 11 aa, synthetic construct
QVWDSSNDLLI

SEQ ID NO: 72 - protein, 11 aa, synthetic construct
QVWDSSSDHLI

SEQ ID NO: 73 - protein, 11 aa, synthetic construct
QVWDSSSDHLV

SEQ ID NO: 74 - protein, 11 aa, synthetic construct
QVWDADSDQLI

SEQ ID NO: 75 - protein, 11 aa, synthetic construct
QVWDSSSDLLI

SEQ ID NO: 76-91 - skipped (16 entries)

SEQ ID NO: 92 - protein, 17 aa, synthetic construct
GFDPDAGETMYAEKFQG

SEQ ID NO: 93 - protein, 17 aa, synthetic construct
GFDPDSGETMYAEKFQG

SEQ ID NO: 94 - protein, 17 aa, synthetic construct
GFDPDEGETMYAEKFQG

SEQ ID NO: 95 - protein, 17 aa, synthetic construct
GFDPDDAETMYAEKFQG

SEQ ID NO: 96-101 - skipped (6 entries)

SEQ ID NO: 102 - protein, 17 aa, synthetic construct
  VARIANT 7: note = D or G
  VARIANT 14: note = K or R
  VARIANT 15: note = F or L
GFDPDDXETMYAEXXQG

SEQ ID NO: 103 - protein, 11 aa, synthetic construct
  VARIANT 3: note = N, D, or S
  VARIANT 4: note = Y, N, or D
  VARIANT 5: note = I or T
  VARIANT 7: note = S, D, I, R, or T
  VARIANT 8: note = K, E, or I
  VARIANT 9: note = S, I, R, G, N, or A
  VARIANT 11: note = H, F, or N
GGXXXGXXXVX

SEQ ID NO: 104 - protein, 7 aa, synthetic construct
  VARIANT 3: note = G, S, or R
  VARIANT 7: note = S or L
DDXDRPX

SEQ ID NO: 105 - protein, 11 aa, synthetic construct
  VARIANT 5: note = S or A
  VARIANT 6: note = I or S
  VARIANT 7: note = S, I, or N
  VARIANT 9: note = H, L, or Q
  VARIANT 10: note = V or L
  VARIANT 11: note = V or I
QVWDXXXDXXX

SEQ ID NO: 106 - protein, 7 aa, synthetic construct
  VARIANT 1: note = E or D
  VARIANT 4: note = N or K
XVSXRPS

SEQ ID NO: 107-117 - skipped (11 entries)

SEQ ID NO: 118 - protein, 11 aa, synthetic construct
GGNNIGTKSVH

SEQ ID NO: 119 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 447 aa, synthetic construct
EVQLVQSGAEVKKPGASVKVSCKVSGYTLTELSMQWVRQAPGKGLEWMGGFDPDDGETMYAERFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 447 aa, synthetic construct
QVQLVQSGAEVKKPGSSVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 447 aa, synthetic construct
EVRLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 447 aa, synthetic construct
EVQLVQSGAEVKKPGASVKVSCKVSGYTLTELSMQWVRQAPGKGLEWMGGFDPDGGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 447 aa, synthetic construct
QVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 447 aa, synthetic construct
EVQLVQSGAEVKKPGASVKVSCKVSGYTLTELSMQWVRQAPGKGLEWMGGFDPDDGETMYAEKFQGRLAVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 447 aa, synthetic construct
EVQLVQSGAEVKKPGASVKVSCKVSGYTLTELSMQWVRQAPGKGLEWMGGFDPDDGETMYAEKFQGRLTVTEDTPTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 447 aa, synthetic construct
EVQLVQSGAEVKKPGASVKVSCEVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 447 aa, synthetic construct
EVQLVQSGAEVKKPGASVKVSCKVSGYTLTELSMQWVRQAPGKGLEWMGGFDPDDGETMYAEKLQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129-141 - skipped (13 entries)

SEQ ID NO: 142 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 447 aa, synthetic construct
EVQLVQSGAEVKKPGASVKVSCKVSGYTLTELSMQWVRQAPGKGLEWMGGFDPDAGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 447 aa, synthetic construct
EVQLVQSGAEVKKPGASVKVSCKVSGYTLTELSMQWVRQAPGKGLEWMGGFDPDSGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 447 aa, synthetic construct
EVQLVQSGAEVKKPGASVKVSCKVSGYTLTELSMQWVRQAPGKGLEWMGGFDPDEGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - protein, 447 aa, synthetic construct
EVQLVQSGAEVKKPGASVKVSCKVSGYTLTELSMQWVRQAPGKGLEWMGGFDPDDAETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 447 aa, synthetic construct
EVQLVQSGAEVKKPGASVKVSCKVSGYTLTELSMQWVRQAPGKGLEWMGGFDPDAGETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 447 aa, synthetic construct
EVQLVQSGAEVKKPGASVKVSCKVSGYTLTELSMQWVRQAPGKGLEWMGGFDPDSGETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 150 - protein, 447 aa, synthetic construct
EVQLVQSGAEVKKPGASVKVSCKVSGYTLTELSMQWVRQAPGKGLEWMGGFDPDEGETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 151 - protein, 447 aa, synthetic construct
EVQLVQSGAEVKKPGASVKVSCKVSGYTLTELSMQWVRQAPGKGLEWMGGFDPDDAETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 447 aa, synthetic construct
QVQLVQSGAEVKKPGASVKVSCKVSGYTLTELSMQWVRQAPGKGLEWMGGFDPDDGETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 447 aa, synthetic construct
QVQLVQSGAEVKKPGASVKVSCKVSGYTLTELSMQWVRQAPGKGLEWMGGFDPDAGETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 154 - protein, 447 aa, synthetic construct
QVQLVQSGAEVKKPGASVKVSCKVSGYTLTELSMQWVRQAPGKGLEWMGGFDPDSGETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 155 - protein, 447 aa, synthetic construct
QVQLVQSGAEVKKPGASVKVSCKVSGYTLTELSMQWVRQAPGKGLEWMGGFDPDEGETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 447 aa, synthetic construct
QVQLVQSGAEVKKPGASVKVSCKVSGYTLTELSMQWVRQAPGKGLEWMGGFDPDDAETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 157 - protein, 447 aa, synthetic construct
QVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 158 - protein, 447 aa, synthetic construct
EVQLVQSGAEVKKPGASVKVSCKVSGYTLTELSMQWVRQAPGKGLEWMGGFDPDDGETMYAEKFQGRVTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 447 aa, synthetic construct
EVQLVQSGAEVKKPGASVKVSCKVSGYTLTELSMQWVRQAPGKGLEWMGGFDPDDGETMYAEKFQGRLTMTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTAYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELSSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLRSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 166 - protein, 118 aa, synthetic construct
EVQLVQSGAEVKKPGASVKVSCKVSGYTLTELSMQWVRQAPGKGLEWMGGFDPDDGETMYAERFQGRLTVTEDTSTDTVYMELRSLTSEDTALYFCATSPGYDFFDYWGQGTLVTVSS

SEQ ID NO: 167 - protein, 118 aa, synthetic construct
QVQLVQSGAEVKKPGSSVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 168 - protein, 118 aa, synthetic construct
EVRLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 169 - protein, 118 aa, synthetic construct
EVQLVQSGAEVKKPGASVKVSCKVSGYTLTELSMQWVRQAPGKGLEWMGGFDPDGGETMYAEKFQGRLTVTEDTSTDTVYMELRSLTSEDTALYFCATSPGYDFFDYWGQGTLVTVSS

SEQ ID NO: 170 - protein, 118 aa, synthetic construct
QVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 171 - protein, 118 aa, synthetic construct
EVQLVQSGAEVKKPGASVKVSCKVSGYTLTELSMQWVRQAPGKGLEWMGGFDPDDGETMYAEKFQGRLAVTEDTSTDTVYMELRSLTSEDTALYFCATSPGYDFFDYWGQGTLVTVSS

SEQ ID NO: 172 - protein, 118 aa, synthetic construct
EVQLVQSGAEVKKPGASVKVSCKVSGYTLTELSMQWVRQAPGKGLEWMGGFDPDDGETMYAEKFQGRLTVTEDTPTDTVYMELRSLTSEDTALYFCATSPGYDFFDYWGQGTLVTVSS

SEQ ID NO: 173 - protein, 118 aa, synthetic construct
EVQLVQSGAEVKKPGASVKVSCEVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 174 - protein, 118 aa, synthetic construct
EVQLVQSGAEVKKPGASVKVSCKVSGYTLTELSMQWVRQAPGKGLEWMGGFDPDDGETMYAEKLQGRLTVTEDTSTDTVYMELRSLTSEDTALYFCATSPGYDFFDYWGQGTLVTVSS

SEQ ID NO: 175-187 - skipped (13 entries)

SEQ ID NO: 188 - protein, 118 aa, synthetic construct
EVQLVQSGAEVKKPGASVKVSCKVSGYTLTELSMQWVRQAPGKGLEWMGGFDPDAGETMYAEKFQGRLTVTEDTSTDTVYMELRSLTSEDTALYFCATSPGYDFFDYWGQGTLVTVSS

SEQ ID NO: 189 - protein, 118 aa, synthetic construct
EVQLVQSGAEVKKPGASVKVSCKVSGYTLTELSMQWVRQAPGKGLEWMGGFDPDSGETMYAEKFQGRLTVTEDTSTDTVYMELRSLTSEDTALYFCATSPGYDFFDYWGQGTLVTVSS

SEQ ID NO: 190 - protein, 118 aa, synthetic construct
EVQLVQSGAEVKKPGASVKVSCKVSGYTLTELSMQWVRQAPGKGLEWMGGFDPDEGETMYAEKFQGRLTVTEDTSTDTVYMELRSLTSEDTALYFCATSPGYDFFDYWGQGTLVTVSS

SEQ ID NO: 191 - protein, 118 aa, synthetic construct
EVQLVQSGAEVKKPGASVKVSCKVSGYTLTELSMQWVRQAPGKGLEWMGGFDPDDAETMYAEKFQGRLTVTEDTSTDTVYMELRSLTSEDTALYFCATSPGYDFFDYWGQGTLVTVSS

SEQ ID NO: 192 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELSSLRSEDTAVYFCATSPGYDFFDYWGQGTLVTVSS

SEQ ID NO: 193 - protein, 118 aa, synthetic construct
EVQLVQSGAEVKKPGASVKVSCKVSGYTLTELSMQWVRQAPGKGLEWMGGFDPDAGETMYAEKFQGRLTVTEDTSTDTVYMELSSLRSEDTAVYFCATSPGYDFFDYWGQGTLVTVSS

SEQ ID NO: 194 - protein, 118 aa, synthetic construct
EVQLVQSGAEVKKPGASVKVSCKVSGYTLTELSMQWVRQAPGKGLEWMGGFDPDSGETMYAEKFQGRLTVTEDTSTDTVYMELSSLRSEDTAVYFCATSPGYDFFDYWGQGTLVTVSS

SEQ ID NO: 195 - protein, 118 aa, synthetic construct
EVQLVQSGAEVKKPGASVKVSCKVSGYTLTELSMQWVRQAPGKGLEWMGGFDPDEGETMYAEKFQGRLTVTEDTSTDTVYMELSSLRSEDTAVYFCATSPGYDFFDYWGQGTLVTVSS

SEQ ID NO: 196 - protein, 118 aa, synthetic construct
EVQLVQSGAEVKKPGASVKVSCKVSGYTLTELSMQWVRQAPGKGLEWMGGFDPDDAETMYAEKFQGRLTVTEDTSTDTVYMELSSLRSEDTAVYFCATSPGYDFFDYWGQGTLVTVSS

SEQ ID NO: 197 - protein, 118 aa, synthetic construct
QVQLVQSGAEVKKPGASVKVSCKVSGYTLTELSMQWVRQAPGKGLEWMGGFDPDDGETMYAEKFQGRVTMTEDTSTDTAYMELSSLRSEDTAVYYCATSPGYDFFDYWGQGTLVTVSS

SEQ ID NO: 198 - protein, 118 aa, synthetic construct
QVQLVQSGAEVKKPGASVKVSCKVSGYTLTELSMQWVRQAPGKGLEWMGGFDPDAGETMYAEKFQGRVTMTEDTSTDTAYMELSSLRSEDTAVYYCATSPGYDFFDYWGQGTLVTVSS

SEQ ID NO: 199 - protein, 118 aa, synthetic construct
QVQLVQSGAEVKKPGASVKVSCKVSGYTLTELSMQWVRQAPGKGLEWMGGFDPDSGETMYAEKFQGRVTMTEDTSTDTAYMELSSLRSEDTAVYYCATSPGYDFFDYWGQGTLVTVSS

SEQ ID NO: 200 - protein, 118 aa, synthetic construct
QVQLVQSGAEVKKPGASVKVSCKVSGYTLTELSMQWVRQAPGKGLEWMGGFDPDEGETMYAEKFQGRVTMTEDTSTDTAYMELSSLRSEDTAVYYCATSPGYDFFDYWGQGTLVTVSS

SEQ ID NO: 201 - protein, 118 aa, synthetic construct
QVQLVQSGAEVKKPGASVKVSCKVSGYTLTELSMQWVRQAPGKGLEWMGGFDPDDAETMYAEKFQGRVTMTEDTSTDTAYMELSSLRSEDTAVYYCATSPGYDFFDYWGQGTLVTVSS

SEQ ID NO: 202 - protein, 118 aa, synthetic construct
EVQLVQSGAEVKKPGASVKVSCKVSGYTLTELSMQWVRQAPGKGLEWMGGFDPDDGETMYAEKFQGRVTVTEDTSTDTVYMELRSLTSEDTALYFCATSPGYDFFDYWGQGTLVTVSS

SEQ ID NO: 203 - protein, 118 aa, synthetic construct
EVQLVQSGAEVKKPGASVKVSCKVSGYTLTELSMQWVRQAPGKGLEWMGGFDPDDGETMYAEKFQGRLTMTEDTSTDTVYMELRSLTSEDTALYFCATSPGYDFFDYWGQGTLVTVSS

SEQ ID NO: 204 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTAYMELRSLTSEDTALYFCATSPGYDFFDYWGQGTLVTVSS

SEQ ID NO: 205 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELSSLTSEDTALYFCATSPGYDFFDYWGQGTLVTVSS

SEQ ID NO: 206 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLRSEDTALYFCATSPGYDFFDYWGQGTLVTVSS

SEQ ID NO: 207 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTAVYFCATSPGYDFFDYWGQGTLVTVSS

SEQ ID NO: 208 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYYCATSPGYDFFDYWGQGTLVTVSS

SEQ ID NO: 209 - protein, 214 aa, synthetic construct
SYVLTQPPSVSVAPGKTARITCGGNYIGSKSVHWYQQKPGQAPVLVVYDDGDRPSGIPARFSGSNSGNTATLTISRVEAGDEADYYCQVWDSISDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 210 - protein, 214 aa, synthetic construct
SYELTQPPSVSVAPGKTARITCGGDNIGSKSVHWYQQKPGQAPVLVVYDDSDRPSGIPERFSGSNSGNTATLTISRVEAGDEADYYCQVWDSSIDHLIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 211 - protein, 214 aa, synthetic construct
SYVLTQPPSVSVAPGQTARITCGGNNIGSKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 212 - protein, 214 aa, synthetic construct
SYVLTQPPSVSVAPGQTASITCVGTNFGSKNVHWYQQRPGQAPVLVVYDDSDRPSGIPERFSGSNSGNTATLTISRVEAGDEADYYCQVWDSSSDHLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 213 - protein, 217 aa, synthetic construct
QSALTQPASVSGSPGQSITISCTGTSSDVGGYNYVSWYQQHPGKAPKLMIYEVSNRPSGVSNRFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 214 - protein, 214 aa, synthetic construct
SYVLTQPPSVSVAPGQTASITCVGTNFGSKNVHWYQQRPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 215 - protein, 214 aa, synthetic construct
SYELTQPPSVSVAPGQTARITCGGNNIGSKSVHWYQQKPGQAPVLVVYDDSDRPSGIPERFSGSNSGRTATLTISRVEAGDEADYHCQVWDSSSDHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 216 - protein, 214 aa, synthetic construct
SYVLTQPPSVSVAPGQTARIPCGGNNIGSKSVHWYQQKPGQAPVLVVYDDRDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 217 - protein, 214 aa, synthetic construct
SYELTQPPSVSVAPGQTARIPCGGNNIGSEGVHWYQQKPGQAPVLVVYDDSDRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 218 - protein, 217 aa, synthetic construct
QSALTQPRSVSGSPGQSVTISCTGTSSDVGGYNYVSWYQQHPGKAPKLMIYDVSKRPSGVPDRFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 219 - protein, 214 aa, synthetic construct
SYVLTQPPSVSVAPGQTARVSCGGNNIGSKSVNWYRQKPGQAPVLVIYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 220 - protein, 214 aa, synthetic construct
SYVLTQPPSVSVAPGKTARITCGGNNIGSKSVFWYQQKPGQAPVLVVYDDSDRPSGIPERFSGSNSGNTATLTISGTQAMDEADYYCQVWDSSSDHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 221 - protein, 214 aa, synthetic construct
SYVLTQPPSVSVAPGKTARITCGGYNIGSKFVHWYRQRPGQAPVLVVYDDRDRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 222 - protein, 214 aa, synthetic construct
SYELTQPPSVSVAPGKTARITCGGDNIGSKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 223 - protein, 214 aa, synthetic construct
SYVLTQPPSVSVAPGKTARITCGGNNTGSKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 224 - protein, 214 aa, synthetic construct
SYVLTQPPSVSVAPGQTARITCGGSDIGDEIVHWYQQKPGQAPVLVVYDDSDRPSGIPERFSGSNSGNTATLTISRAQAGDEADYYCQVWDSSSDHLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 225 - protein, 214 aa, synthetic construct
SYVLTQPPSVSVAPGKTARITCGGNNIGSKSVHWYQQKPGQAPVLVVYDDSDRPLGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 226 - protein, 214 aa, synthetic construct
SYVLTQPLSVSVALGQTARITCGGSNIGDIRVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 227 - protein, 214 aa, synthetic construct
SYVLTQPPSVSVAPGKTARITCGGNNIGSKGVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 228 - protein, 214 aa, synthetic construct
SYVLTQPPSVSVAPGQTARVSCGGNNIGSKSVNWYRQKPGQAPVLVIYDDSDRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 229 - protein, 214 aa, synthetic construct
SYVLTQPPSVSVAPGKTARITCGGNNIGSKNVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 230 - protein, 214 aa, synthetic construct
SYVLTQPPSVSVAPGQTARITCGGNNIGIKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 231 - protein, 214 aa, synthetic construct
SYVLTQPPSVSVAPGKTARITCGGNNIGSKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 232 - protein, 214 aa, synthetic construct
SYELTQPPSVAVAPGQTARITCGGNNIGRKAVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 233 - protein, 214 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSIDHLIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 234 - protein, 214 aa, synthetic construct
SYVLTQPPSVSVAPGQTARITCGGNNIGIKSVHWYQQKPGQAPVLVVYDDRDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 235 - protein, 214 aa, synthetic construct
SYELTQPPSVSVAPGQTARITCGGNNIGSKSVHWYQQKPGQAPVLVVYDDSDRPSGIPERFSGSNSGNTATLTISRVEAGDEADYYCQVWDADSDQLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 236 - protein, 214 aa, synthetic construct
SYVLTQPPSVSVAPGKTARITCGGNNIGSKSVHWYQQKPGQAPVLVVYDDSDRPSGIPERFSGSNSGNTATLTISRVEAGDEADYYCQVWDSSSDLLIFGGGAKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 237 - protein, 214 aa, synthetic construct
SYVLTQPPSVSVAPGKTATITCGGNNIGTKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 238-259 - skipped (22 entries)

SEQ ID NO: 260 - protein, 217 aa, synthetic construct
SYVLTQPPSVSVAPGKTARITCTGTSSDVGGYNYVSWYQQKPGQAPVLVVYEVSNRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 261 - protein, 214 aa, synthetic construct
SYVLTQPPSVSVAPGKTARITCGGNNIGSKSVHWYQQKPGQAPVLVVYDDSDRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 262-263 - skipped (2 entries)

SEQ ID NO: 264 - protein, 108 aa, synthetic construct
SYVLTQPPSVSVAPGKTARITCGGNYIGSKSVHWYQQKPGQAPVLVVYDDGDRPSGIPARFSGSNSGNTATLTISRVEAGDEADYYCQVWDSISDHVVFGGGTKLTVL

SEQ ID NO: 265 - protein, 108 aa, synthetic construct
SYELTQPPSVSVAPGKTARITCGGDNIGSKSVHWYQQKPGQAPVLVVYDDSDRPSGIPERFSGSNSGNTATLTISRVEAGDEADYYCQVWDSSIDHLIFGGGTKVTVL

SEQ ID NO: 266 - protein, 108 aa, synthetic construct
SYVLTQPPSVSVAPGQTARITCGGNNIGSKSVHWYQQKPGQAPVLVVYDDSDRPSGIPERFSGSNSGNTATLTINRVEAGDEADYYCQVWDSSNDLLIFGGGTKLTVL

SEQ ID NO: 267 - protein, 108 aa, synthetic construct
SYVLTQPPSVSVAPGQTASITCVGTNFGSKNVHWYQQRPGQAPVLVVYDDSDRPSGIPERFSGSNSGNTATLTISRVEAGDEADYYCQVWDSSSDHLIFGGGTKLTVL

SEQ ID NO: 268 - protein, 111 aa, synthetic construct
QSALTQPASVSGSPGQSITISCTGTSSDVGGYNYVSWYQQHPGKAPKLMIYEVSNRPSGVSNRFSGSNSGNTATLTINRVEAGDEADYYCQVWDSSNDLLIFGGGTKLTVL

SEQ ID NO: 269 - protein, 108 aa, synthetic construct
SYVLTQPPSVSVAPGQTASITCVGTNFGSKNVHWYQQRPGQAPVLVVYDDSDRPSGIPERFSGSNSGNTATLTINRVEAGDEADYYCQVWDSSNDLLIFGGGTKLTVL

SEQ ID NO: 270 - protein, 108 aa, synthetic construct
SYELTQPPSVSVAPGQTARITCGGNNIGSKSVHWYQQKPGQAPVLVVYDDSDRPSGIPERFSGSNSGRTATLTISRVEAGDEADYHCQVWDSSSDHLVFGGGTKLTVL

SEQ ID NO: 271 - protein, 108 aa, synthetic construct
SYVLTQPPSVSVAPGQTARIPCGGNNIGSKSVHWYQQKPGQAPVLVVYDDRDRPSGIPERFSGSNSGNTATLTINRVEAGDEADYYCQVWDSSNDLLIFGGGTKLTVL

SEQ ID NO: 272 - protein, 108 aa, synthetic construct
SYELTQPPSVSVAPGQTARIPCGGNNIGSEGVHWYQQKPGQAPVLVVYDDSDRPSGIPERFSGSNSGNTATLTISRVEAGDEADYYCQVWDSSNDLLIFGGGTKLTVL

SEQ ID NO: 273 - protein, 111 aa, synthetic construct
QSALTQPRSVSGSPGQSVTISCTGTSSDVGGYNYVSWYQQHPGKAPKLMIYDVSKRPSGVPDRFSGSNSGNTATLTINRVEAGDEADYYCQVWDSSNDLLIFGGGTKLTVL

SEQ ID NO: 274 - protein, 108 aa, synthetic construct
SYVLTQPPSVSVAPGQTARVSCGGNNIGSKSVNWYRQKPGQAPVLVIYDDSDRPSGIPERFSGSNSGNTATLTINRVEAGDEADYYCQVWDSSNDLLIFGGGTKLTVL

SEQ ID NO: 275 - protein, 108 aa, synthetic construct
SYVLTQPPSVSVAPGKTARITCGGNNIGSKSVFWYQQKPGQAPVLVVYDDSDRPSGIPERFSGSNSGNTATLTISGTQAMDEADYYCQVWDSSSDHLVFGGGTKLTVL

SEQ ID NO: 276 - protein, 108 aa, synthetic construct
SYVLTQPPSVSVAPGKTARITCGGYNIGSKFVHWYRQRPGQAPVLVVYDDRDRPSGIPERFSGSNSGNTATLTISRVEAGDEADYYCQVWDSSNDLLIFGGGTKLTVL

SEQ ID NO: 277 - protein, 108 aa, synthetic construct
SYELTQPPSVSVAPGKTARITCGGDNIGSKSVHWYQQKPGQAPVLVVYDDSDRPSGIPERFSGSNSGNTATLTINRVEAGDEADYYCQVWDSSNDLLIFGGGTKLTVL

SEQ ID NO: 278 - protein, 108 aa, synthetic construct
SYVLTQPPSVSVAPGKTARITCGGNNTGSKSVHWYQQKPGQAPVLVVYDDSDRPSGIPERFSGSNSGNTATLTINRVEAGDEADYYCQVWDSSNDLLIFGGGTKLTVL

SEQ ID NO: 279 - protein, 108 aa, synthetic construct
SYVLTQPPSVSVAPGQTARITCGGSDIGDEIVHWYQQKPGQAPVLVVYDDSDRPSGIPERFSGSNSGNTATLTISRAQAGDEADYYCQVWDSSSDHLIFGGGTKLTVL

SEQ ID NO: 280 - protein, 108 aa, synthetic construct
SYVLTQPPSVSVAPGKTARITCGGNNIGSKSVHWYQQKPGQAPVLVVYDDSDRPLGIPERFSGSNSGNTATLTINRVEAGDEADYYCQVWDSSNDLLIFGGGTKLTVL

SEQ ID NO: 281 - protein, 108 aa, synthetic construct
SYVLTQPLSVSVALGQTARITCGGSNIGDIRVHWYQQKPGQAPVLVVYDDSDRPSGIPERFSGSNSGNTATLTINRVEAGDEADYYCQVWDSSNDLLIFGGGTKLTVL

SEQ ID NO: 282 - protein, 108 aa, synthetic construct
SYVLTQPPSVSVAPGKTARITCGGNNIGSKGVHWYQQKPGQAPVLVVYDDSDRPSGIPERFSGSNSGNTATLTINRVEAGDEADYYCQVWDSSNDLLIFGGGTKLTVL

SEQ ID NO: 283 - protein, 108 aa, synthetic construct
SYVLTQPPSVSVAPGQTARVSCGGNNIGSKSVNWYRQKPGQAPVLVIYDDSDRPSGIPERFSGSNSGNTATLTISRVEAGDEADYYCQVWDSSNDLLIFGGGTKLTVL

SEQ ID NO: 284 - protein, 108 aa, synthetic construct
SYVLTQPPSVSVAPGKTARITCGGNNIGSKNVHWYQQKPGQAPVLVVYDDSDRPSGIPERFSGSNSGNTATLTINRVEAGDEADYYCQVWDSSNDLLIFGGGTKLTVL

SEQ ID NO: 285 - protein, 108 aa, synthetic construct
SYVLTQPPSVSVAPGQTARITCGGNNIGIKSVHWYQQKPGQAPVLVVYDDSDRPSGIPERFSGSNSGNTATLTINRVEAGDEADYYCQVWDSSNDLLIFGGGTKLTVL

SEQ ID NO: 286 - protein, 108 aa, synthetic construct
SYVLTQPPSVSVAPGKTARITCGGNNIGSKSVHWYQQKPGQAPVLVVYDDSDRPSGIPERFSGSNSGNTATLTINRVEAGDEADYYCQVWDSSNDLLIFGGGTKLTVL

SEQ ID NO: 287 - protein, 108 aa, synthetic construct
SYELTQPPSVAVAPGQTARITCGGNNIGRKAVHWYQQKPGQAPVLVVYDDSDRPSGIPERFSGSNSGNTATLTINRVEAGDEADYYCQVWDSSNDLLIFGGGTKLTVL

SEQ ID NO: 288 - protein, 108 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSIDHLIFGGGTKVTVL

SEQ ID NO: 289 - protein, 108 aa, synthetic construct
SYVLTQPPSVSVAPGQTARITCGGNNIGIKSVHWYQQKPGQAPVLVVYDDRDRPSGIPERFSGSNSGNTATLTINRVEAGDEADYYCQVWDSSNDLLIFGGGTKLTVL

SEQ ID NO: 290 - protein, 108 aa, synthetic construct
SYELTQPPSVSVAPGQTARITCGGNNIGSKSVHWYQQKPGQAPVLVVYDDSDRPSGIPERFSGSNSGNTATLTISRVEAGDEADYYCQVWDADSDQLIFGGGTKLTVL

SEQ ID NO: 291 - protein, 108 aa, synthetic construct
SYVLTQPPSVSVAPGKTARITCGGNNIGSKSVHWYQQKPGQAPVLVVYDDSDRPSGIPERFSGSNSGNTATLTISRVEAGDEADYYCQVWDSSSDLLIFGGGAKLTVL

SEQ ID NO: 292 - protein, 108 aa, synthetic construct
SYVLTQPPSVSVAPGKTATITCGGNNIGTKSVHWYQQKPGQAPVLVVYDDSDRPSGIPERFSGSNSGNTATLTINRVEAGDEADYYCQVWDSSNDLLIFGGGTKLTVL

SEQ ID NO: 293-314 - skipped (22 entries)

SEQ ID NO: 315 - protein, 111 aa, synthetic construct
SYVLTQPPSVSVAPGKTARITCTGTSSDVGGYNYVSWYQQKPGQAPVLVVYEVSNRPSGIPERFSGSNSGNTATLTISRVEAGDEADYYCQVWDSSNDLLIFGGGTKLTVL

SEQ ID NO: 316 - protein, 108 aa, synthetic construct
SYVLTQPPSVSVAPGKTARITCGGNNIGSKSVHWYQQKPGQAPVLVVYDDSDRPSGIPERFSGSNSGNTATLTISRVEAGDEADYYCQVWDSSNDLLIFGGGTKLTVL

SEQ ID NO: 317-318 - skipped (2 entries)

SEQ ID NO: 319 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 320 - protein, 411 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 321 - protein, 430 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDNCRRMMRTKMRMRRMRRTRRKMRRKMSPARPRTSCREACLQGWTEA

SEQ ID NO: 322 - protein, 36 aa, synthetic construct
RMRRMRRTRRKMRRKMSPARPRTSCREACLQGWTEA

SEQ ID NO: 323-328 - skipped (6 entries)

SEQ ID NO: 329 - protein, 10 aa, synthetic construct
GYTLTELSMQ

SEQ ID NO: 330-346 - skipped (17 entries)

SEQ ID NO: 347 - protein, 4 aa, unidentified
KDEL

(19) United States
(12) Patent Application Publication
(10) Pub. No.: US 2026/0306462 A1
(43) Pub. Date: Oct. 8, 2026

(54) TREATMENT OF CLONAL HEMATOPOIETIC DISORDERS USING KINASE INHIBITORS

(71) Applicant: Incyte Corporation, Wilmington, DE (US)

(72) Inventors: Ricardo Macarrón Larumbe, Malvern, PA (US); Ekaterine Asatiani, Founex (CH); Hang Thi Dang, Wilmington, DE (US); Brent Douty, Fallowfield, PA (US); Xin Li, Claymont, DE (US); Michael Liang, Wilmington, DE (US); Patrick Mayes, Wilmington, DE (US); Minh Nguyen, Claymont, DE (US); Evan Styduhar, Media, PA (US); Oleg Vechorkin, Wilmington, DE (US); Anlai Wang, Wilmington, DE (US); Xiaozhao Wang, Moorestown, NJ (US); Hai Fen Ye, Newark, DE (US); Eddy W. Yue, Landenberg, PA (US); Peng Zhao, Newark, DE (US)

(21) Appl. No.: 19/426,708

(22) Filed: Dec. 19, 2025

Related U.S. Application Data
(60) Provisional application No. 63/737,048, filed on Dec. 20, 2024.

(51) Int. Cl. A61K 31/437; A61P 7/02; C07K 16/18

(52) U.S. Cl. CPC A61K 31/437; A61P 7/02; C07K 16/18

(57) ABSTRACT

The present application provides methods for the treatment of clonal hematopoietic disorders using compounds that modulate kinase activity or antibodies that binds to human mutant calreticulin (CALR).

### SEQUENCE LISTING

The instant application contains a Sequence Listing which has been submitted electronically in XML format and is hereby incorporated by reference in its entirety. Said XML copy, created on Dec. 16, 2025, is named 20443-0860001_SL.xml and is 224,100 bytes in size.

### TECHNICAL FIELD

The present application provides methods for the treatment of clonal hematopoietic disorders using compounds that modulate kinase activity or antibodies that binds to human mutant calreticulin (CALR).

### BACKGROUND

Janus kinase (JAK) 2 plays pivotal roles in signaling by several cytokine receptors. The mutant JAK2 V617F, located at pseudokinase (JH2) domain, is the most common molecular event associated with myeloproliferative neoplasms (MPNs). Selective targeting of the JAK2 V617F mutant over the JAK2 kinase (JH1) domain may be useful for treating various pathologies, for instance clonal hematopoietic disorders, while sparing essential JAK2 functions. Inhibition of the JAK2 V617F mutant may benefit patients suffering from clonal hematopoietic disorders. This application is directed to that need and others.

### SUMMARY

The present application provides methods of treating a clonal hematopoietic disorder in a patient in need thereof, comprising administering to the patient a therapeutically effective amount of a compound which inhibits the activity of the V617F variant of JAK2 kinase, or a pharmaceutically acceptable salt thereof.

The present application further provides methods of treating clonal hematopoiesis of indeterminate potential in a patient in need thereof, comprising administering to the patient a therapeutically effective amount of a compound selected from:
methyl ((1R,3R)-3-(7-(3-fluoro-1-(methyl-d₃)-1H-pyrazol-4-yl)-3-(methyl-d₃)-8-(1-(methyl-d₃)-1H-indazol-5-yl)-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate;
methyl ((1R,3R)-3-(8-(1,1-dimethyl-1,3-dihydroisobenzofuran-5-yl)-7-(3-fluoro-1-(methyl-d₃)-1H-pyrazol-4-yl)-3-(methyl-d₃)-2-oxo-3,6-dihydroimidazo[4,5-d]pyrrolo[2,3-b]pyridin-1(2H)-yl)cyclopentyl)carbamate;
methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclopropylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1,2,2,5,5-d)carbamate;
methyl ((1R,3R)-3-(6-((1-(1-carbamoylcyclobutyl)-4-(trans-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-methoxycyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-7-methyl-2-(trifluoromethyl)-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1s,3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate; and
methyl ((1R,3R)-3-(6-((7-((1r,3R)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
   or a pharmaceutically acceptable salt of any of the aforementioned.

The present application also provides a compound which inhibits the activity of the V617F variant of JAK2 kinase, or a pharmaceutically acceptable salt thereof, for use in treating a clonal hematopoietic disorder (e.g., clonal hematopoiesis of indeterminate potential (CHIP)).

The present application further provides use of a compound which inhibits the activity of the V617F variant of JAK2 kinase, or a pharmaceutically acceptable salt thereof, for preparation of a medicament for use in treatment of a clonal hematopoietic disorder (e.g., clonal hematopoiesis of indeterminate potential (CHIP)).

The present application further provides a method of treating a clonal hematopoietic disorder in a patient in need thereof, comprising administering to the patient a therapeutically effective amount of an antibody that binds to human mutant calreticulin (CALR).

The present application also provides an antibody that binds to human mutant calreticulin (CALR) for use in treating a clonal hematopoietic disorder (e.g., clonal hematopoiesis of indeterminate potential (CHIP)).

The present application further provides use of an antibody that binds to human mutant calreticulin (CALR) for preparation of a medicament for use in treatment of a clonal hematopoietic disorder (e.g., clonal hematopoiesis of indeterminate potential (CHIP)).

### DESCRIPTION OF DRAWINGS

FIG. 1 shows the percentage of human neutrophils that were positive for the H3cit marker indicating presence of neutrophil extracellular traps (NETs).

FIG. 2 shows that the JAK2V617F selective inhibitor, methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate, (Compound 1), prevented NETosis levels in mice when dosed prophylactically.

FIG. 3 shows that Compound 1 reduced NETosis levels in mice when dosed after NETosis had been established in the mice.

FIGS. 4A-4E show that Compound 1 suppressed levels of inflammatory cytokines linked to neutrophil activation.

### DETAILED DESCRIPTION

The present application provides, inter alia, a method of treating clonal hematopoietic disorders in a patient in need thereof, comprising administering a therapeutically effective amount of compound which inhibits the activity of the V617F variant of JAK2 kinase, or a pharmaceutically acceptable salt thereof, or an antibody that binds to human mutant calreticulin (CALR).

The compounds described herein can inhibit the activity of the V617F variant of the protein-tyrosine kinase JAK2 (i.e., “V617F” or “JAK2V617F”). Compounds which inhibit V617F are useful in providing a means of preventing the growth or inducing apoptosis in tumors, particularly by inhibiting angiogenesis. It is therefore anticipated that the compounds of the disclosure (i.e., small molecule inhibitors of JAK2V617F and antibodies that bind to human mutant calreticulin (CALR) as described herein) are useful in treating or preventing proliferative disorders such as cancers. In particular tumors with activating mutants of receptor tyrosine kinases or upregulation of receptor tyrosine kinases may be particularly sensitive to the inhibitors.

As used herein, the term “compound” (e.g., “compounds of the present invention,” “compounds of the disclosure,” and the like) is understood to encompass small molecule inhibitors of JAK2V617F and antibodies that bind to human mutant calreticulin (CALR) as described herein.

The methods described herein can be for the treatment of clonal hematopoietic disorders, and related disorders. As used herein, “clonal hematopoiesis” refers to the presence of clonal populations of hematopoietic stem cells (HSC). Hematopoiesis is generally a polyclonal process with HSCs of equipotential, giving rise to erythroid, lymphoid, myeloid, or megakaryocytic cells. Mutations may occur in genes that confer selective fitness advantage with aging HSCs less adept to correct for these errors, giving rise to clonally expanded populations of stem cells.

The presence of mutations can lead to clonal hematopoiesis of indeterminate potential (CHIP), which may induce an altered inflammatory state that is associated with an increased risk of cardiovascular conditions, coronary artery disease, ischemic stroke, atherosclerosis, poorer outcomes in aortic stenosis and heart failure, and enhanced thrombogenesis.

Example diseases and disorders associated with clonal hematopoiesis include, but are not limited to, autoimmune diseases, atherosclerosis, cancer, Philadelphia-negative myeloproliferative neoplasm (MPN), polycythemia vera (PV), essential thrombocythemia (ET), eye disorders, chronic obstructive pulmonary disease (COPD), osteoporosis, chronic liver disease, acute kidney injury, thoracic aortic aneurysms, NETosis, NETosis-related conditions, thrombosis, cardiac diseases and disorders, myocardial infarction (MI), ulcerative colitis, inflammatory bowel disease, pericarditis, myocarditis, thromboembolis, myelodysplastic syndrome (MDS), myeloproliferative neoplasms (MPNs), chronic infections, mycobacterial infection, sepsis, pneumonia, HIV, non-Hodgkin's lymphoma, glioma, and acute myeloid leukemia.

In some embodiments, the methods provided herein comprises treating a disease or disorder associated with clonal hematopoiesis selected from autoimmune diseases, atherosclerosis, cancer, Philadelphia-negative myeloproliferative neoplasm (MPN), polycythemia vera (PV), essential thrombocythemia (ET), eye disorders, chronic obstructive pulmonary disease (COPD), osteoporosis, chronic liver disease, acute kidney injury, thoracic aortic aneurysms, NETosis, NETosis-related conditions, thrombosis, venous thromboembolism, cardiac diseases and disorders, myocardial infarction (MI), ulcerative colitis, inflammatory bowel disease, pericarditis, myocarditis, thromboembolis, myelodysplastic syndrome (MDS), myeloproliferative neoplasms (MPNs), chronic infections, mycobacterial infection, sepsis, pneumonia, HIV, non-Hodgkin's lymphoma, glioma, and acute myeloid leukemia.

As used herein, “clonal hematopoiesis of indeterminate potential (CHIP)” refers to refers to CH specifically harboring a somatic mutation in a myeloid neoplasm driver gene with a variant allele frequency (VAF)≥2% in a patient lacking a hematologic neoplasm or unexplained cytopenia. In some embodiments, the method provided herein comprises treating a disease or disorder associated with clonal hematopoiesis of indeterminate potential (CHIP).

In some embodiments, the clonal hematopoiesis of indeterminate potential is selected from CALR CHIP, DNMT3A CHIP, TET2 CHIP, ASXL1 CHIP, SRSR2 CHIP, ZRSR2 CHIP, RUNX1 CHIP, IDH2 CHIP, SF3B1 CHIP, JAK2 CHIP, JAK2 V617F CHIP, and non-JAK2 CHIP, or any combination thereof.

In some embodiments, the clonal hematopoiesis of indeterminate potential is CALR CHIP.

In some embodiments, the clonal hematopoiesis of indeterminate potential is JAK2 CHIP.

In some embodiments, the clonal hematopoiesis of indeterminate potential is JAK2 V617F CHIP.

In some embodiments, the clonal hematopoiesis of indeterminate potential (CHIP) is clonal cytopenia of undetermined significance (CCUS).

As used herein, “clonal cytopenia of undetermined significance (CCUS)” refers to CHIP detected in the presence of one or more persistent unexplained cytopenias, while diagnostic criteria for any defined myeloid neoplasm are not met.

In some embodiments, the method provided herein comprises treating a disease or disorder associated with clonal cytopenia of undetermined significance (CCUS).

In some embodiments, the present disclosure provides a method of reducing the risk of recurrence of a disease or disorder provided herein (e.g., a disease or disorder associated with clonal hematopoiesis of indeterminate potential (CHIP)) in a patient who has had at least one indication event (e.g., a cardiovascular event). In some embodiments, the method further comprises identifying the patient who has had at least one indication event; determining the presence of the mutant JAK2 V617F in the patient; selecting a patient having the mutant JAK2 V617F; and administering to the patient an effective amount of a compound provided herein, or a pharmaceutically acceptable salt thereof. In some embodiments, the patient has been identified (e.g., diagnosed) as having clonal hematopoiesis of indeterminate potential (CHIP).

The methods described herein include methods for the treatment of clonal hematopoiesis of indeterminate potential (CHIP) patients (e.g., patients diagnosed as having CHIP) who have one or more of the following diseases or disorders, or who are at higher risk of developing one or more of the following diseases or disorders: autoimmune diseases (e.g., antineutrophil cytoplasmic antibody (ANCA)-associated vasculitis (AAV), systemic lupus erythematosus (SLE), rheumatoid arthritis (RA), psoriasis, antiphospholipid syndrome (APS), multiple sclerosis (MS), dermatomyositis (DM), polymyositis (PM), IgG4-related autoimmune pancreatitis (AIP), drug-induced autoimmune diseases (see e.g., He et al, Chin. Med. J. (Engl). 2018, Jul. 5; 131(13):1513-1519; Lee et al., Autoimmun. Rev. 2017 November; 16(11):1160-1173)); atherosclerosis; cancer (see e.g., Olsson and Cedarvall, Front Immunol. 2016; 7: 373; Cedarvall and Olsson, Oncoscience, 2015; 2(11): 900-901; Genovse et al, N. Engl. J. Med. 2014; 371:2477-2487; Cordua et al, Blood, 2019; 134(5):469-479); Philadelphia-negative myeloproliferative neoplasm (MPN), e.g., polycythemia vera (PV) or essential thrombocythemia (ET); eye disorders, such as drusen and age-related macular degeneration (see e.g., EClinicalMedicine, 2022; 43: 101248); chronic obstructive pulmonary disease (COPD) (see e.g., Miller et al, Blood, 2022; 139:357-6839); osteoporosis (see e.g., Kim et al. J. Exp. Med., 2021, 218:e2021187); chronic liver disease (see e.g., Wong et al., Nature, 2023, 616(7958):747-754); acute kidney injury (see e.g., Vlasschaert et al., Nature Med. 2024; 30: 810-817); and thoracic aortic aneurysms (see e.g., Nakao et al., J. Am. Coll. Cardiol. 2023; 81(21): 2128-2130).

In some embodiments, the methods provided herein can be used, e.g., for reducing NETosis, or reducing the risk of NETosis-related conditions, e.g., treating or reducing the risk of occurrence or reoccurrence of a cardiovascular event (e.g., thrombosis or myocardial infarction (MI)). Generally, the methods include administering a therapeutically effective amount of an inhibitor of the present disclosure, to a patient who is in need of, or who has been determined to be in need of, such treatment. In some embodiments, the methods described herein further comprise determining the patient's Jak2 Genotype, i.e., determining whether the patient has one or more JAK2V617F/I alleles, and selecting and treating patients who do have one or more JAK2V617F/I alleles. In some embodiments, the patient does not have one or more JAK2V617F/I alleles.

In some embodiments, the method comprises treating a JAK2 V617F CHIP-induced disease in the patient.

In some embodiments, the JAK2 V617F CHIP-induced disease is selected from hematologic cancer, myeloproliferative neoplasm, myeloid cancer, coronary heart disease, stroke, ischemic heart disease, venous thromboembolism, pulmonary embolism, drusen, age-related macular degeneration, chronic obstructive pulmonary disease (COPD), osteoporosis, chronic liver disease, acute kidney injury, thoracic aortic aneurysm, leukocytosis, neutropenia, hyperplasia, arteriosclerosis, phlebosclerosis caused by activated neutrophils, aortic aneurysm, hypertension, pulmonary hypertension, myeloid leukemia, lymphocytic leukemia, myelodysplastic syndrome, aplastic anemia, paroxysmal nocturnal hemoglobinuria, malignant lymphoma, and multiple myeloma.

In some embodiments, the CHIP-induced disease is selected from acute kidney injury, chronic liver disease, thoracic aortic aneurysm, coronary heart disease, and hematological malignancy.

In some embodiments, the CHIP-induced disease is acute kidney injury.

In some embodiments, the CHIP-induced disease is chronic liver disease.

In some embodiments, the CHIP-induced disease is thoracic aortic aneurysm.

In some embodiments, the CHIP-induced disease is coronary heart disease.

In some embodiments, the CHIP-induced disease is a hematological malignancy.

In some embodiments, the method comprises treating JAK2 V617F CHIP-induced thrombosis in the patient.

In some embodiments, the JAK2 V617F CHIP-induced thrombosis comprises arterial thrombosis.

In some embodiments, the JAK2 V617F CHIP-induced thrombosis comprises venous thrombosis.

In some embodiments, the method comprises treating JAK2 V617F CHIP-induced atherosclerosis in the patient.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a V617F variant with a compound described herein includes the administration of a compound described herein to an individual or patient, such as a human, having a V617F variant, as well as, for example, introducing a compound described herein into a sample containing a cellular or purified preparation containing the V617F variant.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention (i.e., small molecule inhibitors of JAK2V617F and antibodies that bind to human mutant calreticulin (CALR) as described herein) are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### JAK2 V617F Inhibitors

In some embodiments, the methods provided comprise administering to the patient a compound of Formula I:

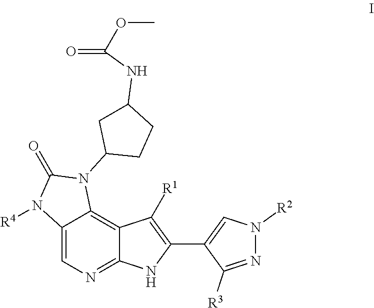

or a pharmaceutically acceptable salt thereof, wherein:
R¹ is selected from phenyl, indazolyl, and dihydroisobenzofuranyl, each of which is optionally substituted by 1, 2, or 3 substituents independently selected from halo, C₁₋₆ alkyl, C₁₋₆ alkoxy, C₁₋₆ haloalkyl, and C₁₋₆ hydroxyalkyl;
R² is C₁₋₆ alkyl or C₁₋₆ haloalkyl;
R³ is selected from halo, C₁₋₆ alkyl, and C₁₋₆ alkoxy; and
R⁴ is C₁₋₆ alkyl.

In some embodiments, the compound is a compound of Formula I, wherein:
R¹ is selected from phenyl, indazolyl, and dihydroisobenzofuranyl, each of which is optionally substituted by 1 or 2 substituents independently selected from halo, C₁₋₃ alkyl, C₁₋₃ alkoxy, C₁₋₃ haloalkyl, and C₁₋₃ hydroxyalkyl;
R² is C₁₋₃ alkyl or C₁₋₃ haloalkyl;
R³ is selected from halo, C₁₋₃ alkyl, and C₁₋₃ alkoxy; and
R⁴ is C₁₋₃ alkyl.

In some embodiments, the compound is a compound of Formula I, wherein:
R¹ is selected from phenyl, indazolyl, and dihydroisobenzofuranyl, each of which is optionally substituted by 1 or 2 substituents independently selected from fluoro, methyl, isopropyl, trideuteromethyl, difluoroethyl, methoxy, trideuteromethoxy, and hydroxyisopropyl;
R² is selected from methyl, ethyl, trideuteromethyl, difluoroethyl, pentadeuteroethyl, and heptadeuteroisopropyl;
R³ is methoxy or fluoro; and
R⁴ is methyl or trideuteromethyl.

In some embodiments, the compound is a compound of Formula I, wherein:
R¹ is selected from phenyl and indazolyl, each of which is optionally substituted by 1 or 2 substituents independently selected from halo, C₁₋₃ alkyl, and C₁₋₃ alkoxy;
R² is C₁₋₃ alkyl;
R³ is selected from halo, C₁₋₃ alkyl, and C₁₋₃ alkoxy; and
R⁴ is C₁₋₃ alkyl.

In some embodiments, the compound is a compound of Formula I, wherein:
R¹ is selected from phenyl and indazolyl, each of which is optionally substituted by 1 or 2 substituents independently selected from fluoro, trideuteromethyl, and methoxy;
R² is methyl or trideuteromethyl;
R³ is methoxy or fluoro; and
R⁴ is methyl or trideuteromethyl.

In some embodiments, the compound of Formula I is a compound of Formula Ia:

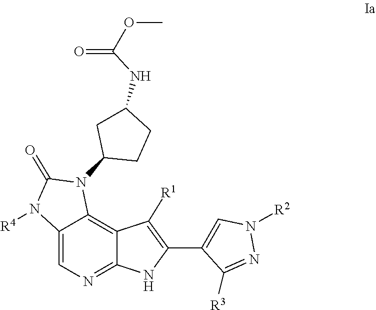

or a pharmaceutically acceptable salt thereof.

In some embodiments, the compound of Formula I is a compound of Formula II:

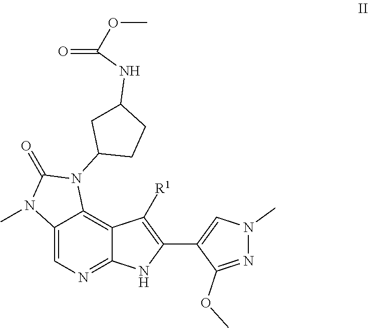

or a pharmaceutically acceptable salt thereof.

In some embodiments, the compound of Formula I is a compound of Formula IIa:

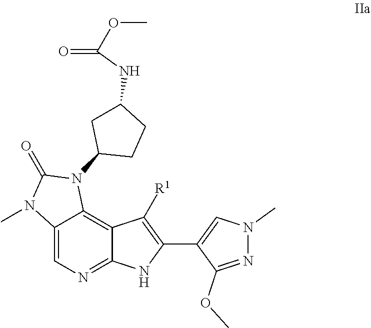

or a pharmaceutically acceptable salt thereof.

In some embodiments, the compound of Formula I is a compound of Formula III:

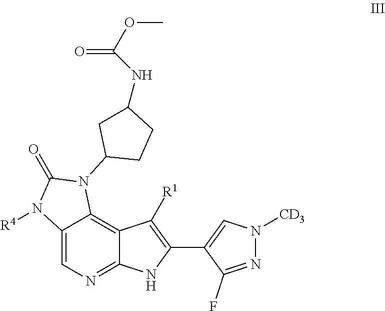

or a pharmaceutically acceptable salt thereof.

In some embodiments, the compound of Formula I is a compound of Formula IIIa:

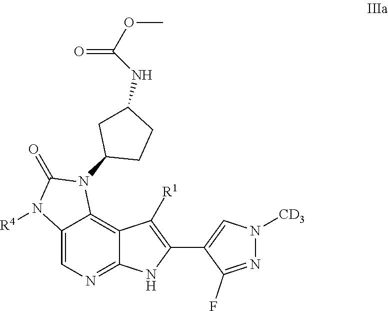

or a pharmaceutically acceptable salt thereof.

In some embodiments, the compound of Formula I is a compound of Formula IV:

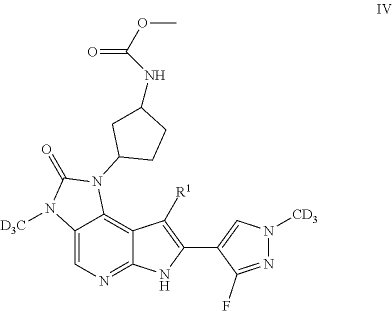

or a pharmaceutically acceptable salt thereof.

In some embodiments, the compound of Formula I is a compound of Formula IVa:

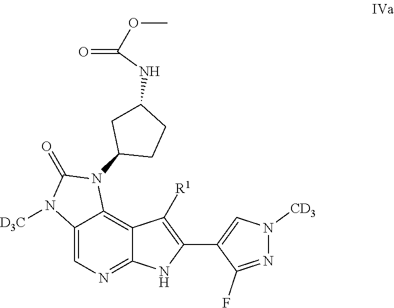

or a pharmaceutically acceptable salt thereof.

In some embodiments, the compound of Formula I is a compound of Formula V:

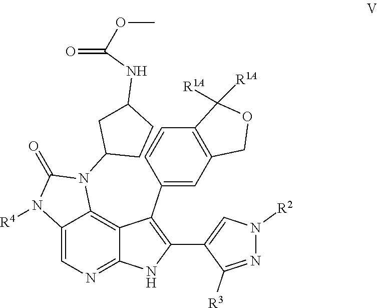

or a pharmaceutically acceptable salt thereof, wherein each R^{1A} is independently selected from halo, C₁₋₆ alkyl, C₁₋₆ alkoxy, C₁₋₆ haloalkyl, and C₁₋₆ hydroxyalkyl.

In some embodiments, the compound of Formula I is a compound of Formula Va:

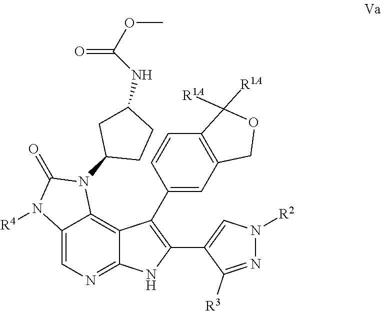

or a pharmaceutically acceptable salt thereof, wherein each R^{1A} is independently selected from halo, C₁₋₆ alkyl, C₁₋₆ alkoxy, C₁₋₆ haloalkyl, and C₁₋₆ hydroxyalkyl.

In some embodiments, the compound of Formula I is a compound of Formula Vb:

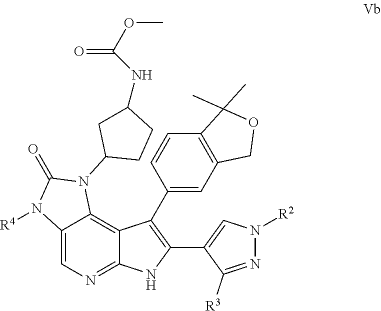

or a pharmaceutically acceptable salt thereof.

In some embodiments, the compound of Formula I is a compound of Formula Vc:

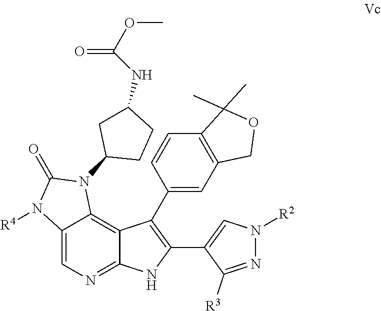

or a pharmaceutically acceptable salt thereof.

In some embodiments, the compound of Formula I is selected from:
methyl ((1R,3R)-3-(7-(3-fluoro-1-(methyl-d₃)-1H-pyrazol-4-yl)-3-(methyl-d₃)-8-(1-(methyl-d₃)-1H-indazol-5-yl)-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate; and
methyl ((1R,3R)-3-(8-(1,1-dimethyl-1,3-dihydroisobenzofuran-5-yl)-7-(3-fluoro-1-(methyl-d₃)-1H-pyrazol-4-yl)-3-(methyl-d₃)-2-oxo-3,6-dihydroimidazo[4,5-d]pyrrolo[2,3-b]pyridin-1(2H)-yl)cyclopentyl)carbamate;
   or a pharmaceutically acceptable salt of any of the aforementioned.

In some embodiments, the compound of Formula I is methyl ((1R,3R)-3-(7-(3-fluoro-1-(methyl-d₃)-1H-pyrazol-4-yl)-3-(methyl-d₃)-8-(1-(methyl-d₃)-1H-indazol-5-yl)-2-oxo-3,6-dihydroimidazo[4,5-d]pyrrolo[2,3-b]pyridin-1(2H)-yl)cyclopentyl)carbamate, or a pharmaceutically acceptable salt thereof.

In some embodiments, the compound of Formula I is methyl ((1R,3R)-3-(7-(3-fluoro-1-(methyl-d₃)-1H-pyrazol-4-yl)-3-(methyl-d₃)-8-(1-(methyl-d₃)-1H-indazol-5-yl)-2-oxo-3,6-dihydroimidazo[4,5-d]pyrrolo[2,3-b]pyridin-1(2H)-yl)cyclopentyl)carbamate.

In some embodiments, the compound of Formula I is methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate, or a pharmaceutically acceptable salt thereof.

In some embodiments, the compound of Formula I is methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate.

In some embodiments, the compound of Formula I is methyl ((1R,3R)-3-(8-(1,1-dimethyl-1,3-dihydroisobenzofuran-5-yl)-7-(3-fluoro-1-(methyl-d₃)-1H-pyrazol-4-yl)-3-(methyl-d₃)-2-oxo-3,6-dihydroimidazo[4,5-d]pyrrolo[2,3-b]pyridin-1(2H)-yl)cyclopentyl)carbamate, or a pharmaceutically acceptable salt thereof.

In some embodiments, the compound of Formula I is methyl ((1R,3R)-3-(8-(1,1-dimethyl-1,3-dihydroisobenzofuran-5-yl)-7-(3-fluoro-1-(methyl-d₃)-1H-pyrazol-4-yl)-3-(methyl-d₃)-2-oxo-3,6-dihydroimidazo[4,5-d]pyrrolo[2,3-b]pyridin-1(2H)-yl)cyclopentyl)carbamate.

In some embodiments, the method comprises administering to the patient a compound of Formula A:

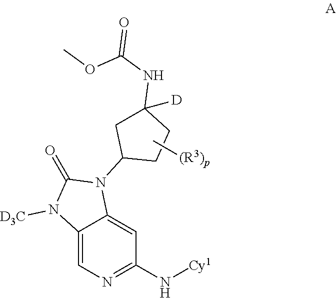

or a pharmaceutically acceptable salt thereof, wherein:
Cy¹ is selected from

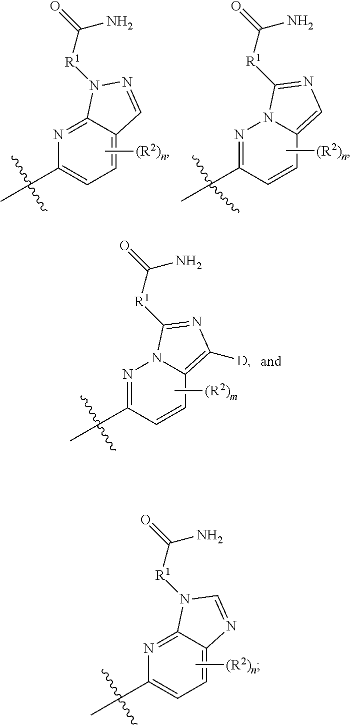

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 substituents independently selected from halo, oxo, C₁₋₃ alkyl, C₁₋₃ haloalkyl, C₁₋₃ alkoxy, C₁₋₃ haloalkoxy, CN, OH, C(O)OH, and NH₂;
each R² is independently selected from halo, oxo, C₁₋₆ alkyl, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, CN, OR^{a21}, C(O)NR^{c21}R^{d21}, OC(O)NR^{c21}R^{d21}, NR^{c21}R^{d21}, NR^{c21}C(O)R^{b21}, NR^{c21}C(O)OR^{a21}, NR^{a21}C(O)NR^{c21}R^{d21}, NR²¹S(O)₂R^{b21}, NR^{c21}S(O)(═NR^{e21})R^{b21}, NR^{c21}S(O)₂NR^{c21}R^{d21}, S(O)₂R^{b21}, S(O)₂NR^{c21}R^{d21}, OS(O)(═NR^{e21})R^{b21}, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, or 4 independently selected R^{2A} substituents;
or, any R^{c21}, and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
each R^{b21} is independently selected from H, C₁₋₆ alkyl, C₁₋₆ haloalkyl, C₂₋₆ alkenyl, C₂-6 alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, or 4 independently selected R^{2A} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{2A} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, or 4 independently selected R^{M} substituents;
each R³ is independently selected from halo, oxo, C₁₋₃ alkyl, C₁₋₃ haloalkyl, C₁₋₃ alkoxy, C₁₋₃ haloalkoxy, CN, OH, C(O)OH, and NH₂;
m is 0, 1, or 2;
n is 0, 1, 2, or 3;
p is 0, 1, or 2; and
each R^{M} is independently selected from OH, halo, oxo, CN, C(O)OH, NH₂, NO₂, SF₅, C₁₋₆ alkyl, C₁₋₆ alkoxy, C₁₋₆haloalkoxy, C₁₋₆haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments, the compound is a compound of Formula A, or a pharmaceutically acceptable salt thereof, wherein Cy¹ is

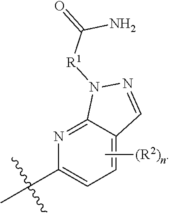

In some embodiments, the compound is a compound of Formula A, or a pharmaceutically acceptable salt thereof, wherein Cy¹ is

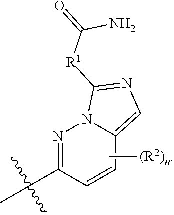

In some embodiments, the compound is a compound of Formula A, or a pharmaceutically acceptable salt thereof, wherein Cy¹ is

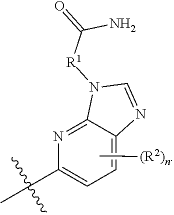

In some embodiments, the compound is a compound of Formula A, or a pharmaceutically acceptable salt thereof, wherein Cy¹ is

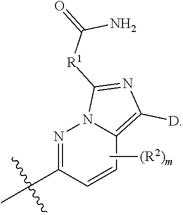

In some embodiments, the compound is a compound of Formula A, or a pharmaceutically acceptable salt thereof, wherein:
Cy¹ is selected from

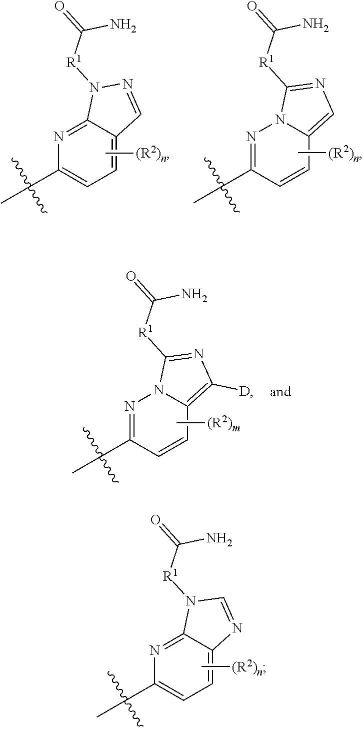

R¹ is selected from —C₃₋₅ cycloalkyl- and —(C₃₋₅ cycloalkyl)-methyl-, wherein the —C₃₋₅ cycloalkyl- and —(C₃₋₅ cycloalkyl)-methyl- are each optionally substituted with 1, 2, or 3 substituents independently selected from halo, C₁₋₃ alkyl, and C₁₋₃ alkoxy;
each R² is independently selected from C₁₋₆ alkyl, C₁₋₆ haloalkyl, C₃₋₇ cycloalkyl, and 4-7 membered heterocycloalkyl, wherein each C₁₋₆ alkyl, C₃₋₇ cycloalkyl, and 4-7 membered heterocycloalkyl is optionally substituted with 1 or 2 hydroxy groups;
m is 0, 1, or 2;
n is 0, 1, 2, or 3; and
p is 0, 1, or 2.

In some embodiments, the compound is a compound of Formula A, or a pharmaceutically acceptable salt thereof, wherein:
Cy¹ is selected from

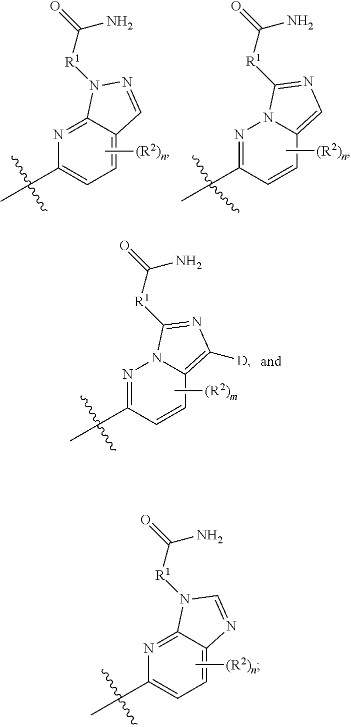

R¹ is selected from R¹ is selected from -cyclobutyl-, -cyclopropyl-, and -cyclopropylmethyl-, wherein the -cyclobutyl-, -cyclopropyl-, and -cyclopropylmethyl- are each optionally substituted with 1, 2, or 3 substituents independently selected from halo, C₁₋₃ alkyl, and C₁₋₃ alkoxy;
each R² is independently selected from methyl, ethyl, n-propyl, isopropyl, difluoromethyl, trifluoromethyl, cyclopropyl, cyclobutyl, cyclohexyl, and tetrahydropyranyl, wherein the methyl, ethyl, n-propyl, isopropyl, cyclopropyl, cyclobutyl, cyclohexyl, and tetrahydropyranyl of R² are each optionally substituted with 1 or 2 hydroxy groups;
m is 0;
n is 0, 1, or 2; and
p is 0.

In some embodiments, the compound is a compound of Formula A, or a pharmaceutically acceptable salt thereof, wherein:
Cy¹ is selected from

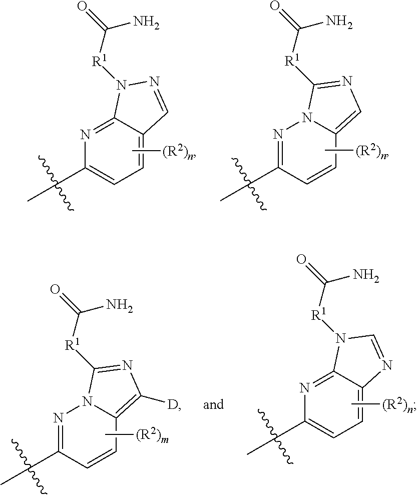

R¹ is selected from

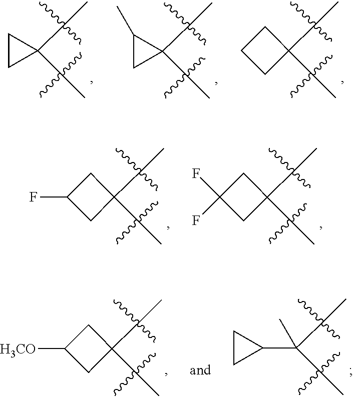

each R² is independently selected from methyl, hydroxyethyl, hydroxypropyl, hydroxyisopropyl, difluoromethyl, trifluoromethyl, cyclopropyl, cyclobutyl, hydroxycyclohexyl, and tetrahydropyranyl;
m is 0;
n is 0, 1, or 2; and
p is 0.

In some embodiments, the compound of Formula A is a compound of Formula B:

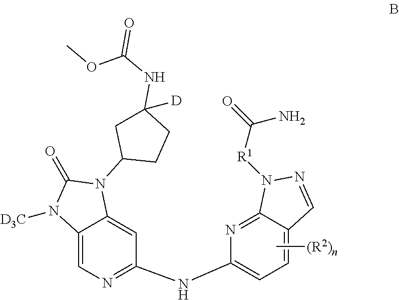

or a pharmaceutically acceptable salt thereof.

In some embodiments, the compound of Formula A is a compound of Formula C:

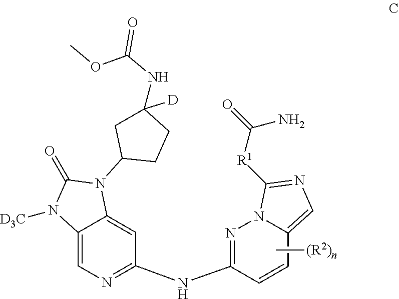

or a pharmaceutically acceptable salt thereof.

In some embodiments, the compound of Formula A is a compound of Formula D:

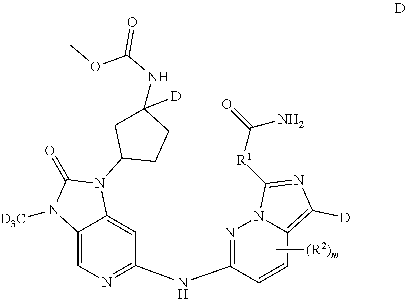

or a pharmaceutically acceptable salt thereof.

In some embodiments, the compound of Formula A is a compound of Formula E:

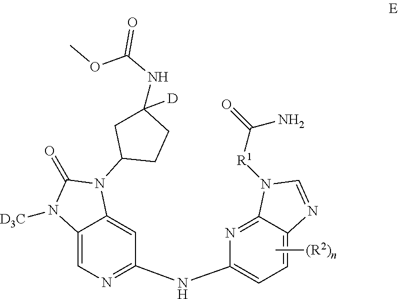

or a pharmaceutically acceptable salt thereof.

In some embodiments, the compound of Formula A is a compound of Formula F:

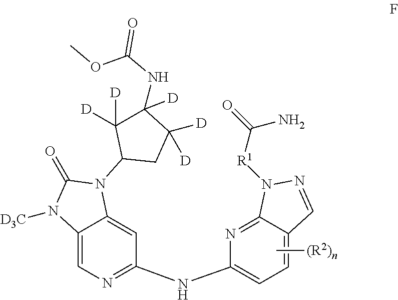

or a pharmaceutically acceptable salt thereof.

In some embodiments, the compound of Formula A is selected from:
methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclopropylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1,2,2,5,5-d₅)carbamate;
methyl ((1R,3R)-3-(6-((1-(1-carbamoylcyclobutyl)-4-(trans-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-methoxycyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-7-methyl-2-(trifluoromethyl)-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1s,3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate; and
methyl ((1R,3R)-3-(6-((7-((1r,3R)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
   or a pharmaceutically acceptable salt of any of the aforementioned.

In some embodiments, the compound of Formula A is methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate, or a pharmaceutically acceptable salt thereof.

In some embodiments, the compound of Formula A is methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate.

In some embodiments, the compound of Formula A is methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate, or a pharmaceutically acceptable salt thereof.

In some embodiments, the compound of Formula A is methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate.

In some embodiments, the compound of Formula A is methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate, or a pharmaceutically acceptable salt thereof.

In some embodiments, the compound of Formula A is methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate.

In some embodiments, the method provided herein comprises treating clonal hematopoiesis of indeterminate potential in a patient in need thereof, comprising administering to the patient a therapeutically effective amount of a compound selected from:
methyl ((1R,3R)-3-(7-(3-fluoro-1-(methyl-d₃)-1H-pyrazol-4-yl)-3-(methyl-d₃)-8-(1-(methyl-d₃)-1H-indazol-5-yl)-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate;
methyl ((1R,3R)-3-(8-(1,1-dimethyl-1,3-dihydroisobenzofuran-5-yl)-7-(3-fluoro-1-(methyl-d₃)-1H-pyrazol-4-yl)-3-(methyl-d₃)-2-oxo-3,6-dihydroimidazo[4,5-d]pyrrolo[2,3-b]pyridin-1(2H)-yl)cyclopentyl)carbamate;
methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclopropylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1,2,2,5,5-d₅)carbamate;
methyl ((1R,3R)-3-(6-((1-(1-carbamoylcyclobutyl)-4-(trans-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-methoxycyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-7-methyl-2-(trifluoromethyl)-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1s,3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate; and
methyl ((1R,3R)-3-(6-((7-((1r,3R)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
   or a pharmaceutically acceptable salt of any of the aforementioned.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. The term “Cₙ₋ₘ alkyl” is understood to include deuterated analogs of alkyl groups as defined herein, including but not limited to, groups such as trideuteromethyl (CD₃), pentadeuteroethyl (CD₂CD₃), and the like.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms. The term “Cₙ₋ₘ alkenyl” is understood to include deuterated analogs of alkenyl groups as defined herein, including but not limited to, groups such as trideuteroethenyl (-CD=CD₂), tetradeuteropropenyl, (-CD=CD-CD₂), and the like.

As used herein, “Cₙ₋ₘ alkynyl” refers to an alkyl group having one or more triple carbon-carbon bonds and having n to m carbons. Example alkynyl groups include, but are not limited to, ethynyl, propynyl (e.g., propyn-1-yl, propyn-2-yl, prop-2-yn-1-yl), and the like. In some embodiments, the alkynyl moiety contains 2 to 6, 2 to 4, or 2 to 3 carbon atoms. The term “Cₙ₋ₘ alkynyl” is understood to include deuterated analogs of alkynyl groups as defined herein, including but not limited to, groups such as deuteroethynyl (—C≡CD), trideuteropropyn-1-yl, (—C≡CCD3), and the like.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms. The term “Cₙ₋ₘ alkoxy” is understood to include deuterated analogs of the alkyl moiety of the alkoxy group as defined herein, including but not limited to, groups such as trideuteromethoxy (-OCD₃), pentadeuteroethoxy (-OCD₂CD₃), and the like.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl. The term “aryl” is understood to include deuterated analogs of the aryl groups as defined herein, including but not limited to, groups such as pentadeuterophenyl (i.e., perdeuterophenyl, phenyl-d₅), perdeuteronaphthyl, and the like.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms. The term “Cₙ₋ₘ haloalkoxy” is understood to include deuterated analogs of the haloalkyl moiety of the haloalkoxy group as defined herein, including but not limited to, groups such as deuterodifluoromethoxy (-OCDF₂), dideuterofluoromethoxy (-OCD₂F), and the like.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like. The term “Cₙ₋ₘ haloalkyl” is understood to include deuterated analogs of the haloalkyl group as defined herein, including but not limited to, groups such as deuterodifluoromethyl (-CDF₂), dideuterofluoromethyl (-CD₂F), and the like.

As used herein, the term “carbonyl”, employed alone or in combination with other terms, refers to a —C(O)— group.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carboxy” refers to a group of formula —C(O)OH.

As used herein, the term “di(Cₙ₋ₘ alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, azaspiro[2.4]heptanyl, and the like. In some embodiments, cycloalkyl is cyclopropyl, cyclobutyl, cyclopentyl, or cyclohexyl. The term “cycloalkyl” is understood to include deuterated analogs of the cycloalkyl groups as defined herein, including but not limited to, groups such as perdeuterocyclopropyl, perdeuterocyclobutyl, perdeuterocyclopentyl, perdeuterocyclohexyl, and the like.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, pyrazolo[1,5-a]pyrimidinyl, indazolyl, imidazo[1,2-b]pyridazinyl, pyrazolo[1,5-a]pyrimidinyl, and the like. The term “heteroaryl” is understood to include deuterated analogs of the heteroaryl groups as defined herein, including but not limited to, groups such as perdeuteropyridinyl, perdeuteropyrazinyl, perdeuteropyrimidinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. The term “heterocycloalkyl” is understood to include deuterated analogs of the heterocycloalkyl groups as defined herein, including but not limited to, groups such as perdeuteroazetidinyl, perdeuteropyrrolidinyl, perdeuteropiperidinyl, and the like.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyranyl, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazepine, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 5,6-dihydro-4H-pyrrolo[1,2-b]pyrazolyl, 3-oxopiperazinyl, oxo-pyrrolidinyl, oxo-pyridinyl, 2,3-dihydrobenzo[b][1,4]dioxinyl, 3,4-dihydro-2H-benzo[b][1,4]oxazinyl, octahydro-2H-pyrido[1,2-a]pyrazinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like. The terms “alkyl linking group” and “alkylene linking group” are understood to include deuterated analogs of the alkylene groups as defined herein.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula Ia, etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As will be appreciated by those skilled in the art, the compounds provided herein, including salts and stereoisomers thereof, can be prepared using known organic synthesis techniques and can be synthesized according to any of numerous possible synthetic routes.

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Mutant CALR and Anti-mutCALR Antibodies

CALR is a highly conserved chaperone protein that resides primarily in the endoplasmic reticulum and is involved in a variety of cellular processes including protein folding, calcium homeostasis, cell adhesion, and integrin signaling. Mutations in the CALR gene have been identified in patients with myeloproliferative neoplasms. The two most frequent CALR mutations are a 52 base pair (bp) deletion and a 5 bp insertion, which are referred to as Type 1 and Type 2 mutations, respectively. Type 1 and Type 2 mutations cause a +1 frameshift within exon 9 that generates a novel, positively-charged C-terminal amino acid sequence that lacks the KDEL domain (SEQ ID NO:347) of the wild-type (WT) protein, thereby enabling the mutCALR to escape the ER and activate the thrombopoietin receptor (MPL) and induce constitutive activation of Janus kinase 2 (JAK2) signaling. The amino acid sequences of human WT CALR, Type 1 and Type 2 mutCALR, and the novel C-terminal sequence are shown below. The frameshift amino acid residues in Type 1 and Type 2 mutCALR are shown in bold and the novel C-terminal sequence is marked by underlining.

Human WT CALR (GenBank Accession No. NP_004334.1)
(SEQ ID NO: 319)
MLLSVPLLLGLLGLAVAEPAVYFKEQFLDGDGWTSRWIESKHKSDFGKF
VLSSGKFYGDEEKDKGLQTSQDARFYALSASFEPFSNKGQTLVVQFTVK
HEQNIDCGGGYVKLFPNSLDQTDMHGDSEYNIMFGPDICGPGTKKVHVI
FNYKGKNVLINKDIRCKDDEFTHLYTLIVRPDNTYEVKIDNSQVESGSL
EDDWDFLPPKKIKDPDASKPEDWDERAKIDDPTDSKPEDWDKPEHIPDP
DAKKPEDWDEEMDGEWEPPVIQNPEYKGEWKPRQIDNPDYKGTWIHPEI
DNPEYSPDPSIYAYDNFGVLGLDLWQVKSGTIFDNFLITNDEAYAEEFG
NETWGVTKAAEKQMKDKQDEEQRLKEEEEDKKRKEEEEAEDKEDDEDKD
EDEEDEEDKEEDEEEDVPGQAKDEL
Human Type 1 mutCALR
(SEQ ID NO: 320)
MLLSVPLLLGLLGLAVAEPAVYFKEQFLDGDGWTSRWIESKHKSDFGKF
VLSSGKFYGDEEKDKGLQTSQDARFYALSASFEPFSNKGQTLVVQFTVK
HEQNIDCGGGYVKLFPNSLDQTDMHGDSEYNIMFGPDICGPGTKKVHVI
FNYKGKNVLINKDIRCKDDEFTHLYTLIVRPDNTYEVKIDNSQVESGSL
EDDWDFLPPKKIKDPDASKPEDWDERAKIDDPTDSKPEDWDKPEHIPDP
DAKKPEDWDEEMDGEWEPPVIQNPEYKGEWKPRQIDNPDYKGTWIHPEI
DNPEYSPDPSIYAYDNFGVLGLDLWQVKSGTIFDNFLITNDEAYAEEFG
NETWGVTKAAEKQMKDKQDEEQRTRRMMRTKMRMRRMRRTRRKMRRKMS
PARPRTSCREACLQGWTEA
Human Type 2 mutCALR
(SEQ ID NO: 321)
MLLSVPLLLGLLGLAVAEPAVYFKEQFLDGDGWTSRWIESKHKSDFGKF
VLSSGKFYGDEEKDKGLQTSQDARFYALSASFEPFSNKGQTLVVQFTVK
HEQNIDCGGGYVKLFPNSLDQTDMHGDSEYNIMFGPDICGPGTKKVHVI
FNYKGKNVLINKDIRCKDDEFTHLYTLIVRPDNTYEVKIDNSQVESGSL
EDDWDFLPPKKIKDPDASKPEDWDERAKIDDPTDSKPEDWDKPEHIPDP
DAKKPEDWDEEMDGEWEPPVIQNPEYKGEWKPRQIDNPDYKGTWIHPEI
DNPEYSPDPSIYAYDNFGVLGLDLWQVKSGTIFDNFLITNDEAYAEEFG
NETWGVTKAAEKQMKDKQDEEQRLKEEEEDKKRKEEEEAEDNCRRMMRT
KMRMRRMRRTRRKMRRKMSPARPRTSCREACLQGWTEA
mutCALR C-terminal consensus mutant sequence
(SEQ ID NO: 322)
RMRRMRRTRRKMRRKMSPARPRTSCREACLQGWTEA

This disclosure provides anti-mutCALR antibodies that are useful in treating clonal hematopoietic disorders. Note that in the present disclosure, unless stated otherwise, amino acid positions assigned to CDRs and frameworks in a variable region of the anti-mutCALR antibodies are specified according to Kabat; see EA Kabat, Sequences of Proteins of Immunological Interest, U.S. Dept. of Health and Human Services, Public Health Service, National Institutes of Health, 1991, (OCoLC)1138727707.

In some embodiments, the anti-mutCALR antibody comprises an anti-mutCALR antibody that comprises one, two, three, four, five, and/or six CDRs of any one of the antibodies described herein. In some embodiments, an anti-mutCALR antibody comprises (i) one, two, and/or three heavy chain CDRs of any one of the clones presented in Tables 1-2, and/or (ii) one, two, and/or three light chain CDRs from any one of the clones presented in Tables 1-2.

In some embodiments, an anti-mutCALR antibody comprises (i) three heavy chain CDRs from any one of the clones presented in Tables 4-5, and (ii) three light chain CDRs from any one of the clones presented in Tables 4-5.

In some embodiments, an anti-mutCALR antibody comprises a Group 1 anti-mutCALR antibody described in US20230272055, the content of which is incorporated herein by reference in its entirety.

TABLE 1
CDR sequences.

| Clone No. | VH CDR1 | VH CDR2 | VH CDR3 | VL CDR1 | VL CDR2 | VL CDR3 |
|---|---|---|---|---|---|---|
| 1 | ELSMQ | GFDPDDGETMYAEKFQG | SPGYDFFDY | GGNYIGSKSVH | DDGDRPS | QVWDSISDHVV |
| | (SEQ ID NO: 1) | (SEQ ID NO: 7) | (SEQ ID NO: 18) | (SEQ ID NO: 26) | (SEQ ID NO: 53) | (SEQ ID NO: 69) |
| 2 | ELSMQ | GFDPDDGETMYAEKFQG | SPGYDFFDY | GGDNIGSKSVH | DDSDRPS | QVWDSSIDHLI |
| | (SEQ ID NO: 1) | (SEQ ID NO: 7) | (SEQ ID NO: 18) | (SEQ ID NO: 27) | (SEQ ID NO: 54) | (SEQ ID NO: 70) |
| 3 | ELSMQ | GFDPDDGETMYAERFQG | SPGYDFFDY | GGNNIGSKSVH | DDSDRPS | QVWDSSNDLLI |
| | (SEQ ID NO: 1) | (SEQ ID NO: 8) | (SEQ ID NO: 18) | (SEQ ID NO: 28) | (SEQ ID NO: 54) | (SEQ ID NO: 71) |
| 4 | ELSMQ | GFDPDDGETMYAEKFQG | SPGYDFFDY | GGNNIGSKSVH | DDSDRPS | QVWDSSNDLLI |
| | (SEQ ID NO: 1) | (SEQ ID NO: 7) | (SEQ ID NO: 18) | (SEQ ID NO: 28) | (SEQ ID NO: 54) | (SEQ ID NO: 71) |
| 5 | ELSMQ | GFDPDDGETMYAEKFQG | SPGYDFFDY | VGTNFGSKNVH | DDSDRPS | QVWDSSSDHLI |
| | (SEQ ID NO: 1) | (SEQ ID NO: 7) | (SEQ ID NO: 18) | (SEQ ID NO: 29) | (SEQ ID NO: 54) | (SEQ ID NO: 72) |
| 6 | ELSMQ | GFDPDDGETMYAEKFQG | SPGYDFFDY | TGTSSDVGGYNYVS | EVSNRPS | QVWDSSNDLLI |
| | (SEQ ID NO: 1) | (SEQ ID NO: 7) | (SEQ ID NO: 18) | (SEQ ID NO: 30) | (SEQ ID NO: 55) | (SEQ ID NO: 71) |
| 7 | ELSMQ | GFDPDDGETMYAEKFQG | SPGYDFFDY | VGTNFGSKNVH | DDSDRPS | QVWDSSNDLLI |
| | (SEQ ID NO: 1) | (SEQ ID NO: 7) | (SEQ ID NO: 18) | (SEQ ID NO: 29) | (SEQ ID NO: 54) | (SEQ ID NO: 71) |
| 8 | ELSMQ | GFDPDDGETMYAEKFQG | SPGYDFFDY | GGNNIGSKSVH | DDSDRPS | QVWDSSSDHLV |
| | (SEQ ID NO: 1) | (SEQ ID NO: 7) | (SEQ ID NO: 18) | (SEQ ID NO: 28) | (SEQ ID NO: 54) | (SEQ ID NO: 73) |
| 9 | ELSMQ | GFDPDDGETMYAEKFQG | SPGYDFFDY | GGNNIGSKSVH | DDRDRPS | QVWDSSNDLLI |
| | (SEQ ID NO: 1) | (SEQ ID NO: 7) | (SEQ ID NO: 18) | (SEQ ID NO: 28) | (SEQ ID NO: 56) | (SEQ ID NO: 71) |
| 10 | ELSMQ | GFDPDDGETMYAEKFQG | SPGYDFFDY | GGNNIGSEGVH | DDSDRPS | QVWDSSNDLLI |
| | (SEQ ID NO: 1) | (SEQ ID NO: 7) | (SEQ ID NO: 18) | (SEQ ID NO: 31) | (SEQ ID NO: 54) | (SEQ ID NO: 71) |
| 11 | ELSMQ | GFDPDDGETMYAEKFQG | SPGYDFFDY | TGTSSDVGGYNYVS | DVSKRPS | QVWDSSNDLLI |
| | (SEQ ID NO: 1) | (SEQ ID NO: 7) | (SEQ ID NO: 18) | (SEQ ID NO: 30) | (SEQ ID NO: 57) | (SEQ ID NO: 71) |
| 12 | ELSMQ | GFDPDGGETMYAEKFQG | SPGYDFFDY | GGNNIGSKSVN | DDSDRPS | QVWDSSNDLLI |
| | (SEQ ID NO: 1) | (SEQ ID NO: 9) | (SEQ ID NO: 18) | (SEQ ID NO: 32) | (SEQ ID NO: 54) | (SEQ ID NO: 71) |
| 13 | ELSMQ | GFDPDDGETMYAEKFQG | SPGYDFFDY | GGNNIGSKSVF | DDSDRPS | QVWDSSSDHLV |
| | (SEQ ID NO: 1) | (SEQ ID NO: 7) | (SEQ ID NO: 18) | (SEQ ID NO: 33) | (SEQ ID NO: 54) | (SEQ ID NO: 73) |
| 14 | ELSMQ | GFDPDDGETMYAEKFQG | SPGYDFFDY | GGYNIGSKFVH | DDRDRPS | QVWDSSNDLLI |
| | (SEQ ID NO: 1) | (SEQ ID NO: 7) | (SEQ ID NO: 18) | (SEQ ID NO: 34) | (SEQ ID NO: 56) | (SEQ ID NO: 71) |
| 15 | ELSMQ | GFDPDDGETMYAEKFQG | SPGYDFFDY | GGDNIGSKSVH | DDSDRPS | QVWDSSNDLLI |
| | (SEQ ID NO: 1) | (SEQ ID NO: 7) | (SEQ ID NO: 18) | (SEQ ID NO: 27) | (SEQ ID NO: 54) | (SEQ ID NO: 71) |
| 16 | ELSMQ | GFDPDDGETMYAEKFQG | SPGYDFFDY | GGNNTGSKSVH | DDSDRPS | QVWDSSNDLLI |
| | (SEQ ID NO: 1) | (SEQ ID NO: 7) | (SEQ ID NO: 18) | (SEQ ID NO: 35) | (SEQ ID NO: 54) | (SEQ ID NO: 71) |
| 17 | ELSMQ | GFDPDDGETMYAEKFQG | SPGYDFFDY | GGSDIGDEIVH | DDSDRPS | QVWDSSSDHLI |
| | (SEQ ID NO: 1) | (SEQ ID NO: 7) | (SEQ ID NO: 18) | (SEQ ID NO: 36) | (SEQ ID NO: 54) | (SEQ ID NO: 72) |
| 18 | ELSMQ | GFDPDDGETMYAEKFQG | SPGYDFFDY | GGNNIGSKSVH | DDSDRPL | QVWDSSNDLLI |
| | (SEQ ID NO: 1) | (SEQ ID NO: 7) | (SEQ ID NO: 18) | (SEQ ID NO: 28) | (SEQ ID NO: 58) | (SEQ ID NO: 71) |
| 19 | ELSMQ | GFDPDDGETMYAEKFQG | SPGYDFFDY | GGSNIGDIRVH | DDSDRPS | QVWDSSNDLLI |
| | (SEQ ID NO: 1) | (SEQ ID NO: 7) | (SEQ ID NO: 18) | (SEQ ID NO: 37) | (SEQ ID NO: 54) | (SEQ ID NO: 71) |
| 20 | ELSMQ | GFDPDDGETMYAEKFQG | SPGYDFFDY | GGNNIGSKGVH | DDSDRPS | QVWDSSNDLLI |
| | (SEQ ID NO: 1) | (SEQ ID NO: 7) | (SEQ ID NO: 18) | (SEQ ID NO: 38) | (SEQ ID NO: 54) | (SEQ ID NO: 71) |
| 21 | ELSMQ | GFDPDDGETMYAEKFQG | SPGYDFFDY | GGNNIGSKSVN | DDSDRPS | QVWDSSNDLLI |
| | (SEQ ID NO: 1) | (SEQ ID NO: 7) | (SEQ ID NO: 18) | (SEQ ID NO: 32) | (SEQ ID NO: 54) | (SEQ ID NO: 71) |
| 22 | ELSMQ | GFDPDDGETMYAEKFQG | SPGYDFFDY | GGNNIGSKNVH | DDSDRPS | QVWDSSNDLLI |
| | (SEQ ID NO: 1) | (SEQ ID NO: 7) | (SEQ ID NO: 18) | (SEQ ID NO: 39) | (SEQ ID NO: 54) | (SEQ ID NO: 71) |
| 23 | ELSMQ | GFDPDDGETMYAEKFQG | SPGYDFFDY | GGNNIGIKSVH | DDSDRPS | QVWDSSNDLLI |
| | (SEQ ID NO: 1) | (SEQ ID NO: 7) | (SEQ ID NO: 18) | (SEQ ID NO: 40) | (SEQ ID NO: 54) | (SEQ ID NO: 71) |
| 24 | ELSMQ | GFDPDDGETMYAEKLQG | SPGYDFFDY | GGNNIGSKSVH | DDSDRPS | QVWDSSNDLLI |
| | (SEQ ID NO: 1) | (SEQ ID NO: 10) | (SEQ ID NO: 18) | (SEQ ID NO: 28) | (SEQ ID NO: 54) | (SEQ ID NO: 71) |
| 25 | ELSMQ | GFDPDDGETMYAEKFQG | SPGYDFFDY | GGNNIGRKAVH | DDSDRPS | QVWDSSNDLLI |
| | (SEQ ID NO: 1) | (SEQ ID NO: 7) | (SEQ ID NO: 18) | (SEQ ID NO: 41) | (SEQ ID NO: 54) | (SEQ ID NO: 71) |
| 26 | ELSMQ | GFDPDDGETMYAEKFQG | SPGYDFFDY | GGNNIGSKSVH | DDSDRPS | QVWDSSIDHLI |
| | (SEQ ID NO: 1) | (SEQ ID NO: 7) | (SEQ ID NO: 18) | (SEQ ID NO: 28) | (SEQ ID NO: 54) | (SEQ ID NO: 70) |
| 27 | ELSMQ | GFDPDDGETMYAEKFQG | SPGYDFFDY | GGNNIGIKSVH | DDRDRPS | QVWDSSNDLLI |
| | (SEQ ID NO: 1) | (SEQ ID NO: 7) | (SEQ ID NO: 18) | (SEQ ID NO: 40) | (SEQ ID NO: 56 | (SEQ ID NO: 71) |
| 28 | ELSMQ | GFDPDDGETMYAEKFQG | SPGYDFFDY | GGNNIGSKSVH | DDSDRPS | QVWDADSDQLI |
| | (SEQ ID NO: 1) | (SEQ ID NO: 7) | (SEQ ID NO: 18) | (SEQ ID NO: 28) | (SEQ ID NO: 54) | (SEQ ID NO: 74) |
| 29 | ELSMQ | GFDPDDGETMYAEKFQG | SPGYDFFDY | GGNNIGSKSVH | DDSDRPS | QVWDSSSDLLI |
| | (SEQ ID NO: 1) | (SEQ ID NO: 7) | (SEQ ID NO: 18) | (SEQ ID NO: 28) | (SEQ ID NO: 54) | (SEQ ID NO: 75) |
| 30 | ELSMQ | GFDPDDGETMYAEKFQG | SPGYDFFDY | GGNNIGTKSVH | DDSDRPS | QVWDSSNDLLI |
| | (SEQ ID NO: 1) | (SEQ ID NO: 7) | (SEQ ID NO: 18) | (SEQ ID NO: 118) | (SEQ ID NO: 54) | (SEQ ID NO: 71) |

TABLE 2
CDRs of mutant clones.

| | Heavy | Light | | | | | | |
|---|---|---|---|---|---|---|---|---|
| Clone | Chain | Chain | | | | | | |
| No. | Mutations | mutations | VH CDR1 | VH CDR2 | VHCDR3 | VL CDR1 | VL CDR2 | VLCDR3 |
| 6 | Parental | Parental | ELSMQ | GFDPDDGETMYAEKFQG | SPGYDFFDY | TGTSSDVGGYNYVS | EVSNRPS | QVWDSSNDLLI |
| | | | (SEQ ID NO: 1) | (SEQ ID NO: 7) | (SEQ ID NO: 18) | (SEQ ID NO: 30) | (SEQ ID NO: 55) | (SEQ ID NO: 71) |
| 55 | N297A | Parental | ELSMQ | GFDPDDGETMYAEKFQG | SPGYDFFDY | TGTSSDVGGYNYVS | EVSNRPS | QVWDSSNDLLI |
| | | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 56 | D54A | Parental | ELSMQ | GFDPDAGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 92) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 57 | D54S | Parental | ELSMQ | GFDPDSGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 93) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 58 | D54E | Parental | ELSMQ | GFDPDEGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 94) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 59 | G55A | Parental | ELSMQ | GFDPDDAETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 95) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 60 | R82aS | Parental | ELSMQ | GFDPDDGETMYAEKFQG | SPGYDFFDY | TGTSSDVGGYNYVS | EVSNRPS | QVWDSSNDLLI |
| | T83R | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | L89V | (mutations | | | | | | |
| | N297A | in heavy | | | | | | |
| | | chain | | | | | | |
| | | only) | | | | | | |
| 61 | D54A | Parental | ELSMQ | GFDPDAGETMYAEKFQG | SPGYDFFDY | TGTSSDVGGYNYVS | EVSNRPS | QVWDSSNDLLI |
| | R82aS | clone 6 | (SEQ ID NO: 1) | (SEQ ID NO: 92) | (SEQ ID NO: 18) | (SEQ ID NO: 30) | (SEQ ID NO: 55) | (SEQ ID NO: 71) |
| | T83R | (mutations | | | | | | |
| | L89V | in heavy | | | | | | |
| | N297A | chain | | | | | | |
| | | only) | | | | | | |
| 62 | D54S | Parental | ELSMQ | GFDPDSGETMYAEKFQG | SPGYDFFDY | TGTSSDVGGYNYVS | EVSNRPS | QVWDSSNDLLI |
| | R82aS | clone 6 | (SEQ ID NO: 1) | (SEQ ID NO: 93) | (SEQ ID NO: 18) | (SEQ ID NO: 30) | (SEQ ID NO: 55) | (SEQ ID NO: 71) |
| | T83R | (mutations | | | | | | |
| | L89V | in heavy | | | | | | |
| | N297A | chain | | | | | | |
| | | only) | | | | | | |
| 63 | D54E | Parental | ELSMQ | GFDPDEGETMYAEKFQG | SPGYDFFDY | TGTSSDVGGYNYVS | EVSNRPS | QVWDSSNDLLI |
| | R82aS | clone 6 | (SEQ ID NO: 1) | (SEQ ID NO: 94) | (SEQ ID NO: 18) | (SEQ ID NO: 30) | (SEQ ID NO: 55) | (SEQ ID NO: 71) |
| | T83R | (mutations | | | | | | |
| | L89V | in heavy | | | | | | |
| | N297A | chain | | | | | | |
| | | only) | | | | | | |
| 64 | G55A | Parental | ELSMQ | GFDPDDAETMYAEKFQG | SPGYDFFDY | TGTSSDVGGYNYVS | EVSNRPS | QVWDSSNDLLI |
| | R82aS | clone 6 | (SEQ ID NO: 1) | (SEQ ID NO: 95) | (SEQ ID NO: 18) | (SEQ ID NO: 30) | (SEQ ID NO: 55) | (SEQ ID NO: 71) |
| | T83R | (mutations | | | | | | |
| | L89V | in heavy | | | | | | |
| | N297A | chain | | | | | | |
| | | only) | | | | | | |
| 65 | N297A | Germline | ELSMQ | GFDPDDGETMYAEKFQG | SPGYDFFDY | TGTSSDVGGYNYVS | EVSNRPS | QVWDSSNDLLI |
| | | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 66 | D54A | Germline | ELSMQ | GFDPDAGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 92) | (SEQ ID NO: 18) | (SEQ ID NO: 30) | (SEQ ID NO: 55) | (SEQ ID NO: 71) |
| | | graft | | | | | | |
| 67 | D54S | Germline | ELSMQ | GFDPDSGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 93) | (SEQ ID NO: 18) | (SEQ ID NO: 30) | (SEQ ID NO: 55) | (SEQ ID NO: 71) |
| | | graft | | | | | | |
| 68 | D54E | Germline | ELSMQ | GFDPDEGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 94) | (SEQ ID NO: 18) | (SEQ ID NO: 30) | (SEQ ID NO: 55) | (SEQ ID NO: 71) |
| | | graft | | | | | | |
| 69 | G55A | Germline | ELSMQ | GFDPDDAETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 95) | (SEQ ID NO: 18) | (SEQ ID NO: 30) | (SEQ ID NO: 55) | (SEQ ID NO: 71) |
| | | graft | | | | | | |
| 70 | R82aS | Germline | ELSMQ | GFDPDDGETMYAEKFQG | SPGYDFFDY | TGTSSDVGGYNYVS | EVSNRPS | QVWDSSNDLLI |
| | T83R | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | L89V | graft | | | | | | |
| | N297A | | | | | | | |
| 71 | D54A | Germline | ELSMQ | GFDPDAGETMYAEKFQG | SPGYDFFDY | TGTSSDVGGYNYVS | EVSNRPS | QVWDSSNDLLI |
| | R82aS | 3-21 CDR | (SEQ ID NO: 1) | (SEQ ID NO: 92) | (SEQ ID NO: 18) | (SEQ ID NO: 30) | (SEQ ID NO: 55) | (SEQ ID NO: 71) |
| | T83R | graft | | | | | | |
| | L89V | | | | | | | |
| | N297A | | | | | | | |
| 72 | D54S | Germline | ELSMQ | GFDPDSGETMYAEKFQG | SPGYDFFDY | TGTSSDVGGYNYVS | EVSNRPS | QVWDSSNDLLI |
| | R82aS | 3-21 CDR | (SEQ ID NO: 1) | (SEQ ID NO: 93) | (SEQ ID NO: 18) | (SEQ ID NO: 30) | (SEQ ID NO: 55) | (SEQ ID NO: 71) |
| | T83R | graft | | | | | | |
| | L89V | | | | | | | |
| | N297A | | | | | | | |
| 73 | D54E | Germline | ELSMQ | GFDPDEGETMYAEKFQG | SPGYDFFDY | TGTSSDVGGYNYVS | EVSNRPS | QVWDSSNDLLI |
| | R82aS | 3-21 CDR | (SEQ ID NO: 1) | (SEQ ID NO: 94) | (SEQ ID NO: 18) | (SEQ ID NO: 30) | (SEQ ID NO: 55) | (SEQ ID NO: 71) |
| | T83R | graft | | | | | | |
| | L89V | | | | | | | |
| | N297A | | | | | | | |
| 74 | G55A | Germline | ELSMQ | GFDPDDAETMYAEKFQG | SPGYDFFDY | TGTSSDVGGYNYVS | EVSNRPS | QVWDSSNDLLI |
| | R82aS | 3-21 CDR | (SEQ ID NO: 1) | (SEQ ID NO: 95) | (SEQ ID NO: 18) | (SEQ ID NO: 30) | (SEQ ID NO: 55) | (SEQ ID NO: 71) |
| | T83R | graft | | | | | | |
| | L89V | | | | | | | |
| | N297A | | | | | | | |
| 75 | N297A | Germline | ELSMQ | GFDPDDGETMYAEKFQG | SPGYDFFDY | GGNNIGSKSVH | DDSDRPS | QVWDSSNDLLI |
| | | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 76 | D54A | Germline | ELSMQ | GFDPDAGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 92) | (SEQ ID NO: 18) | (SEQ ID NO: 28) | (SEQ ID NO: 54) | (SEQ ID NO: 71) |
| | | 6 CDR3 | | | | | | |
| 77 | D54S | Germline | ELSMQ | GFDPDSGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 93) | (SEQ ID NO: 18) | (SEQ ID NO: 28) | (SEQ ID NO: 54) | (SEQ ID NO: 71) |
| | | 6 CDR3 | | | | | | |
| 78 | D54E | Germline | ELSMQ | GFDPDEGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 94) | (SEQ ID NO: 18) | (SEQ ID NO: 28) | (SEQ ID NO: 54) | (SEQ ID NO: 71) |
| | | 6 CDR3 | | | | | | |
| 79 | G55A | Germline | ELSMQ | GFDPDDAETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 95) | (SEQ ID NO: 18) | (SEQ ID NO: 28) | (SEQ ID NO: 54) | (SEQ ID NO: 71) |
| | | 6 CDR3 | | | | | | |
| 80 | R82aS | Germline | ELSMQ | GFDPDDGETMYAEKFQG | SPGYDFFDY | GGNNIGSKSVH | DDSDRPS | QVWDSSNDLLI |
| | T83R | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | L89V | 6 CDR3 | | | | | | |
| | N297A | | | | | | | |
| 81 | D54A | Germline | ELSMQ | GFDPDAGETMYAEKFQG | SPGYDFFDY | GGNNIGSKSVH | DDSDRPS | QVWDSSNDLLI |
| | R82aS | 3-21 clone | (SEQ ID NO: 1) | (SEQ ID NO: 92) | (SEQ ID NO: 18) | (SEQ ID NO: 28) | (SEQ ID NO: 54) | (SEQ ID NO: 71) |
| | T83R | 6 CDR3 | | | | | | |
| | L89V | | | | | | | |
| | N297A | | | | | | | |
| 82 | D54S | Germline | ELSMQ | GFDPDSGETMYAEKFQG | SPGYDFFDY | GGNNIGSKSVH | DDSDRPS | QVWDSSNDLLI |
| | R82aS | 3-21 clone | (SEQ ID NO: 1) | (SEQ ID NO: 93) | (SEQ ID NO: 18) | (SEQ ID NO: 28) | (SEQ ID NO: 54) | (SEQ ID NO: 71) |
| | T83R | 6 CDR3 | | | | | | |
| | L89V | | | | | | | |
| | N297A | | | | | | | |
| 83 | D54E | Germline | ELSMQ | GFDPDEGETMYAEKFQG | SPGYDFFDY | GGNNIGSKSVH | DDSDRPS | QVWDSSNDLLI |
| | R82aS | 3-21 clone | (SEQ ID NO: 1) | (SEQ ID NO: 94) | (SEQ ID NO: 18) | (SEQ ID NO: 28) | (SEQ ID NO: 54) | (SEQ ID NO: 71) |
| | T83R | 6 CDR3 | | | | | | |
| | L89V | | | | | | | |
| | N297A | | | | | | | |
| 84 | G55A | Germline | ELSMQ | GFDPDDAETMYAEKFQG | SPGYDFFDY | GGNNIGSKSVH | DDSDRPS | QVWDSSNDLLI |
| | R82aS | 3-21 clone | (SEQ ID NO: 1) | (SEQ ID NO: 95) | (SEQ ID NO: 18) | (SEQ ID NO: 28) | (SEQ ID NO: 54) | (SEQ ID NO: 71) |
| | T83R | 6 CDR3 | | | | | | |
| | L89V | | | | | | | |
| | N297A | | | | | | | |
| 85 | E1Q | Parental | ELSMQ | GFDPDDGETMYAEKFQG | SPGYDFFDY | TGTSSDVGGYNYVS | EVSNRPS | QVWDSSNDLLI |
| | L67V | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | V69M | (mutations | | | | | | |
| | V78A | in heavy | | | | | | |
| | R82aS | chain | | | | | | |
| | T83R | only) | | | | | | |
| | L89V | | | | | | | |
| | F91Y | | | | | | | |
| | N297A | | | | | | | |
| 86 | D54A | Parental | ELSMQ | GFDPDAGETMYAEKFQG | SPGYDFFDY | TGTSSDVGGYNYVS | EVSNRPS | QVWDSSNDLLI |
| | E1Q | clone 6 | (SEQ ID NO: 1) | (SEQ ID NO: 92) | (SEQ ID NO: 18) | (SEQ ID NO: 30) | (SEQ ID NO: 55) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 87 | D54S | Parental | ELSMQ | GFDPDSGETMYAEKFQG | SPGYDFFDY | TGTSSDVGGYNYVS | EVSNRPS | QVWDSSNDLLI |
| | E1Q | clone 6 | (SEQ ID NO: 1) | (SEQ ID NO: 93) | (SEQ ID NO: 18) | (SEQ ID NO: 30) | (SEQ ID NO: 55) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 88 | D54E | Parental | ELSMQ | GFDPDEGETMYAEKFQG | SPGYDFFDY | TGTSSDVGGYNYVS | EVSNRPS | QVWDSSNDLLI |
| | E1Q | clone 6 | (SEQ ID NO: 1) | (SEQ ID NO: 94) | (SEQ ID NO: 18) | (SEQ ID NO: 30) | (SEQ ID NO: 55) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 89 | G55A | Parental | ELSMQ | GFDPDDAETMYAEKFQG | SPGYDFFDY | TGTSSDVGGYNYVS | EVSNRPS | QVWDSSNDLLI |
| | E1Q | clone 6 | (SEQ ID NO: 1) | (SEQ ID NO: 95) | (SEQ ID NO: 18) | (SEQ ID NO: 30) | (SEQ ID NO: 55) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 90 | E1Q | Germline | ELSMQ | GFDPDDGETMYAEKFQG | SPGYDFFDY | TGTSSDVGGYNYVS | EVSNRPS | QVWDSSNDLLI |
| | L67V | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | V69M | graft | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 91 | D54A | Germline | ELSMQ | GFDPDAGETMYAEKFQG | SPGYDFFDY | TGTSSDVGGYNYVS | EVSNRPS | QVWDSSNDLLI |
| | E1Q | 3-21 CDR | (SEQ ID NO: 1) | (SEQ ID NO: 92) | (SEQ ID NO: 18) | (SEQ ID NO: 30) | (SEQ ID NO: 55) | (SEQ ID NO: 71) |
| | L67V | graft | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 92 | D54S | Germline | ELSMQ | GFDPDSGETMYAEKFQG | SPGYDFFDY | TGTSSDVGGYNYVS | EVSNRPS | QVWDSSNDLLI |
| | E1Q | 3-21 CDR | (SEQ ID NO: 1) | (SEQ ID NO: 93) | (SEQ ID NO: 18) | (SEQ ID NO: 30) | (SEQ ID NO: 55) | (SEQ ID NO: 71) |
| | L67V | graft | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 93 | D54E | Germline | ELSMQ | GFDPDEGETMYAEKFQG | SPGYDFFDY | TGTSSDVGGYNYVS | EVSNRPS | QVWDSSNDLLI |
| | E1Q | 3-21 CDR | (SEQ ID NO: 1) | (SEQ ID NO: 94) | (SEQ ID NO: 18) | (SEQ ID NO: 30) | (SEQ ID NO: 55) | (SEQ ID NO: 71) |
| | L67V | graft | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 94 | G55A | Germline | ELSMQ | GFDPDDAETMYAEKFQG | SPGYDFFDY | TGTSSDVGGYNYVS | EVSNRPS | QVWDSSNDLLI |
| | E1Q | 3-21 CDR | (SEQ ID NO: 1) | (SEQ ID NO: 95) | (SEQ ID NO: 18) | (SEQ ID NO: 30) | (SEQ ID NO: 55) | (SEQ ID NO: 71) |
| | L67V | graft | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 95 | E1Q | Germline | ELSMQ | GFDPDDGETMYAEKFQG | SPGYDFFDY | GGNNIGSKSVH | DDSDRPS | QVWDSSNDLLI |
| | L67V | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | V69M | 6 CDR3 | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 96 | D54A | Germline | ELSMQ | GFDPDAGETMYAEKFQG | SPGYDFFDY | GGNNIGSKSVH | DDSDRPS | QVWDSSNDLLI |
| | E1Q | 3-21 clone | (SEQ ID NO: 1) | (SEQ ID NO: 92) | (SEQ ID NO: 18) | (SEQ ID NO: 28) | (SEQ ID NO: 54) | (SEQ ID NO: 71) |
| | L67V | 6 CDR3 | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 97 | D54S | Germline | ELSMQ | GFDPDSGETMYAEKFQG | SPGYDFFDY | GGNNIGSKSVH | DDSDRPS | QVWDSSNDLLI |
| | E1Q | 3-21 clone | (SEQ ID NO: 1) | (SEQ ID NO: 93) | (SEQ ID NO: 18) | (SEQ ID NO: 28) | (SEQ ID NO: 54) | (SEQ ID NO: 71) |
| | L67V | 6 CDR3 | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 98 | D54E | Germline | ELSMQ | GFDPDEGETMYAEKFQG | SPGYDFFDY | GGNNIGSKSVH | DDSDRPS | QVWDSSNDLLI |
| | E1Q | 3-21 clone | (SEQ ID NO: 1) | (SEQ ID NO: 94) | (SEQ ID NO: 18) | (SEQ ID NO: 28) | (SEQ ID NO: 54) | (SEQ ID NO: 71) |
| | L67V | 6 CDR3 | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 99 | G55A | Germline | ELSMQ | GFDPDDAETMYAEKFQG | SPGYDFFDY | GGNNIGSKSVH | DDSDRPS | QVWDSSNDLLI |
| | E1Q | 3-21 clone | (SEQ ID NO: 1) | (SEQ ID NO: 95) | (SEQ ID NO: 18) | (SEQ ID NO: 28) | (SEQ ID NO: 54) | (SEQ ID NO: 71) |
| | L67V | 6 CDR3 | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 100 | E1Q | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 101 | L67V | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 102 | V69M | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 103 | V78A | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 104 | R82aS | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 105 | T83R | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 106 | L89V | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 107 | F91Y | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 108 | E1Q | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 109 | L67V | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 110 | V69M | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 111 | V78A | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 112 | R82aS | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 113 | T83R | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 114 | L89V | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 115 | F91Y | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 116 | E1Q | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 117 | L67V | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 118 | V69M | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 119 | V78A | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 120 | R82aS | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 121 | T83R | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 122 | L89V | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 123 | F91Y | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |

TABLE 3
Consensus CDR sequences.

| | |
|---|---|
| VH CDR1 | ELSMQ (SEQ ID NO: 1) |
| VH CDR1 | GYTLTELSMQ (SEQ ID NO: 329) |
| VH CDR2 | GFDPDDX₁₀₁ETMYAEX₁₀₂X₁₀₃QG (SEQ ID NO: 102) |
| | X₁₀₁ is D or G; |
| | X₁₀₂ is K or R; and |
| | X₁₀₃ is F or L. |
| VH CDR3 | SPGYDFFDY (SEQ ID NO: 18) |
| VL CDR1 | GGX₁₀₄X₁₀₅X₁₀₆GX₁₀₇X₁₀₈X₁₀₉VX₁₁₀ (SEQ ID |
| | NO: 103) |
| | X₁₀₄ is N, D, or S; |
| | X₁₀₅ is Y, N, or D; |
| | X₁₀₆ is I or T; |
| | X₁₀₇ is S, D, I, R, or T; |
| | X₁₀₈ is K, E, or I; |
| | X₁₀₉ is S, I, R, G, N, or A; and |
| | X₁₁₀ is H, F, or N. |
| VL CDR2 | DDX₁₁₁DRPX₁₁₂ (SEQ ID NO: 104) |
| | X₁₁₁ is G, S, or R; and |
| | X₁₁₂ is S or L. |
| VL CDR3 | QVWDX₁₁₃X₁₁₄X₁₁₅DX₁₁₆X₁₁₇X₁₁₈ (SEQ ID NO: 105) |
| | X₁₁₃ is S or A; |
| | X₁₁₄ is I or S; |
| | X₁₁₅ is S, I, or N; |
| | X₁₁₆ is H, L, or Q; |
| | X₁₁₇ is V or L; and |
| | X₁₁₈ is V or I. |
| VL CDR1 | TGTSSDVGGYNYVS (SEQ ID NO: 30) |
| VL CDR2 | X₁₁₉VSX₁₂₀RPS (SEQ ID NO: 106) |
| | X₁₁₉ is E or D; and |
| | X₁₂₀ is N or K |
| VL CDR3 | QVWDSSNDLLI (SEQ ID NO: 71) |

TABLE 4
Heavy chain, heavy chain variable region, light chain, and light chain variable region sequences.

| Clone | | | | |
|---|---|---|---|---|
| No. | Heavy Chain | VH | Light Chain | VL |
| 1 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNY |
| | LSMQWVRQAPGKGLEWMGGFDPDDGETMYAE | TLTELSMQWVRQAPGKGLEWMGGFDPD | YIGSKSVHWYQQKPGQAPVLVVYDD | IGSKSVHWYQQKPGQAPVLVVYDDGD |
| | KFQGRLTVTEDTSTDTVYMELRSLTSEDTAL | DGETMYAEKFQGRLTVTEDTSTDTVYM | GDRPSGIPARFSGSNSGNTATLTIS | RPSGIPARFSGSNSGNTATLTISRVE |
| | YFCATSPGYDFFDYWGQGTLVTVSSASTKGP | ELRSLTSEDTALYFCATSPGYDFFDYW | RVEAGDEADYYCQVWDSISDHVVFG | AGDEADYYCQVWDSISDHVVFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 264) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 209) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 2 | EVQLVQSGAEVKKPGASVKVSCKVSGYTLTE | EVQLVQSGAEVKKPGASVKVSCKVSGY | SYELTQPPSVSVAPGKTARITCGGD | SYELTQPPSVSVAPGKTARITCGGDN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | YFCATSPGYDFFDYWGQGTLVTVSSASTKGP | ELRSLTSEDTALYFCATSPGYDFFDYW | RVEAGDEADYYCQVWDSSIDHLIFG | AGDEADYYCQVWDSSIDHLIFGGGTK |
| | SVFPLAPSSKSTSGGTAALGCLVKDYFPEPV | GQGTLVTVSS (SEQ ID NO: 165) | GGTKVTVLGQPKAAPSVTLFPPSSE | VTVL (SEQ ID NO: 265) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 210) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 3 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | RFQGRLTVTEDTSTDTVYMELRSLTSEDTAL | DGETMYAERFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 166) | GGTKLTVLGQPKAAPSVTLFPPSSE | LTVL (SEQ ID NO: 266) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 211) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 120) | | | |
| 4 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 266) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 211) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 5 | EVQLVQSGAEVKKPGASVKVSCKVSGYTLTE | EVQLVQSGAEVKKPGASVKVSCKVSGY | SYVLTQPPSVSVAPGQTASITCVGT | SYVLTQPPSVSVAPGQTASITCVGTN |
| | LSMQWVRQAPGKGLEWMGGFDPDDGETMYAE | TLTELSMQWVRQAPGKGLEWMGGFDPD | NFGSKNVHWYQQRPGQAPVLVVYDD | FGSKNVHWYQQRPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | YFCATSPGYDFFDYWGQGTLVTVSSASTKGP | ELRSLTSEDTALYFCATSPGYDFFDYW | RVEAGDEADYYCQVWDSSSDHLIFG | AGDEADYYCQVWDSSSDHLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 267) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 212) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 6 | EVQLVQSGAEVKKPGASVKVSCKVSGYTLTE | EVQLVQSGAEVKKPGASVKVSCKVSGY | QSALTQPASVSGSPGQSITISCTGT | QSALTQPASVSGSPGQSITISCTGTS |
| | LSMQWVRQAPGKGLEWMGGFDPDDGETMYAE | TLTELSMQWVRQAPGKGLEWMGGFDPD | SSDVGGYNYVSWYQQHPGKAPKLMI | SDVGGYNYVSWYQQHPGKAPKLMIYE |
| | KFQGRLTVTEDTSTDTVYMELRSLTSEDTAL | DGETMYAEKFQGRLTVTEDTSTDTVYM | YEVSNRPSGVSNRFSGSNSGNTATL | VSNRPSGVSNRFSGSNSGNTATLTIN |
| | YFCATSPGYDFFDYWGQGTLVTVSSASTKGP | ELRSLTSEDTALYFCATSPGYDFFDYW | TINRVEAGDEADYYCQVWDSSNDLL | RVEAGDEADYYCQVWDSSNDLLIFGG |
| | SVFPLAPSSKSTSGGTAALGCLVKDYFPEPV | GQGTLVTVSS (SEQ ID NO: 165) | IFGGGTKLTVLGQPKAAPSVTLFPP | GTKLTVL (SEQ ID NO: 268) |
| | TVSWNSGALTSGVHTFPAVLQSSGLYSLSSV | | SSEELQANKATLVCLISDFYPGAVT | |
| | VTVPSSSLGTQTYICNVNHKPSNTKVDKRVE | | VAWKADSSPVKAGVETTTPSKQSNN | |
| | PKSCDKTHTCPPCPAPELLGGPSVFLFPPKP | | KYAASSYLSLTPEQWKSHRSYSCQV | |
| | KDTLMISRTPEVTCVVVDVSHEDPEVKFNWY | | THEGSTVEKTVAPTECS (SEQ ID | |
| | VDGVEVHNAKTKPREEQYNSTYRVVSVLTVL | | NO: 213) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 7 | EVQLVQSGAEVKKPGASVKVSCKVSGYTLTE | EVQLVQSGAEVKKPGASVKVSCKVSGY | SYVLTQPPSVSVAPGQTASITCVGT | SYVLTQPPSVSVAPGQTASITCVGTN |
| | LSMQWVRQAPGKGLEWMGGFDPDDGETMYAE | TLTELSMQWVRQAPGKGLEWMGGFDPD | NFGSKNVHWYQQRPGQAPVLVVYDD | FGSKNVHWYQQRPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 269) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 214) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 8 | QVQLVQSGAEVKKPGSSVKVSCKVSGYTLTE | QVQLVQSGAEVKKPGSSVKVSCKVSGY | SYELTQPPSVSVAPGQTARITCGGN | SYELTQPPSVSVAPGQTARITCGGNN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGRTATLTIS | RPSGIPERFSGSNSGRTATLTISRVE |
| | YFCATSPGYDFFDYWGQGTLVTVSSASTKGP | ELRSLTSEDTALYFCATSPGYDFFDYW | RVEAGDEADYHCQVWDSSSDHLVFG | AGDEADYHCQVWDSSSDHLVFGGGTK |
| | SVFPLAPSSKSTSGGTAALGCLVKDYFPEPV | GQGTLVTVSS (SEQ ID NO: 167) | GGTKLTVLGQPKAAPSVTLFPPSSE | LTVL (SEQ ID NO: 270) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 215) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 121) | | | |
| 9 | EVQLVQSGAEVKKPGASVKVSCKVSGYTLTE | EVQLVQSGAEVKKPGASVKVSCKVSGY | SYVLTQPPSVSVAPGQTARIPCGGN | SYVLTQPPSVSVAPGQTARIPCGGNN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDRD |
| | KFQGRLTVTEDTSTDTVYMELRSLTSEDTAL | DGETMYAEKFQGRLTVTEDTSTDTVYM | RDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 271) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 216) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 10 | EVQLVQSGAEVKKPGASVKVSCKVSGYTLTE | EVQLVQSGAEVKKPGASVKVSCKVSGY | SYELTQPPSVSVAPGQTARIPCGGN | SYELTQPPSVSVAPGQTARIPCGGNN |
| | LSMQWVRQAPGKGLEWMGGFDPDDGETMYAE | TLTELSMQWVRQAPGKGLEWMGGFDPD | NIGSEGVHWYQQKPGQAPVLVVYDD | IGSEGVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 272) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 217) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 11 | EVRLVQSGAEVKKPGASVKVSCKVSGYTLTE | EVRLVQSGAEVKKPGASVKVSCKVSGY | QSALTQPRSVSGSPGQSVTISCTGT | QSALTQPRSVSGSPGQSVTISCTGTS |
| | LSMQWVRQAPGKGLEWMGGFDPDDGETMYAE | TLTELSMQWVRQAPGKGLEWMGGFDPD | SSDVGGYNYVSWYQQHPGKAPKLMI | SDVGGYNYVSWYQQHPGKAPKLMIYD |
| | KFQGRLTVTEDTSTDTVYMELRSLTSEDTAL | DGETMYAEKFQGRLTVTEDTSTDTVYM | YDVSKRPSGVPDRFSGSNSGNTATL | VSKRPSGVPDRFSGSNSGNTATLTIN |
| | YFCATSPGYDFFDYWGQGTLVTVSSASTKGP | ELRSLTSEDTALYFCATSPGYDFFDYW | TINRVEAGDEADYYCQVWDSSNDLL | RVEAGDEADYYCQVWDSSNDLLIFGG |
| | SVFPLAPSSKSTSGGTAALGCLVKDYFPEPV | GQGTLVTVSS (SEQ ID NO: 168) | IFGGGTKLTVLGQPKAAPSVTLFPP | GTKLTVL (SEQ ID NO: 273) |
| | TVSWNSGALTSGVHTFPAVLQSSGLYSLSSV | | SSEELQANKATLVCLISDFYPGAVT | |
| | VTVPSSSLGTQTYICNVNHKPSNTKVDKRVE | | VAWKADSSPVKAGVETTTPSKQSNN | |
| | PKSCDKTHTCPPCPAPELLGGPSVFLFPPKP | | KYAASSYLSLTPEQWKSHRSYSCQV | |
| | KDTLMISRTPEVTCVVVDVSHEDPEVKFNWY | | THEGSTVEKTVAPTECS (SEQ ID | |
| | VDGVEVHNAKTKPREEQYNSTYRVVSVLTVL | | NO: 218) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 122) | | | |
| 12 | EVQLVQSGAEVKKPGASVKVSCKVSGYTLTE | EVQLVQSGAEVKKPGASVKVSCKVSGY | SYVLTQPPSVSVAPGQTARVSCGGN | SYVLTQPPSVSVAPGQTARVSCGGNN |
| | LSMQWVRQAPGKGLEWMGGFDPDGGETMYAE | TLTELSMQWVRQAPGKGLEWMGGFDPD | NIGSKSVNWYRQKPGQAPVLVIYDD | IGSKSVNWYRQKPGQAPVLVIYDDSD |
| | KFQGRLTVTEDTSTDTVYMELRSLTSEDTAL | GGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 169) | GGTKLTVLGQPKAAPSVTLFPPSSE | LTVL (SEQ ID NO: 274) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 219) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 123) | | | |
| 13 | QVQLVQSGAEVKKPGASVKVSCKVSGYTLTE | QVQLVQSGAEVKKPGASVKVSCKVSGY | SYVLTQPPSVSVAPGKTARITCGGN | SYVLTQPPSVSVAPGKTARITCGGNN |
| | LSMQWVRQAPGKGLEWMGGFDPDDGETMYAE | TLTELSMQWVRQAPGKGLEWMGGFDPD | NIGSKSVFWYQQKPGQAPVLVVYDD | IGSKSVFWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISGTQ |
| | YFCATSPGYDFFDYWGQGTLVTVSSASTKGP | ELRSLTSEDTALYFCATSPGYDFFDYW | GTQAMDEADYYCQVWDSSSDHLVFG | AMDEADYYCQVWDSSSDHLVFGGGTK |
| | SVFPLAPSSKSTSGGTAALGCLVKDYFPEPV | GQGTLVTVSS (SEQ ID NO: 170) | GGTKLTVLGQPKAAPSVTLFPPSSE | LTVL (SEQ ID NO: 275) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 220) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 124) | | | |
| 14 | EVQLVQSGAEVKKPGASVKVSCKVSGYTLTE | EVQLVQSGAEVKKPGASVKVSCKVSGY | SYVLTQPPSVSVAPGKTARITCGGY | SYVLTQPPSVSVAPGKTARITCGGYN |
| | LSMQWVRQAPGKGLEWMGGFDPDDGETMYAE | TLTELSMQWVRQAPGKGLEWMGGFDPD | NIGSKFVHWYRQRPGQAPVLVVYDD | IGSKFVHWYRQRPGQAPVLVVYDDRD |
| | KFQGRLAVTEDTSTDTVYMELRSLTSEDTAL | DGETMYAEKFQGRLAVTEDTSTDTVYM | RDRPSGIPERFSGSNSGNTATLTIS | RPSGIPERFSGSNSGNTATLTISRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 171) | GGTKLTVLGQPKAAPSVTLFPPSSE | LTVL (SEQ ID NO: 276) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 221) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 125) | | | |
| 15 | EVQLVQSGAEVKKPGASVKVSCKVSGYTLTE | EVQLVQSGAEVKKPGASVKVSCKVSGY | SYELTQPPSVSVAPGKTARITCGGD | SYELTQPPSVSVAPGKTARITCGGDN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 277) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 222) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 16 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | LSMQWVRQAPGKGLEWMGGFDPDDGETMYAE | TLTELSMQWVRQAPGKGLEWMGGFDPD | NTGSKSVHWYQQKPGQAPVLVVYDD | TGSKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 278) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 223) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 17 | EVQLVQSGAEVKKPGASVKVSCKVSGYTLTE | EVQLVQSGAEVKKPGASVKVSCKVSGY | SYVLTQPPSVSVAPGQTARITCGGS | SYVLTQPPSVSVAPGQTARITCGGSD |
| | LSMQWVRQAPGKGLEWMGGFDPDDGETMYAE | TLTELSMQWVRQAPGKGLEWMGGFDPD | DIGDEIVHWYQQKPGQAPVLVVYDD | IGDEIVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRAQ |
| | YFCATSPGYDFFDYWGQGTLVTVSSASTKGP | ELRSLTSEDTALYFCATSPGYDFFDYW | RAQAGDEADYYCQVWDSSSDHLIFG | AGDEADYYCQVWDSSSDHLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 279) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 224) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 18 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | KFQGRLTVTEDTSTDTVYMELRSLTSEDTAL | DGETMYAEKFQGRLTVTEDTSTDTVYM | SDRPLGIPERFSGSNSGNTATLTIN | RPLGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 280) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 225) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 19 | EVQLVQSGAEVKKPGASVKVSCKVSGYTLTE | EVQLVQSGAEVKKPGASVKVSCKVSGY | SYVLTQPLSVSVALGQTARITCGGS | SYVLTQPLSVSVALGQTARITCGGSN |
| | LSMQWVRQAPGKGLEWMGGFDPDDGETMYAE | TLTELSMQWVRQAPGKGLEWMGGFDPD | NIGDIRVHWYQQKPGQAPVLVVYDD | IGDIRVHWYQQKPGQAPVLVVYDDSD |
| | KFQGRLTVTEDTPTDTVYMELRSLTSEDTAL | DGETMYAEKFQGRLTVTEDTPTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 172) | GGTKLTVLGQPKAAPSVTLFPPSSE | LTVL (SEQ ID NO: 281) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 226) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 126) | | | |
| 20 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | LSMQWVRQAPGKGLEWMGGFDPDDGETMYAE | TLTELSMQWVRQAPGKGLEWMGGFDPD | NIGSKGVHWYQQKPGQAPVLVVYDD | IGSKGVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 282) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 227) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 21 | EVQLVQSGAEVKKPGASVKVSCEVSGYTLTE | EVQLVQSGAEVKKPGASVKVSCEVSGY | SYVLTQPPSVSVAPGQTARVSCGGN | SYVLTQPPSVSVAPGQTARVSCGGNN |
| | LSMQWVRQAPGKGLEWMGGFDPDDGETMYAE | TLTELSMQWVRQAPGKGLEWMGGFDPD | NIGSKSVNWYRQKPGQAPVLVIYDD | IGSKSVNWYRQKPGQAPVLVIYDDSD |
| | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 173) | GGTKLTVLGQPKAAPSVTLFPPSSE | LTVL (SEQ ID NO: 283) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 228) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 127) | | | |
| 22 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | LSMQWVRQAPGKGLEWMGGFDPDDGETMYAE | TLTELSMQWVRQAPGKGLEWMGGFDPD | NIGSKNVHWYQQKPGQAPVLVVYDD | IGSKNVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 284) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 229) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 23 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | LSMQWVRQAPGKGLEWMGGFDPDDGETMYAE | TLTELSMQWVRQAPGKGLEWMGGFDPD | NIGIKSVHWYQQKPGQAPVLVVYDD | IGIKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 285) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 230) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 24 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | KLQGRLTVTEDTSTDTVYMELRSLTSEDTAL | DGETMYAEKLQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 174) | GGTKLTVLGQPKAAPSVTLFPPSSE | LTVL (SEQ ID NO: 286) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 231) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 128) | | | |
| 25 | EVQLVQSGAEVKKPGASVKVSCKVSGYTLTE | EVQLVQSGAEVKKPGASVKVSCKVSGY | SYELTQPPSVAVAPGQTARITCGGN | SYELTQPPSVAVAPGQTARITCGGNN |
| | LSMQWVRQAPGKGLEWMGGFDPDDGETMYAE | TLTELSMQWVRQAPGKGLEWMGGFDPD | NIGRKAVHWYQQKPGQAPVLVVYDD | IGRKAVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 287) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 232) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 26 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | YFCATSPGYDFFDYWGQGTLVTVSSASTKGP | ELRSLTSEDTALYFCATSPGYDFFDYW | RVEAGDEADYYCQVWDSSIDHLIFG | AGDEADYYCQVWDSSIDHLIFGGGTK |
| | SVFPLAPSSKSTSGGTAALGCLVKDYFPEPV | GQGTLVTVSS (SEQ ID NO: 165) | GGTKVTVLGQPKAAPSVTLFPPSSE | VTVL (SEQ ID NO: 288) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 233) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 27 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | LSMQWVRQAPGKGLEWMGGFDPDDGETMYAE | TLTELSMQWVRQAPGKGLEWMGGFDPD | NIGIKSVHWYQQKPGQAPVLVVYDD | IGIKSVHWYQQKPGQAPVLVVYDDRD |
| | KFQGRLTVTEDTSTDTVYMELRSLTSEDTAL | DGETMYAEKFQGRLTVTEDTSTDTVYM | RDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 289) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 234) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 28 | EVQLVQSGAEVKKPGASVKVSCKVSGYTLTE | EVQLVQSGAEVKKPGASVKVSCKVSGY | SYELTQPPSVSVAPGQTARITCGGN | SYELTQPPSVSVAPGQTARITCGGNN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | YFCATSPGYDFFDYWGQGTLVTVSSASTKGP | ELRSLTSEDTALYFCATSPGYDFFDYW | RVEAGDEADYYCQVWDADSDQLIFG | AGDEADYYCQVWDADSDQLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 290) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 235) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 29 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | YFCATSPGYDFFDYWGQGTLVTVSSASTKGP | ELRSLTSEDTALYFCATSPGYDFFDYW | RVEAGDEADYYCQVWDSSSDLLIFG | AGDEADYYCQVWDSSSDLLIFGGGAK |
| | SVFPLAPSSKSTSGGTAALGCLVKDYFPEPV | GQGTLVTVSS (SEQ ID NO: 165) | GGAKLTVLGQPKAAPSVTLFPPSSE | LTVL (SEQ ID NO: 291) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 236) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |
| 30 | EVQLVQSGAEVKKPGASVKVSCKVSGYTLTE | EVQLVQSGAEVKKPGASVKVSCKVSGY | SYVLTQPPSVSVAPGKTATITCGGN | SYVLTQPPSVSVAPGKTATITCGGNN |
| | LSMQWVRQAPGKGLEWMGGFDPDDGETMYAE | TLTELSMQWVRQAPGKGLEWMGGFDPD | NIGTKSVHWYQQKPGQAPVLVVYDD | IGTKSVHWYQQKPGQAPVLVVYDDSD |
| | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 292) |
| | TVSWNSGALTSGVHTFPAVLQSSGLYSLSSV | | ELQANKATLVCLISDFYPGAVTVAW | |
| | VTVPSSSLGTQTYICNVNHKPSNTKVDKRVE | | KADSSPVKAGVETTTPSKQSNNKYA | |
| | PKSCDKTHTCPPCPAPELLGGPSVFLFPPKP | | ASSYLSLTPEQWKSHRSYSCQVTHE | |
| | KDTLMISRTPEVTCVVVDVSHEDPEVKFNWY | | GSTVEKTVAPTECS (SEQ ID | |
| | VDGVEVHNAKTKPREEQYNSTYRVVSVLTVL | | NO: 237) | |
| | HQDWLNGKEYKCKVSNKALPAPIEKTISKAK | | | |
| | GQPREPQVYTLPPSREEMTKNQVSLTCLVKG | | | |
| | FYPSDIAVEWESNGQPENNYKTTPPVLDSDG | | | |
| | SFFLYSKLTVDKSRWQQGNVFSCSVMHEALH | | | |
| | NHYTQKSLSLSPG (SEQ ID NO: 119) | | | |

TABLE 5
Heavy chain, heavy chain variable region, light chain, and light chain variable region sequences of Clone 6 and variants
thereof.

| Clone | | | | |
|---|---|---|---|---|
| No. | Heavy chain sequence | VH | Light chain sequence | VL |
| 6 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 165) | TLFPPSSEELQANKATLVCLISDF | NO: 268) |
| | VDVSHEDPEVKFNWYVDGVEVHNAKTKPREEQYNSTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 119) | | | |
| 55 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 165) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 142) | | | |
| 56 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDAGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDAGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 188) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 143) | | | |
| 57 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDSGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDSGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 189) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 144) | | | |
| 58 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDEGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDEGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 190) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 145) | | | |
| 59 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDAETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDAETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 191) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 146) | | | |
| 60 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 192) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 147) | | | |
| 61 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDAGETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDAGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 193) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 148) | | | |
| 62 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDSGETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDSGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 194) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 149) | | | |
| 63 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDEGETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDEGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 195) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 150) | | | |
| 64 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDAETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDDAETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 196) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 151) | | | |
| 65 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 165) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 142) | | | |
| 66 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDAGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDAGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 188) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 143) | | | |
| 67 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDSGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDSGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 189) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 144) | | | |
| 68 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDEGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDEGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 190) | TLFPPSSEELQANKATLVCLISDF | NO: 315) |
| | VDVSHEDPEVKENWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 145) | | | |
| 69 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDAETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDAETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 191) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 146) | | | |
| 70 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 192) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 147) | | | |
| 71 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDAGETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDAGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 193) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 148) | | | |
| 72 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDSGETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDSGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 194) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 149) | | | |
| 73 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDEGETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDEGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 195) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 150) | | | |
| 74 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDAETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDDAETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 196) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 151) | | | |
| 75 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 165) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 142) | | | |
| 76 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDAGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 188) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 143) | | | |
| 77 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDSGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 189) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 144) | | | |
| 78 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDEGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 190) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 145) | | | |
| 79 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDAETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 191) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 146) | | | |
| 80 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 192) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 147) | | | |
| 81 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDAGETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 193) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 148) | | | |
| 82 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDSGETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 194) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 149) | | | |
| 83 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDEGETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 195) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 150) | | | |
| 84 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDAETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 196) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 151) | | | |
| 85 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDDGETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 197) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 152) | | | |
| 86 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDAGETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDAGETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 198) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 153) | | | |
| 87 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDSGETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDSGETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 199) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 154) | | | |
| 88 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDEGETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDEGETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 200) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 155) | | | |
| 89 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDDAETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDDAETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 201) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 156) | | | |
| 90 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDDGETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 197) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 152) | | | |
| 91 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDAGETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDAGETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 198) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 153) | | | |
| 92 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDSGETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDSGETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 199) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 154) | | | |
| 93 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDEGETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDEGETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 200) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 155) | | | |
| 94 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDDAETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDDAETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 201) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 156) | | | |
| 95 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDDGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 197) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 152) | | | |
| 96 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDAGETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDAGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 198) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 153) | | | |
| 97 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDSGETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDSGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 199) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 154) | | | |
| 98 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDEGETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDEGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 200) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 155) | | | |
| 99 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDDAETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDDAETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 201) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 156) | | | |
| 100 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 170) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 157) | | | |
| 101 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRVTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRVTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 202) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 158) | | | |
| 102 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLIMTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 203) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 159) | | | |
| 103 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTAYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 204) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 160) | | | |
| 104 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELSSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 205) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 161) | | | |
| 105 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLRS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLRSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 206) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 162) | | | |
| 106 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 207) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 163) | | | |
| 107 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYYCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 208) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 164) | | | |
| 108 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 170) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 157) | | | |
| 109 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRVTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRVTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 202) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 158) | | | |
| 110 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTMTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 203) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 159) | | | |
| 111 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTAYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 204) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 160) | | | |
| 112 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELSSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 205) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 161) | | | |
| 113 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLRS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLRSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 206) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 162) | | | |
| 114 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 207) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 163) | | | |
| 115 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYYCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 208) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 164) | | | |
| 116 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 170) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 157) | | | |
| 117 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRVTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRVTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 202) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 158) | | | |
| 118 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTMTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 203) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 159) | | | |
| 119 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTAYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATI | DDSDRPSGIPERFSGSNSGNTATL |
| | SKSTSGGTAALGCLVKDYFPEPVTVSWNSGALTSGVHTFPAVLQ | DTSTDTAYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 204) | PPSSEELQANKATLVCLISDFYPG | NO: 316) |
| | VDVSHEDPEVKENWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 160) | | | |
| 120 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELSSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 205) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 161) | | | |
| 121 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLRS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLRSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 206) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 162) | | | |
| 122 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 207) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 163) | | | |
| 123 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYYCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 208) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 164) | | | |

In some embodiments, an anti-mutCALR antibody comprises a heavy chain CDR1, CDR2, and CDR3 and/or a light chain variable region CDR1, CDR2, and CDR3 from an antibody described herein. In some embodiments, an anti-mutCALR antibody comprises a heavy chain CDR1, CDR2, and CDR3 and a light chain CDR1, CDR2, and CDR3 from an antibody described herein.

In some embodiments, the anti-mutCALR antibody comprises a variant of an anti-mutCALR antibody described herein which comprises one to thirty conservative amino acid substitution(s), e.g., one to twenty-five, one to twenty, one to fifteen, one to ten, one to five, or one to three conservative amino acid substitution(s). In some embodiments, the conservative amino acid substitution(s) is in a CDR of the antibody. In some embodiments, the conservative amino acid substitution(s) is not in a CDR of the antibody. In some embodiments, the conservative amino acid substitution(s) is in a framework region of the antibody.

In some embodiments, an anti-mutCALR antibody comprises a heavy chain variable region CDR1, a heavy chain variable region CDR2, and a heavy chain variable region CDR3, each of which correspond to the heavy chain variable region CDRs set forth in Tables 1-2 for a single clone, and a light chain variable region CDR1, a light chain variable region VL CDR2, and a light chain variable region VL CDR3, each of which correspond to the VL CDRs set forth in Tables 1-2 for a single clone.

In some embodiments, an anti-mutCALR antibody comprises a heavy chain variable region CDR1, a heavy chain variable region CDR2, a heavy chain variable region CDR3, a light chain variable region CDR1, a light chain variable region CDR2, and a light chain variable region CDR3, each of which correspond to the VH and VL CDRs set forth in Tables 1-2 for a single clone.

In some embodiments, an anti-mutCALR antibody comprises a heavy chain variable region (VH) comprising a VH CDR1; a VH CDR2; and a VH CDR3; wherein the VH CDR1 comprises the amino acid sequence ELSMQ (SEQ ID NO:1); the VH CDR2 comprises the amino acid sequence GFDPDDX₁₀₁ETMYAEX₁₀₂X₁₀₃QG (SEQ ID NO:102); wherein X₁₀₁ is D or G; wherein X₁₀₂ is K or R; and wherein X₁₀₃ is F or L; the VH CDR3 is SPGYDFFDY (SEQ ID NO: 18); wherein the antibody comprises a light chain variable region (VL) comprising a VL CDR1, a VL CDR2, and a VL CDR3, wherein the VL CDR1 comprises the amino acid sequence GGX₁₀₄X₁₀₅X₁₀₆GX₁₀₇X₁₀₈X₁₀₉VX₁₁₀ (SEQ ID NO:103), wherein X₁₀₄ is N, D, or S; wherein X₁₀₅ is Y, N, or D; wherein X₁₀₆ is I or T; wherein X₁₀₇ is S, D, I, R, or T; wherein X₁₀₈ is K, E, or I; wherein X₁₀₉ is S, I, R, G, N, or A; and wherein X₁₁₀ is H, F, or N; the VL CDR2 comprises the amino acid sequence DDX₁₁₁DRPX₁₁₂ (SEQ ID NO:104), wherein X₁₁₁ is G, S, or R; and wherein X₁₁₂ is S or L; and the VL CDR3 comprises the amino acid sequence QVWDX₁₁₃X₁₁₄X₁₁₅DX₁₁₆X₁₁₇X₁₁₈ (SEQ ID NO:105), wherein X₁₁₃ is S or A; wherein X₁₁₄ is I or S; wherein X₁₁₅ is S, I, or N; wherein X₁₁₆ is H, L, or Q; wherein X₁₁₇ is V or L; and wherein X₁₁₈ is V or I.

In some embodiments, an anti-mutCALR antibody comprises a heavy chain variable region (VH) comprising a VH CDR1; a VH CDR2; and a VH CDR3; wherein the VH CDR1 comprises the amino acid sequence ELSMQ (SEQ ID NO:1); the VH CDR2 comprises the amino acid sequence GFDPDDX₁₀₁ETMYAEX₁₀₂X₁₀₃QG (SEQ ID NO:102); wherein X₁₀₁ is D or G; wherein X₁₀₂ is K or R; and wherein X₁₀₃ is F or L; the VH CDR3 is SPGYDFFDY (SEQ ID NO: 18); wherein the antibody comprises a light chain variable region (VL) comprising a VL CDR1, a VL CDR2, and a VL CDR3, wherein the VL CDR1 comprises the amino acid sequence TGTSSDVGGYNYVS (SEQ ID NO:30); the VL CDR2 comprises the amino acid sequence X₁₁₉VSX₁₂₀RPS (SEQ ID NO:106); wherein X₁₁₉ is E or D; and wherein X₁₂₀ is N or K; and the VL CDR3 comprises the amino acid sequence QVWDSSNDLLI (SEQ ID NO:71).

In some embodiments, an anti-mutCALR antibody comprises a heavy chain variable region (VH) comprising a VH CDR1; a VH CDR2; and a VH CDR3; wherein: the VH CDR1 comprises the amino acid sequence GYTLTELSMQ (SEQ ID NO:329); the VH CDR2 comprises the amino acid sequence GFDPDDX₁₀₁ETMYAEX₁₀₂X₁₀₃QG (SEQ ID NO:102); wherein X₁₀₁ is D or G; wherein X₁₀₂ is K or R; and wherein X₁₀₃ is F or L; the VH CDR3 is SPGYDFFDY (SEQ ID NO: 18); wherein the antibody comprises a light chain variable region (VL) comprising a VL CDR1, a VL CDR2, and a VL CDR3, wherein: the VL CDR1 comprises the amino acid sequence TGTSSDVGGYNYVS (SEQ ID NO:30); the VL CDR2 comprises the amino acid sequence X₁₁₉VSX₁₂₀RPS (SEQ ID NO:106); wherein X₁₁₉ is E or D; and wherein X₁₂₀ is N or K; and the VL CDR3 comprises the amino acid sequence QVWDSSNDLLI (SEQ ID NO:71).

In some embodiments, an anti-mutCALR antibody comprises a heavy chain variable region with the C-terminal lysine removed. In some embodiments, an anti-mutCALR antibody comprises a heavy chain variable region comprising an amino acid sequence that has the three VH CDRs of any anti-mutCALR clone disclosed herein and which has at least 75%, 80%, 85%, 90%, 95%, 96%, 97%, 98%, 99%, or 100% identity to any one of the VH sequences set forth in Tables 4-5 and a light chain variable region comprising an amino acid sequence that has the three VL CDRs of any anti-mutCALR clone disclosed herein and which has at least 75%, 80%, 85%, 90%, 95%, 96%, 97%, 98%, 99%, or 100% identity to any one of the VL sequences set forth in Tables 4-5.

In some embodiments, an anti-mutCALR antibody comprises a heavy chain variable region comprising any one of the VH sequences set forth in Tables 4-5. In some embodiments, an anti-mutCALR antibody comprises a light chain variable region comprising any one of the VL sequences set forth in Tables 4-5. In some embodiments, an anti-mutCALR antibody comprises a heavy chain variable region comprising any one of the VH sequences set forth in Tables 4-5 and a light chain variable region comprising any one of the VL sequences set forth in Tables 4-5.

Antibodies described herein may be produced in bacterial or eukaryotic cells. Some antibodies, e.g., Fabs, can be produced in bacterial cells, e.g., E. coli cells. Antibodies can also be produced in eukaryotic cells such as transformed cell lines (e.g., CHO, 293E, COS). In addition, antibodies (e.g., scFvs) can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-mutCALR antibody is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as from the medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

In some embodiments, an anti-mutCALR antibody comprises a modification which modulates (e.g., reduces or increases) the Fc region-mediated effector function, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function.

In certain embodiments, an anti-mutCALR antibody has Fc effector function. In certain embodiments, an anti-mutCALR antibody has enhanced Fc effector function. In certain embodiments, an anti-mutCALR antibody exhibits antibody-dependent cell-mediated cytotoxicity (ADCC). An anti-mutCALR antibody can be engineered to enhance the ADCC activity (for review, see Kubota T et al. Cancer Sci. 2009; 100(9):1566-72). For example, ADCC activity of an antibody can be improved when the antibody itself has a low ADCC activity, by slightly modifying the constant region of the antibody (Junttila T T. et al. Cancer Res. 2010; 70(11):4481-9). Changes are sometimes also made to improve storage or production or to remove C-terminal lysines (Kubota T et al. Cancer Sci. 2009; 100(9):1566-72). Another suitable method to improve ADCC activity of an antibody is by enzymatically interfering with the glycosylation pathway resulting in a reduced fucose (von Horsten H H. et al. Glycobiology. 2010; 20(12):1607-18). Alternatively, or additionally, other suitable methods can be used to achieve ADCC enhancement, for instance including glycoengineering (Kyowa Hakko/Biowa, GlycArt (Roche) and Eureka Therapeutics) and mutagenesis, all of which seek to improve Fc binding to low-affinity activating FcγRIIIa, and/or to reduce binding to the low affinity inhibitory FcγRIIb. In certain embodiments, a binding moiety of the present disclosure exhibits enhanced antibody-dependent cell-mediated cytotoxicity (ADCC). In certain embodiments, a binding moiety of the present disclosure is afucosylated.

In certain embodiments, an anti-mutCALR antibody has reduced Fc effector function. In certain embodiments, an anti-mutCALR antibody exhibits reduced or substantially no complement-dependent cytotoxicity (CDC), antibody-dependent cellular cytotoxicity (ADCC) or antibody-dependent cell phagocytosis (ADCP). In certain embodiments, an anti-mutCALR antibody exhibits reduced or substantially no antibody-dependent cell-mediated cytotoxicity (ADCC). An anti-mutCALR antibody can be engineered to reduce effector function, for example ADCC activity, by any suitable method including removal of glycosylation sites in the Fc region. In certain embodiments, an anti-mutCALR antibody that has a reduced Fc effector function (e.g., a reduced ADCC effector function) comprises an N297A mutation on the heavy chain.

In some embodiments, an anti-mutCALR antibody comprises an IgG1 isotype (e.g., IgG1, IgG2, IgG3 or IgG4). In some embodiments, an anti-mutCALR antibody comprises an IgG1. In some embodiments, an IgG1, IgG2, IgG3 or IgG4 anti-mutCALR antibody has Fc-effector function. In some embodiments, an IgG1, IgG2, IgG3 or IgG4 anti-mutCALR antibody is Fc-effector function null. In some embodiments, an IgG1 anti-mutCALR antibody has Fc-effector function. In some embodiments, an IgG1 anti-mutCALR antibody is Fc-effector function null.

In some instances, the anti-mutCALR antibody comprises an antibody fragment. Fragments of the antibodies described herein (e.g., Fab, Fab′, F(ab′)₂, Facb, and Fv) may be prepared by proteolytic digestion of intact antibodies. For example, antibody fragments can be obtained by treating the whole antibody with an enzyme such as papain, pepsin, or plasmin or the FabRICATOR® (IdeS) recombinant enzyme (Genovis AB) that digests IgG antibodies to produce a homogeneous pool of F(ab′)₂ and Fc/2 fragments. Papain digestion of whole antibodies produces F(ab)₂ or Fab fragments; pepsin digestion of whole antibodies yields F(ab′)₂ or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Plueckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology, 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046.

In some instances, the anti-mutCALR antibody comprises a minibody. Minibodies of anti-mutCALR antibodies include diabodies, single chain (scFv), and single-chain (Fv)₂ (sc(Fv)₂).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see, e.g., Huston et al., Proc. Natl. Acad. Sci. U.S.A, 85:5879-5883 (1988); and Pluckthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The heavy chain variable domain and light chain variable domain in an scFv may be derived from any anti-mutCALR antibody described herein.

An sc(Fv)₂ is a minibody in which two VHs and two VLs are linked by a linker to form a single chain (Hudson, et al., J. Immunol. Methods, (1999) 231: 177-189 (1999)). An sc(Fv)₂ can be prepared, for example, by connecting scFvs with a linker. The sc(Fv)₂ of the present invention include antibodies preferably in which two VHs and two VLs are arranged in the order of VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order.

In some instances, the anti-mutCALR antibody comprises a bispecific antibody. Bispecific antibodies are antibodies that have binding specificities for at least two different epitopes. Exemplary bispecific antibodies may bind to two different epitopes of the mutCALR protein. Other such antibodies may combine a mutCALR binding site with a binding site for another antigen. Bispecific antibodies can be prepared as full length antibodies or low molecular weight forms thereof (e.g., F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

In certain embodiments, the bispecific anti-mutCALR antibody comprises a biparatopic antibody. A biparatopic antibody is antibody which recognizes two non-identical epitopes (overlapping or non-overlapping epitopes) on the same target antigen (e.g., the C-terminal of mutCALR domain). A biparatopic anti-mutCALR antibody can comprise two immunoglobulin heavy chain-light chain pairs or one immunoglobulin heavy chain-light chain pair. In some embodiments, a biparatopic anti-mutCALR antibody comprises one immunoglobulin heavy-chain-light chain pair. In some embodiments, a biparatopic anti-mutCALR antibody comprises a full-length antibody comprising one immunoglobulin heavy-chain-light chain pair.

In some instances, the anti-mutCALR antibody comprises a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

The present disclosure further provides an antibody described herein for use in any of the methods described herein.

The present disclosure further provides use of an antibody described herein for the preparation of a medicament for use in any of the methods described herein.

### Combination Therapies

The methods described herein can further comprise administering one or more additional therapeutic agents. The agents can be combined with the present compounds in a single dosage form, or the agents can be administered simultaneously or sequentially as separate dosage forms.

In some embodiments, the additional therapeutic agent is a JAK inhibitor. In some embodiments, the additional therapeutic agent is a JAK1 inhibitor. In some embodiments, the additional therapeutic agent is a JAK2 inhibitor. In some embodiments, the additional therapeutic agent is a JAK1/2 inhibitor. In some embodiments, the additional therapeutic agent is ruxolitinib. In some embodiments, the additional therapeutic agent is ruxolitinib phosphate.

In some embodiments, methods provided herein comprise administering to the patient a JAK2 V617F inhibitor described herein (e.g., a compound of any of Formulas I-Vc) in combination with an antibody that binds to human mutant calreticulin (CALR) described herein.

### Pharmaceutical Formulations and Dosage Forms

When employed as pharmaceuticals, the compounds of the disclosure (i.e., small molecule inhibitors of JAK2V617F and antibodies that bind to human mutant calreticulin (CALR) as described herein) can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating V617F in tissue samples, including human, and for identifying V617F inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes V617F assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as -CDH₂ (i.e., deuteromethyl), -CD₂H (i.e., dideuteromethyl), or -CD₃ (i.e., trideuteromethyl) being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as -CDH₂, -CD₂H, or -CD₃ being substituted for —CH₃). In some embodiments, the compound includes two or more deuterium atoms.

In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

In some embodiments, the compound provided herein, or a pharmaceutically acceptable salt thereof, comprises at least one deuterium atom.

In some embodiments, the compound provided herein, or a pharmaceutically acceptable salt thereof, comprises two or more deuterium atoms.

In some embodiments, the compound provided herein, or a pharmaceutically acceptable salt thereof, comprises three or more deuterium atoms.

In some embodiments, for a compound provided herein, or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro V617F labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind V617F by monitoring its concentration variation when contacting with V617F, through tracking of the labeling. For example, a test compound (labeled) can be evaluated for its ability to reduce binding of another compound which is known to bind to V617F (i.e., standard compound). Accordingly, the ability of a test compound to compete with the standard compound for binding to V617F directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of clonal hematopoiesis of indeterminate potential (CHIP), CHIP-associated diseases or disorders, or any combination thereof, as described herein, which include one or more containers containing a pharmaceutical composition comprising a therapeutically effective amount of a compound or antibody of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Intermediate A. Methyl ((1R,3R)-3-(6-amino-3-(methyl-d₃)-2-oxo-2,3-dihydro-1H-imidazo[4,5-c]pyridin-1-yl)cyclopentyl-1-d)carbamate

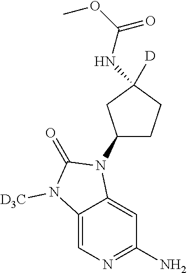 [SMILES: [2H]C([2H])([2H])n1c(=O)n([C@@H]2CC[C@@]([2H])(NC(=O)OC)C2)c2cc(N)ncc21]

### Step 1. tert-Butyl ((1R,3S)-3-hydroxycyclopentyl-3-d)carbamate

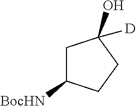 [SMILES: [2H][C@]1(O)CC[C@@H](NC(=O)OC(C)(C)C)C1]

To a flask containing tert-butyl (R)-(3-oxocyclopentyl)carbamate (10.0 g, 50.2 mmol) in MeOH (100 mL) at 0° C. was added sodium borodeuteride (1.26 g, 30.1 mmol) in portions (over 10 times) during 30 min. The reaction mixture was stirred at 0° C. for 3 h. The reaction mixture was concentrated, diluted with DCM (250 mL), and washed with 1 M sodium hydroxide solution. After extraction with DCM (100 mL) for 3 times, the organic layers were combined, dried over MgSO₄ and concentrated in vacuo. A small portion of this crude material was directly used in the synthesis of Intermediate B as a mixture of diastereomers. The rest of the crude material was purified by CombiFlash (earlier peak) to give the desired product as a single isomer (white solid). LCMS calculated for C₆H₁₁DNO₃ (M+H-t-Bu)⁺: m/z=147.1; found 147.1.

### Step 2. tert-Butyl ((1R,3R)-3-(1,3-dioxoisoindolin-2-yl)cyclopentyl-3-d)carbamate

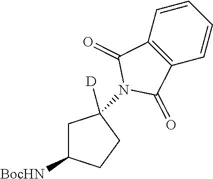 [SMILES: [2H][C@@]1(N2C(=O)c3ccccc3C2=O)CC[C@@H](NC(=O)OC(C)(C)C)C1]

To a flask containing tert-butyl ((1R,3S)-3-hydroxycyclopentyl-3-d)carbamate (3.98 g, 19.7 mmol) in THF (79 ml) was added triphenylphosphine (6.19 g, 23.61 mmol) and isoindoline-1,3-dione (4.34 g, 29.5 mmol). The reaction mixture was cooled down to 0° C. and the diisopropyl (E)-diazene-1,2-dicarboxylate (4.77 g, 4.65 mL, 23.61 mmol) was added dropwise to the flask. The reaction mixture was stirred at 0° C. to r.t for 3 h. The reaction mixture was quenched with water (30 mL) and extraction with DCM. The organic layer was combined, dried over MgSO₄ and concentrated in vacuo. The crude material was purified by Biotage Isolera. LCMS calculated for C₁₈H₂₂DN₂O₄ (M+H)⁺: m/z=332.2; found 332.1.

### Step 3. tert-Butyl ((1R,3R)-3-aminocyclopentyl-3-d)carbamate

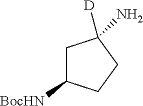 [SMILES: [2H][C@@]1(N)CC[C@@H](NC(=O)OC(C)(C)C)C1]

To a flask containing tert-butyl ((1R,3R)-3-(1,3-dioxoisoindolin-2-yl)cyclopentyl-3-d)carbamate (5.5 g, 16.60 mmol) in ethanol (55.3 mL) was added hydrazine (1.06 g, 1.0 mL, 33.2 mmol). The reaction mixture was stirred at 80° C. for 1.5 h with white solid generation during the reaction. After cooling down to r.t, the reaction mixture was filtered and washed with ethanol (100 mL) and DCM (100 mL) to filter out the white solid as side product. The filtrate was collected and concentrated in vacuo to give the crude product. The obtained crude product was used in the next step without further purification. LCMS calculated for C₆H₁₂DN₂O₂ (M+H-t-Bu)⁺: m/z=146.1; found: 146.1.

### Step 4. Methyl ((1R,3R)-3-aminocyclopentyl-1-d)carbamate

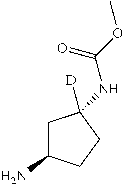

To a flask containing tert-butyl ((1R,3R)-3-aminocyclopentyl-3-d)carbamate (5.0 g, 24.8 mmol) and DIPEA (3.53 g, 4.77 mL, 27.3 mmol) in DCM (200 mL) and MeOH (5 mL) at 0° C. was added methyl chloroformate (1.92 mL, 24.8 mmol) dropwise. After stirring at 0° C. for 10 min., the reaction was quenched with MeOH (10 mL) and concentrated in vacuo. The residue was re-dissolved in DCM (200 mL) and washed with sodium bicarbonate saturated solution, dried over MgSO₄ and concentrated in vacuo. The crude material was treated with 4N HCl in 1,4-dioxane (20 mL). After stirring at r.t for 1 h, the reaction mixture was concentrated in vacuo. The obtained crude product as HCl salt was used in the next step without further purification. LCMS calculated for C₇H₁₄DN₂O₂ (M+H)⁺: m/z=160.1; found: 160.1.

### Step 5. Methyl ((1R,3R)-3-((2-chloro-5-nitropyridin-4-yl)amino)cyclopentyl-1-d)carbamate

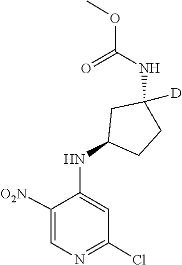 [SMILES: [2H][C@@]1(NC(=O)OC)CC[C@@H](Nc2cc(Cl)ncc2[N+](=O)[O-])C1]

To a vial containing methyl ((1R,3R)-3-aminocyclopentyl-1-d)carbamate (1.60 g, 10.0 mmol) in acetonitrile (1.5 mL) was added DIPEA (4.4 mL, 25.1 mmol) and 2,4-dichloro-5-nitropyridine (1.94 g, 10.0 mmol). The reaction mixture was stirred at 60° C. for 4 h. After cooling down to r.t., the reaction mixture was diluted with water (20 mL) and EtOAc (50 mL). The layers were separated, and the aqueous layer was further extracted with EtOAc (3×50 mL). The combined organic layers were dried over Na₂SO₄, filtered, and concentrated in vacuo. The obtained crude product was used in the next step without further purification. LCMS calculated for C₁₂H₁₅DClN₄O₄ (M+H)⁺: m/z=316.1; found: 316.1.

### Step 6 Methyl ((1R,3R)-3-((5-amino-2-chloropyridin-4-yl)amino)cyclopentyl-1-d)carbamate

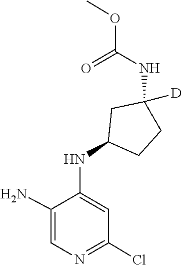 [SMILES: [2H][C@@]1(NC(=O)OC)CC[C@@H](Nc2cc(Cl)ncc2N)C1]

A round-bottom flask containing zinc powder (5.3 g, 80 mmol) and ammonium chloride (4.30 g, 80 mmol) in MeOH (100 mL) was placed in an ice-water bath at 0° C. A solution of tert-butyl ((1R,3R)-3-((2-chloro-5-nitropyridin-4-yl)amino)cyclopentyl-1-d)carbamate (2.8 g, 8.9 mmol) in 30 mL (CH₂Cl₂:MeOH=5:1) was added via addition funnel to the zinc power suspension at the rate of 1 drop/sec with vigorous stirring. After stirring at r.t for 3 h, the reaction mixture was filtered and washed with CH₂Cl₂ (40 mL) for three times. The filtrate was collected and concentrated. The residue was re-dissolved in CH₂Cl₂ (200 mL), washed with water (20 mL) and extracted with CH₂Cl₂. The organic phase was washed with brine, dried over MgSO₄ and concentrated in vacuo. The obtained crude product was used in the next step without further purification. LCMS calculated for C₁₂H₁₇DClN₄O₂ (M+H)⁺: m/z=286.1; found 286.2.

### Step 7. Methyl ((1R,3R)-3-(6-chloro-2-oxo-2,3-dihydro-1H-imidazo[4,5-c]pyridin-1-yl)cyclopentyl-1-d)carbamate

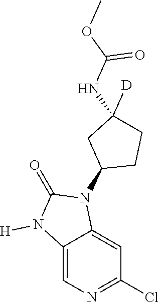 [SMILES: [2H][C@@]1(NC(=O)OC)CC[C@@H](n2c(=O)[nH]c3cnc(Cl)cc32)C1]

To a flask containing methyl ((1R,3R)-3-((5-amino-2-chloropyridin-4-yl)amino)cyclopentyl-1-d)carbamate (2.0 g, 7.0 mmol) in acetonitrile (20 mL) was added bis(2,5-dioxopyrrolidin-1-yl) carbonate (2.0 g, 7.7 mmol) in portions (over 10 times) during 30 min. After stirring at r.t for 16 h, the reaction mixture was concentrated in vacuo and washed with aqueous saturated sodium bicarbonate solution and extracted with CH₂Cl₂. The organic phase was washed with brine, dried over MgSO₄ and concentrated in vacuo. The obtained crude product was used in the next step without further purification. LCMS calculated for C₁₃H₁₅DClN₄O₃ (M+H)⁺: m/z=312.1; found: 312.1.

### Step 8. Methyl ((1R,3R)-3-(6-chloro-3-(methyl-d₃)-2-oxo-2,3-dihydro-1H-imidazo[4,5-c]pyridin-1-yl)cyclopentyl-1-d)carbamate

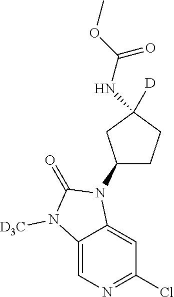 [SMILES: [2H]C([2H])([2H])n1c(=O)n([C@@H]2CC[C@@]([2H])(NC(=O)OC)C2)c2cc(Cl)ncc21]

To a flask containing methyl ((1R,3R)-3-(6-chloro-2-oxo-2,3-dihydro-1H-imidazo[4,5-c]pyridin-1-yl)cyclopentyl-1-d)carbamate (4.9 g, 15.7 mmol) and cesium carbonate (7.7 g, 23.6 mmol) in THF (40 mL) was added iodomethane-d₃ (6.8 g, 2.9 mL, 47.2 mmol). After stirring at r.t for 6 h, the reaction mixture was diluted with CH₂Cl₂ (100 mL), filtered through a Celite pad and washed with CH₂Cl₂ (3×50 mL). The filtrate was washed with aqueous saturated sodium bicarbonate solution, brine, dried over MgSO₄ and concentrated in vacuo. The crude material was purified by Biotage Isolera to afford the title compound. LCMS calculated for C₁₄H₁₄D₄ClN₄O₃ (M+H)⁺: m/z=329.1; found: 329.1.

### Step 9. Methyl ((1R,3R)-3-(6-amino-3-(methyl-d₃)-2-oxo-2,3-dihydro-1H-imidazo[4,5-c]pyridin-1-yl)cyclopentyl-1-d)carbamate

A flask containing methyl ((1R,3R)-3-(6-chloro-3-(methyl-d₃)-2-oxo-2,3-dihydro-1H-imidazo[4,5-c]pyridin-1-yl)cyclopentyl-1-d)carbamate (4.0 g, 12.2 mmol), diphenylmethanimine (4.4 g, 24.3 mmol), tris(dibenzylideneacetone)-dipalladium(0) (1.11 g, 1.22 mmol), 4,5-bis(diphenylphosphino)-9,9-dimethylxanthene (1.41 g, 2.43 mmol) and cesium carbonate (11.9 g, 36.5 mmol) was evacuated and backfilled with nitrogen three times, followed by the addition of 1,4-dioxane (50.0 mL). The flask was heated at 85° C. for 16 h under nitrogen atmosphere. After cooling down to r.t, the reaction was diluted with DCM, filtered through Celite and concentrated in vacuo. The crude material was diluted with MeOH (30 mL) followed by the addition of hydroxylamine hydrochloride (2.4 g, 33.0 mmol) and potassium carbonate (4.5 g, 33 mmol). After stirring at r.t for 1 h, the reaction mixture was filtered through Celite, concentrated in vacuo, purified by Biotage Isolera to give the desired product as white solid. LCMS calculated for C₁₄H₁₆D₄N₅O₃ (M+H)⁺: m/z=310.2; found: 310.2.

### Alternative Synthesis of Intermediate A. Methyl ((1R,3R)-3-(6-amino-3-(methyl-d₃)-2-oxo-2,3-dihydro-1H-imidazo[4,5-c]pyridin-1-yl)cyclopentyl-1-d)carbamate

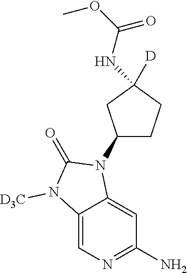 [SMILES: [2H]C([2H])([2H])n1c(=O)n([C@@H]2CC[C@@]([2H])(NC(=O)OC)C2)c2cc(N)ncc21]

### Step 1. tert-Butyl ((1R,3R)-3-((2-chloro-5-nitropyridin-4-yl)amino)cyclopentyl-1-d)carbamate

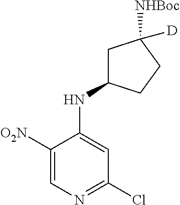 [SMILES: [2H][C@@]1(NC(=O)OC(C)(C)C)CC[C@@H](Nc2cc(Cl)ncc2[N+](=O)[O-])C1]

To a flask containing tert-butyl ((1R,3R)-3-aminocyclopentyl-1-d)carbamate hydrochloride (40.0 g, 168 mmol, PharmaBlock catalog #: PCS4754) and 2,4-dichloro-5-nitropyridine (32.5 g, 168 mmol) in acetonitrile (56.0 ml) was added DIPEA (64.6 mL, 370 mmol). The reaction mixture was stirred at 60° C. for 4 h. After cooling down to r.t., the reaction mixture was diluted with water (250 mL) and filtered. The filter cake was washed with water (100 mL) for 3 times and air dried. The obtained crude product was used in the next step without further purification. LCMS calculated for C₁₅H₂₁DClN₄O₄ (M+H)⁺: m/z=358.1; found 358.1.

### Step 2. tert-Butyl ((1R,3R)-3-((5-amino-2-chloropyridin-4-yl)amino)cyclopentyl-1-d)carbamate

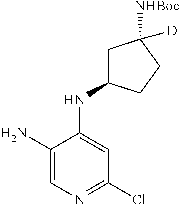 [SMILES: [2H][C@@]1(NC(=O)OC(C)(C)C)CC[C@@H](Nc2cc(Cl)ncc2N)C1]

A round-bottom flask containing zinc powder (22 g, 336 mmol) and ammonium chloride (18 g, 336 mmol) in MeOH (300 mL) was placed in an ice-water bath at 0° C. A solution of tert-butyl ((1R,3R)-3-((2-chloro-5-nitropyridin-4-yl)amino)cyclopentyl-1-d)carbamate (14.7 g, 41.2 mmol) in 120 mL (CH₂Cl₂:MeOH=5:1) was added via addition funnel to the zinc power suspension at the rate of 1 drop/sec with vigorous stirring. After stirring at r.t. for 3 h, the reaction mixture was filtered and washed with CH₂Cl₂ (100 mL) three times. The filtrate was collected and concentrated. The residue was re-dissolved in CH₂Cl₂ (300 mL), washed with water (50 mL), and extracted with CH₂Cl₂. The organic phase was washed with brine, dried over MgSO₄ and concentrated in vacuo. The obtained crude product was used in the next step without further purification. LCMS calculated for C₁₅H₂₃DClN₄O₂ (M+H)⁺: m/z=328.2; found 328.2.

### Step 3. tert-Butyl ((1R,3R)-3-(6-chloro-2-oxo-2,3-dihydro-1H-imidazo[4,5-c]pyridin-1-yl)cyclopentyl-1-d)carbamate

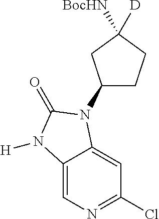 [SMILES: [2H][C@@]1(NC(=O)OC(C)(C)C)CC[C@@H](n2c(=O)[nH]c3cnc(Cl)cc32)C1]

To a flask containing tert-butyl ((1R,3R)-3-((5-amino-2-chloropyridin-4-yl)amino)cyclopentyl-1-d)carbamate (55.0 g, 168 mmol) in acetonitrile (420 mL) was added bis(2,5-dioxopyrrolidin-1-yl) carbonate (47.3 g, 185 mmol) in portions (over 20 times) during 30 min. After stirring at r.t. for 16 h, the reaction mixture was filtered to obtain the solid product as off-white solid. The filtrate was concentrated down to 80 mL in volume, quenched with aqueous saturated sodium carbonate solution and filtered. The solid was combined and washed with acetonitrile (80 mL) for 3 times and air dried. The obtained crude product was used in the next step without further purification. LCMS calculated for C₁₆H₂₁DClN₄O₃ (M+H)⁺: m/z=354.1; found: 354.1.

### Step 4. 1-((1R,3R)-3-Aminocyclopentyl-3-d)-6-chloro-3-(methyl-d₃)-1,3-dihydro-2H-imidazo[4,5-c]pyridin-2-one

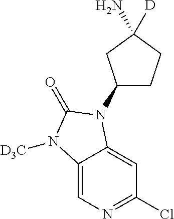 [SMILES: [2H]C([2H])([2H])n1c(=O)n([C@@H]2CC[C@@]([2H])(N)C2)c2cc(Cl)ncc21]

To a flask containing tert-butyl ((1R,3R)-3-(6-chloro-2-oxo-2,3-dihydro-1H-imidazo[4,5-c]pyridin-1-yl)cyclopentyl-1-d)carbamate (55.0 g, 155 mmol) and cesium carbonate (65.8 g, 202 mmol) in THF (390 mL) was added iodomethane-d₃ (45.1 g, 19.3 mL, 311 mmol). After stirring at r.t. for 4 h, the reaction mixture was diluted with CH₂Cl₂ (500 mL), filtered through a celite pad, and washed with CH₂Cl₂ (300 mL) for 3 times. The filtrate was concentrated in vacuo and was washed with aqueous saturated sodium bicarbonate solution. The organic phase was washed with brine, dried over MgSO₄ and concentrated in vacuo. The crude material was dissolved in 4 M HCl in dioxane (400 mL) and MeOH (50 mL). After stirring at 30° C. for 1 h, the reaction mixture was concentrated in vacuo. The obtained crude product as HCl salt of the title compound was used in the next step without further purification. LCMS calculated for C₁₂H₁₂D₄ClN₄O (M+H)⁺: m/z=271.1; found: 271.1.

### Step 5. Methyl ((1R,3R)-3-(6-chloro-3-(methyl-d₃)-2-oxo-2,3-dihydro-1H-imidazo[4,5-c]pyridin-1-yl)cyclopentyl-1-d)carbamate

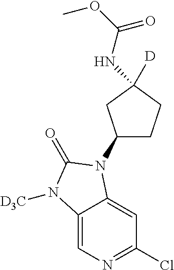 [SMILES: [2H]C([2H])([2H])n1c(=O)n([C@@H]2CC[C@@]([2H])(NC(=O)OC)C2)c2cc(Cl)ncc21]

To a flask containing 1-((1R,3R)-3-aminocyclopentyl-3-d)-6-chloro-3-(methyl-d₃)-1,3-dihydro-2H-imidazo[4,5-c]pyridin-2-one (53.0 g, 154 mmol) and DIPEA (89 mL, 512 mmol) in CH₂Cl₂ (400 mL) and MeOH (20 mL) at 0° C. was added methyl carbonochloridate (16.6 g, 13.6 mL, 176 mmol). After stirring at 0° C. for 5 min, the reaction mixture was quenched with MeOH and concentrated in vacuo. The obtained crude product was re-dissolved in CH₂Cl₂ (750 mL), was washed with brine, dried over MgSO₄ and concentrated in vacuo. The product was purified by washing with 20 mL (CH₂Cl₂:MeOH=20:1) for 3 times to give the desired product as white solid after air drying. LCMS calculated for C₁₄H₁₄D₄ClN₄O₃ (M+H)⁺: m/z=329.1; found: 329.1.

### Step 6 Methyl ((1R,3R)-3-(6-amino-3-(methyl-d₃)-2-oxo-2,3-dihydro-1H-imidazo[4,5-c]pyridin-1-yl)cyclopentyl-1-d)carbamate

A flask containing methyl ((1R,3R)-3-(6-chloro-3-(methyl-d₃)-2-oxo-2,3-dihydro-1H-imidazo[4,5-c]pyridin-1-yl)cyclopentyl-1-d)carbamate (4.0 g, 12.2 mmol), diphenylmethanimine (4.4 g, 24.3 mmol), tris(dibenzylideneacetone)-dipalladium(0) (1.11 g, 1.22 mmol), 4,5-bis(diphenylphosphino)-9,9-dimethylxanthene (1.41 g, 2.43 mmol) and cesium carbonate (11.9 g, 36.5 mmol) was evacuated and backfilled with nitrogen three times, followed by the addition of 1,4-dioxane (50.0 mL). The flask was heated at 85° C. for 16 h under nitrogen atmosphere. After cooling down to r.t., the reaction was diluted with DCM, filtered through Celite and concentrated in vacuo. The crude material was diluted with MeOH (30 mL) followed by the addition of hydroxylamine hydrochloride (2.4 g, 33.0 mmol) and potassium carbonate (4.5 g, 33 mmol). After stirring at r.t for 1 h, the reaction mixture was filtered through Celite, concentrated in vacuo, and purified by Biotage Isolera to give the desired product as white solid. LCMS calculated for C₁₄H₁₆D₄N₅O₃ (M+H)⁺: m/z=310.2; found: 310.2.

### Intermediate B. Methyl ((3R)-3-(6-amino-3-(methyl-d₃)-2-oxo-2,3-dihydro-1H-imidazo[4,5-c]pyridin-1-yl)cyclopentyl-1-d)carbamate

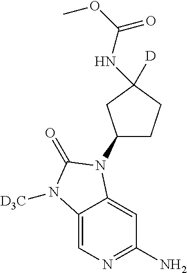 [SMILES: [2H]C1(NC(=O)OC)CC[C@@H](n2c(=O)n(C([2H])([2H])[2H])c3cnc(N)cc32)C1]

This compound was prepared according to the procedures described in Intermediate A, Step 1 to 9, with tert-butyl ((1R)-3-hydroxycyclopentyl-3-d)carbamate (Intermediate A, Step 1, mixture of diastereomers) replacing tert-butyl ((1R,3R)-3-hydroxycyclopentyl-3-d)carbamate in Step 2 to give the title compound as a mixture of two diastereomers. LCMS calculated for C₁₄H₁₆D₄N₅O₃(M+H)⁺: m/z=310.2; found: 310.2.

### Examples A-1-A-3

Examples A-1-A-3 of Table 6 were prepared according to the procedures described in U.S. Pat. No. 12,084,430, the disclosure of which is incorporated herein by reference in its entirety.

TABLE 6

| Ex. | | | LCMS | |
|---|---|---|---|---|
| No. | Name | Structure | [M + H]⁺ | ¹H NMR |
| A-1 | Methyl ((1R,3R)-3-(7-(3-fluoro- 1-(methyl-d₃)-1H- pyrazol-4-yl)-3- (methyl-d₃)-8-(1- (methyl-d₃)-1H- indazol-5-yl)-2-oxo-3,6- dihydroimidazo[4,5-d] pyrrolo[2,3-b]pyridin- 1(2H)-yl)cyclopentyl) carbamate | 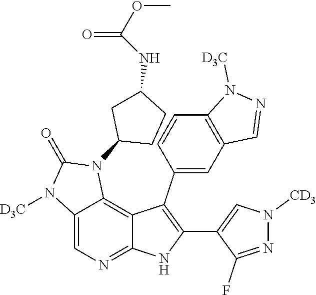 [SMILES: [2H]C([2H])([2H])n1cc(-c2[nH]c3ncc4c(c3c2-c2ccc3c(cnn3C([2H])([2H])[2H])c2)n([C@@H]2CC[C@@H](NC(=O)OC)C2)c(=O)n4C([2H])([2H])[2H])c(F)n1] | 567.3 | ¹H NMR δ ¹H NMR (600 MHz, DMSO) δ 11.87 (s, 1H), 8.12 (s, 1H), 8.03 (d, J = 11.5 Hz, 1H), 7.77- 7.65 (m, 2H), 7.46- 7.32 (m, 2H), 6.66- 6.23 (m, 1H), 3.89 (sext, J = 8.1 Hz, 1H), 3.82-3.52 (m, 1H), 3.52-3.41 (m, 3H), 2.14-1.46 (m, 3H), 1.45-0.81 (m, 2H), 0.38-−0.22 (m, 1H) |
| A-2 | Methyl ((1R,3R)-3-(7-(3-fluoro- 1-(methyl-d₃)-1H-pyrazol- 4-yl)-8-(4-fluorophenyl)- 3-(methyl-d₃)-2-oxo-3,6- dihydroimidazo[4,5-d] pyrrolo[2,3-b]pyridin- 1(2H)-yl)cyclopentyl) carbamate | 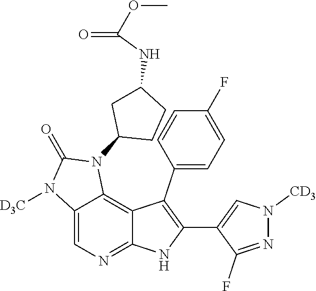 [SMILES: [2H]C([2H])([2H])n1cc(-c2[nH]c3ncc4c(c3c2-c2ccc(F)cc2)n([C@@H]2CC[C@@H](NC(=O)OC)C2)c(=O)n4C([2H])([2H])[2H])c(F)n1] | 528.2 | ¹H NMR (600 MHz, DMSO) δ 1H NMR (600 MHz, DMSO) δ 11.93 (s, 1H), 8.13 (d, J = 1.0 Hz, 1H), 7.44 (d, J = 1.9 Hz, 1H), 7.40 (s, 2H), 7.26 (t, J = 8.6 Hz, 2H), 6.91 (d, J = 6.7 Hz, 1H), 4.05 (sext, J = 7.8 Hz, 1H), 3.88 (s, 1H), 3.50 (s, 3H), 2.06 (ddd, J = 13.3, 8.4, 5.1 Hz, 1H), 1.96-1.80 (m, 2H), 1.46 (q, J = 9.0 Hz, 1H), 1.33-1.20 (m, 1H), 0.85-0.73 (m, 1H) |
| A-3 | Methyl ((1R,3R)-3-(8-(1,1- dimethyl-1,3- dihydroiso- benzofuran-5- yl)-7-(3-fluoro-1- (methyl-d3)- 1H-pyrazol- 4-yl)-3-(methyl- d3)-2-oxo-3,6- dihydroimidazo [4,5-d]pyrrolo [2,3-b]pyridin- 1(2H)-yl)cyclopentyl) carbamate | 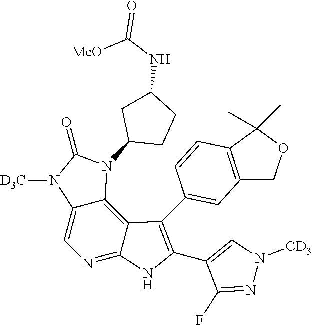 [SMILES: [2H]C([2H])([2H])n1cc(-c2[nH]c3ncc4c(c3c2-c2ccc3c(c2)COC3(C)C)n([C@@H]2CC[C@@H](NC(=O)OC)C2)c(=O)n4C([2H])([2H])[2H])c(F)n1] | 580.3 | ¹H NMR (600 MHz, DMSO) δ 11.91 (s, 1H), 8.12 (s, 1H), 7.45 (d, J = 24.5 Hz, 1H), 7.33-7.20 (m, 3H), 6.97 (s, 0.5H), 6.82 (s, 0.5H), 5.13 (d, J = 11.8 Hz, 0.5H), 5.00-4.89 (m, 1.5H), 4.06 (m, 1H), 3.91 (m, 1H), 3.49 (s, 3H), 2.05- 1.81 (m, 3H), 1.48 (d, J = 5.5 Hz, 6H), 1.38 (m, 1H), 1.21 (m, 1H), 0.66 (m, 1H) |

### Example B-1. Methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

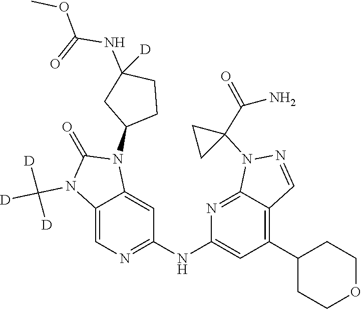 [SMILES: [2H]C1(NC(=O)OC)CC[C@@H](n2c(=O)n(C([2H])([2H])[2H])c3cnc(Nc4cc(C5CCOCC5)c5cnn(C6(C(N)=O)CC6)c5n4)cc32)C1]

### Step 1. tert-Butyl 2-(6-chloro-4-iodo-1H-pyrazolo[3,4-b]pyridin-1-yl)acetate

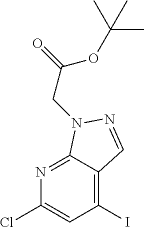 [SMILES: CC(C)(C)OC(=O)Cn1ncc2c(I)cc(Cl)nc21]

To a flask containing 6-chloro-4-iodo-1H-pyrazolo[3,4-b]pyridine (5.0 g, 17.9 mmol) and cesium carbonate (8.7 g, 26.8 mmol) in DMF (6.0 mL) was added tert-butyl 2-bromoacetate (2.9 mL, 19.7 mmol) After heating at 60° C. for 1 h, the reaction mixture was poured into an ice cold solution of saturated aqueous NH₄Cl and stirred for 10 min. The solid product from the reaction mixture was then filtered, followed by washing with water and hexanes to obtain the crude product. The crude material was purified by Biotage Isolera to afford the desired product. LCMS calculated for C₁₂H₁₄ClIN₃O₂ (M+H)⁺: m/z=394.0; found 394.0.

### Step 2. tert-Butyl 1-(6-chloro-4-iodo-1H-pyrazolo[3,4-b]pyridin-1-yl)cyclopropane-1-carboxylate

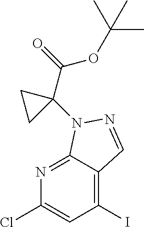 [SMILES: CC(C)(C)OC(=O)C1(n2ncc3c(I)cc(Cl)nc32)CC1]

To a flask containing tert-butyl 2-(6-chloro-4-iodo-1H-pyrazolo[3,4-b]pyridin-1-yl)acetate (4.5 g, 11.4 mmol) in THF (10.0 mL) at −78° C. was added LiHMDS (12.6 mL, 12.6 mmol, 1 M in THF) in a dropwise manner, After stirring at −78° C. for 10 min., a solution of 1,3,2-dioxathiolane 2,2-dioxide (1.56 g, 12.6 mmol) in THF (0.5 mL) was added to the reaction mixture. After 5 min, the reaction mixture was warmed up to 0° C. After stirring at 0° C. for 1 h, it was cooled down to −78° C., followed by the addition of another portion of LiHMDS (12.6 mL, 12.6 mmol, 1 M in THF) in a dropwise manner. The reaction mixture was allowed to warm up to rt by removing dry-ice bath. After stirring at r.t. for 16 h, the reaction was quenched with sat. aq. NH₄Cl (30 mL) and extraction with CH₂Cl₂. The organic layers were combined, dried over MgSO₄ and concentrated in vacuo. The crude material was purified by Biotage Isolera to afford the desired product. LCMS calculated for C₁₄H₁₆ClIN₃O₂ (M+H)⁺: m/z=420.0; found 420.0.

### Step 3. tert-Butyl 1-(6-chloro-4-(3,6-dihydro-2H-pyran-4-yl)-1H-pyrazolo[3,4-b]pyridin-1-yl)cyclopropane-1-carboxylate

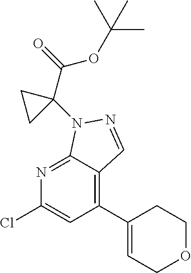 [SMILES: CC(C)(C)OC(=O)C1(n2ncc3c(C4=CCOCC4)cc(Cl)nc32)CC1]

To a vial containing tert-butyl 1-(6-chloro-4-iodo-1H-pyrazolo[3,4-b]pyridin-1-yl)cyclopropane-1-carboxylate (120 mg, 0.29 mmol), 2-(3,6-dihydro-2H-pyran-4-yl)-4,4,5,5-tetramethyl-1,3,2-dioxaborolane (66.1 mg, 0.32 mmol), tetrakis(triphenylphosphine)palladium(0) (33 mg, 0.029 mmol) and cesium carbonate (186 mg, 0.57 mmol) was added 1,4-dioxane (3.0 mL) and water (0.75 mL). The reaction mixture was flushed with nitrogen, then sealed and heated at 75° C. for 2 h. After cooling down to r.t., the reaction mixture was diluted with CH₂Cl₂, filtered through Celite and washed with CH₂Cl₂. The CH₂Cl₂ filtrates were combined, dried over MgSO₄ and concentrated in vacuo. The crude product was purified by Biotage Isolera to afford the desired compound. LCMS calculated for C₁₉H₂₃ClN₃₀₃ (M+H)⁺: m/z=376.1; found 376.1.

### Step 4. tert-Butyl 1-(4-(3,6-dihydro-2H-pyran-4-yl)-6-((1-((1R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclopropane-1-carboxylate

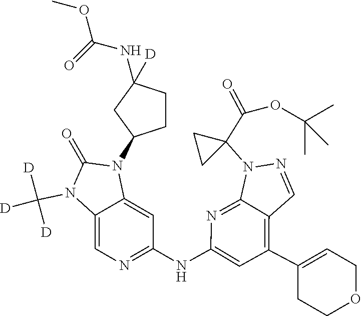 [SMILES: [2H]C1(NC(=O)OC)CC[C@@H](n2c(=O)n(C([2H])([2H])[2H])c3cnc(Nc4cc(C5=CCOCC5)c5cnn(C6(C(=O)OC(C)(C)C)CC6)c5n4)cc32)C1]

To a vial containing methyl ((3R)-3-(6-amino-3-(methyl-d₃)-2-oxo-2,3-dihydro-1H-imidazo[4,5-c]pyridin-1-yl)cyclopentyl-1-d)carbamate (80 mg, 0.26 mmol, Intermediate B), tert-butyl 1-(6-chloro-4-(3,6-dihydro-2H-pyran-4-yl)-1H-pyrazolo[3,4-b]pyridin-1-yl)cyclopropane-1-carboxylate (97 mg, 0.26 mmol), tris(dibenzylideneacetone)dipalladium(0) (24 mg, 0.026 mmol), 4,5-bis(diphenylphosphino)-9,9-dimethylxanthene (30 mg, 0.052 mmol) and cesium carbonate (253 mg, 0.78 mmol) was added 1,4-dioxane (5.0 mL). The reaction mixture was flushed with nitrogen, then sealed and heated at 105° C. for 16 h. After cooling down to r.t., the reaction mixture was diluted with CH₂Cl₂, filtered through Celite and washed with CH₂Cl₂. The CH₂Cl₂ filtrates were combined, dried over MgSO₄ and concentrated in vacuo. The crude product was purified by Biotage Isolera to afford the desired product. LCMS calculated for C₃₃H₃₇D₄N₈O₆ (M+H)⁺: m/z=649.3; found 649.2.

### Step 5. 1-(6-((1-((1R)-3-((Methoxycarbonyl)amino)cyclopentyl-3-d)-3-(methyl-d₃)-2-oxo-2,3-dihydro-1H-imidazo[4,5-c]pyridin-6-yl)amino)-4-(tetrahydro-2H-pyran-4-yl)-1H-pyrazolo[3,4-b]pyridin-1-yl)cyclopropane-1-carboxylic acid

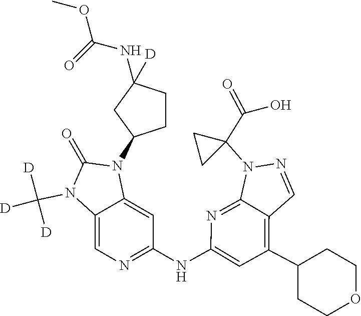 [SMILES: [2H]C1(NC(=O)OC)CC[C@@H](n2c(=O)n(C([2H])([2H])[2H])c3cnc(Nc4cc(C5CCOCC5)c5cnn(C6(C(=O)O)CC6)c5n4)cc32)C1]

To a vial containing tert-butyl 1-(4-(3,6-dihydro-2H-pyran-4-yl)-6-((1-((1R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclopropane-1-carboxylate (40 mg, 0.062 mmol) in MeOH (2.0 mL) was added Pd/C (6.6 mg, 10% w/w). The vial was purged with hydrogen gas for 5 min. and then stirred for 5 h under an atmosphere of hydrogen at 40° C. After cooling to room temperature, the reaction mixture was filtered through Celite and washed with CH₂Cl₂, followed by concentration of the filtrate in vacuo. The crude product was treated with 4 M HCl in 1,4-dioxane (2 mL). After heating at 60° C. for 30 min, the reaction mixture was concentrated in vacuo. The obtained crude product as the HCl salt of the title compound was used in the next step without further purification. LCMS calculated for C₂₉H₃₁D₄N₈O₆ (M+H)⁺: m/z=595.3; found 595.2.

### Step 6 Methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a vial containing 1-(6-((1-((1R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(tetrahydro-2H-pyran-4-yl)-1H-pyrazolo[3,4-b]pyridin-1-yl)cyclopropane-1-carboxylic acid (80 mg, 0.135 mmol), ammonia (1.0 mL, 0.5 M in dioxane) and DIPEA (0.12 mL, 0.68 mmol) in DMF (3 mL) was added HATU (77 mg, 0.20 mmol). After stirring at r.t. for 30 min, the crude product was diluted with acetonitrile and purified by prep-LCMS as the first peak of two diastereoisomers (XBridge C18 column, eluting with a gradient of 14.0→32.0% acetonitrile in water containing 0.10% TFA, at flow rate of 60 mL/min over 12 min). The desired product was collected at a retention time of 9.1 min as a single diastereomer. LCMS calculated for C₂₉H₃₂D₄N₉O₅ (M+H)⁺: m/z=594.3; found 594.2. ¹H NMR (500 MHz, DMSO) δ 10.96 (s, 1H), 8.32 (s, 1H), 8.27 (s, 1H), 7.80 (brs, 1H), 7.34 (s, 1H), 7.30 (s, 1H), 6.90 (s, 1H), 6.64 (s, 1H), 4.90-4.80 (m, 1H), 4.06-3.99 (m, 2H), 3.61-3.52 (m, 5H), 3.30-3.22 (m, 1H), 2.36 (dd, J=13.6, 7.6 Hz, 1H), 2.25-2.02 (m, 3H), 1.98-1.92 (m, 1H), 1.90-1.75 (m, 6H), 1.65 (dt, J=12.1, 7.9 Hz, 1H), 1.60-1.54 (m, 2H).

### Example B-2. Methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

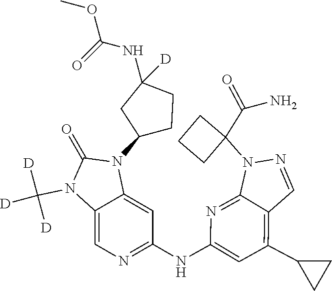 [SMILES: [2H]C1(NC(=O)OC)CC[C@@H](n2c(=O)n(C([2H])([2H])[2H])c3cnc(Nc4cc(C5CC5)c5cnn(C6(C(N)=O)CCC6)c5n4)cc32)C1]

### Step 1. Ethyl 1-(6-chloro-4-iodo-1H-pyrazolo[3,4-b]pyridin-1-yl)cyclobutane-1-carboxylate

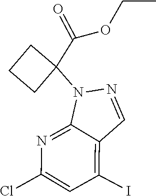 [SMILES: CCOC(=O)C1(n2ncc3c(I)cc(Cl)nc32)CCC1]

To a vial containing 2,6-dichloro-4-iodonicotinaldehyde (700 mg, 2.32 mmol) and ethyl 1-hydrazineylcyclobutane-1-carboxylate hydrochloride (470 mg, 2.43 mmol) in EtOH (3 mL) was added DIPEA (1.0 mL, 5.8 mmol). The reaction vial was sealed and heated 60° C. for 1 h. After cooling down to r.t., the reaction mixture was transferred to a microwave vial with the addition of DIPEA (1.0 mL, 5.8 mmol). The microwave vial was sealed and irradiated at 150° C. for 3 h. After cooling down to r.t., the reaction mixture was concentrated in vacuo. The obtained crude product was purified by Biotage Isolera to afford the desired product. LCMS calculated for C₁₃H₁₄ClIN₃O₂ (M+H)⁺: m/z=406.0; found 406.0.

### Step 2. Ethyl 1-(6-chloro-4-cyclopropyl-1H-pyrazolo[3,4-b]pyridin-1-yl)cyclobutane-1-carboxylate

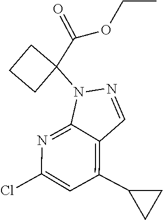 [SMILES: CCOC(=O)C1(n2ncc3c(C4CC4)cc(Cl)nc32)CCC1]

This compound was prepared according to the procedures described in Example B-1, Step 3, with ethyl 1-(6-chloro-4-iodo-1H-pyrazolo[3,4-b]pyridin-1-yl)cyclobutane-1-carboxylate replacing tert-butyl 1-(6-chloro-4-iodo-1H-pyrazolo[3,4-b]pyridin-1-yl)cyclopropane-1-carboxylate and cyclopropyltrifluoro-l4-borane, potassium salt replacing 2-(3,6-dihydro-2H-pyran-4-yl)-4,4,5,5-tetramethyl-1,3,2-dioxaborolane in Step 3. LCMS calculated for C₁₆H₁₉ClN₃O₂ (M+H)⁺: m/z=320.1; found 320.1.

### Step 3. Ethyl 1-(4-cyclopropyl-6-((1-((1R)-3-((methoxycarbonyl)amino)cyclopentyl-3-d)-3-(methyl-d)-2-oxo-2,3-dihydro-1H-imidazo[4,5-c]pyridin-6-yl)amino)-1H-pyrazolo[3,4-b]pyridin-1-yl)cyclobutane-1-carboxylate

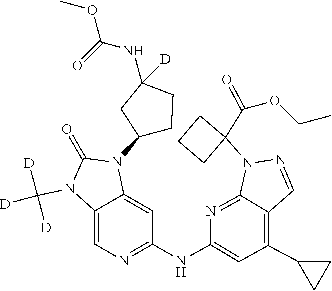 [SMILES: [2H]C1(NC(=O)OC)CC[C@@H](n2c(=O)n(C([2H])([2H])[2H])c3cnc(Nc4cc(C5CC5)c5cnn(C6(C(=O)OCC)CCC6)c5n4)cc32)C1]

This compound was prepared according to the procedures described in Example B-1, Step 4, with ethyl 1-(6-chloro-4-cyclopropyl-1H-pyrazolo[3,4-b]pyridin-1-yl)cyclobutane-1-carboxylate replacing tert-butyl 1-(6-chloro-4-(3,6-dihydro-2H-pyran-4-yl)-1H-pyrazolo[3,4-b]pyridin-1-yl)cyclopropane-1-carboxylate in Step 4. LCMS calculated for C₃₀H₃₃D₄N₈O₅ (M+H)⁺: m/z=593.3; found 593.3.

### Step 4. Methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a vial containing ethyl 1-(4-cyclopropyl-6-((1-((1R)-3-((methoxycarbonyl)amino)-cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (38 mg, 0.065 mmol) in THF/MeOH/water (1.5 mL, v/v/v=1/1/1) was added LiOH (16 mg, 0.67 mmol). After heating at 50° C. for 15 min, the reaction mixture was cooled to r.t. and diluted with CH₂Cl₂ (3 mL). The pH of the mixture was adjusted to ~4 with HCl aqueous solution, followed by the extraction with CH₂Cl₂ (3×5 mL). The organic layers were combined, dried over MgSO₄ and concentrated in vacuo. The obtained crude product was dissolved in DMF (1 mL), followed by the addition of ammonia (0.5 mL, 0.5 M in dioxane), DIPEA (0.12 mL, 0.68 mmol) and HATU (39 mg, 0.10 mmol). After stirring at r.t. for 30 min, the crude product was diluted with acetonitrile and purified by prep-LCMS as the first peak of two diastereoisomers (XBridge C18 column, eluting with a gradient of 17.8→35.8% acetonitrile in water containing 0.1% TFA, at flow rate of 60 mL/min over 12 min). The desired product was collected at a retention time of 10.8 min as a single diastereomer. LCMS calculated for C₂₈H₃₀D₄N₉O₄ (M+H)⁺: m/z=564.3; found 564.3.

### Example B-3. Methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

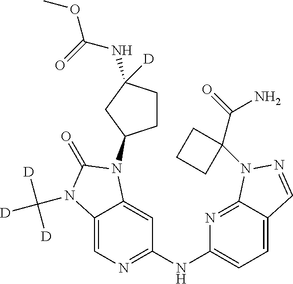 [SMILES: [2H]C([2H])([2H])n1c(=O)n([C@@H]2CC[C@@]([2H])(NC(=O)OC)C2)c2cc(Nc3ccc4cnn(C5(C(N)=O)CCC5)c4n3)ncc21]

### Step 1. Ethyl 1-(6-bromo-1H-pyrazolo[3,4-b]pyridin-1-yl)cyclobutane-1-carboxylate

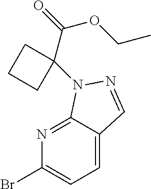 [SMILES: CCOC(=O)C1(n2ncc3ccc(Br)nc32)CCC1]

To a microwave vial containing 6-bromo-2-fluoronicotinaldehyde (2.0 g, 9.8 mmol) was added ethyl 1-hydrazineylcyclobutane-1-carboxylate hydrochloride (2.0 g, 10.3 mmol), NMP (9.8 mL) and DIPEA (8.6 mL, 49.0 mmol), then it was sealed and irradiated in the microwave at 180° C. for 2 h. After cooling down to r.t., the reaction mixture was diluted with EtOAc (50 mL) and water (20 mL). After extraction with EtOAc for 3 times, the organic layers were combined, washed with brine, dried over MgSO₄ and concentrated in vacuo. The crude material was purified by Biotage Isolera to afford the desired product. LCMS calculated for C₁₃H₁₅BrN₃O₂ (M+H)⁺: m/z=324.0/326.0; found 324.0/326.0.

### Step 2. 1-(6-Bromo-1H-pyrazolo[3,4-b]pyridin-1-yl)cyclobutane-1-carboxylic Acid

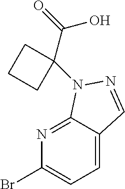 [SMILES: O=C(O)C1(n2ncc3ccc(Br)nc32)CCC1]

To a vial containing ethyl 1-(6-bromo-1H-pyrazolo[3,4-b]pyridin-1-yl)cyclobutane-1-carboxylate (2.0 g, 6.2 mmol) in THF (5 mL) was added lithium hydroxide, 2.0 M aq. (5 mL, 10.00 mmol). After heating at 60° C. for 16 h, the reaction mixture was cooled to r.t. and concentrated in vacuo to remove THF. The pH of the mixture was adjusted to ~1 using 1 M HCl aqueous solution until a precipitate formed. The reaction mixture was filtered. The filter cake was washed with water and hexanes, followed by air drying. The obtained crude product was used in the next step without further purification. LCMS calculated for C₁₁H₁₁BrN₃O₂ (M+H)⁺: m/z=296.0/298.0; found 296.0/298.0.

### Step 3. 1-(6-Bromo-1H-pyrazolo[3,4-b]pyridin-1-yl)cyclobutane-1-carboxamide

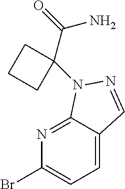 [SMILES: NC(=O)C1(n2ncc3ccc(Br)nc32)CCC1]

To a vial containing 1-(6-bromo-1H-pyrazolo[3,4-b]pyridin-1-yl)cyclobutane-1-carboxylic acid (3.41 g, 11.5 mmol) in DMF (10.0 mL) at 0° C. was added CDI (2.80 g, 17.3 mmol) as a suspension in 5 mL DMF. After heating at 60° C. for 1 h, the reaction mixture was cooled to r.t., followed by the addition of ammonium hydroxide (4.5 mL, 115 mmol). After heating at 60° C. for 2 h, the reaction mixture was diluted with CHCl₃/IPA (v/v=3/1) and 1 M HCl, followed by extraction with CHCl₃/IPA (v/v=3/1) 3 times. The organic phase was washed with brine, dried over MgSO₄ and concentrated in vacuo The crude material was purified by Biotage Isolera to afford the desired product. LCMS calculated for C₁₁H₁₂BrN₄O (M+H)⁺: m/z=295.0/297.0; found 295.0/297.0.

### Step 4. Methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (21 mg, 68 μmol, Intermediate A), 1-(6-bromo-1H-pyrazolo[3,4-b]pyridin-1-yl)cyclobutane-1-carboxamide (19 mg, 65 μmol), tris(dibenzylideneacetone)-dipalladium(0) (6 mg, 6.5 μmol), 4,5-bis(diphenylphosphino)-9,9-dimethylxanthene (7.5 mg, 13 μmol) and cesium carbonate (64 mg, 19.4 μmol) was evacuated and backfilled with nitrogen three times, followed by the addition of 1,4-dioxane (1.0 mL). The vial was sealed and heated to 120° C. for 2 h. After cooling to r.t., the reaction mixture was diluted with CH₂Cl₂, filtered through Celite and washed with CH₂Cl₂. The CH₂Cl₂ filtrates were combined, dried over MgSO₄ and concentrated in vacuo. The crude product was diluted with acetonitrile and purified by prep-LCMS (XBridge C18 column, eluting with a gradient of acetonitrile/water containing 0.1% TFA, at flow rate of 60 mL/min). LCMS calculated for C₂₅H₂₆D₄N₉O₄ (M+H)⁺: m/z=524.3; found 524.3. ¹H NMR (500 MHz, DMSO) δ 11.54 (s, 1H), 8.28 (s, 1H), 8.25 (d, J=8.7 Hz, 1H), 8.18 (s, 1H), 7.50-7.42 (m, 2H), 7.38 (s, 1H), 7.32 (s, 1H), 6.99 (d, J=8.7 Hz, 1H), 5.04 (p, J=8.5 Hz, 1H), 3.55 (s, 3H), 3.00-2.81 (m, 4H), 2.34 (dd, J=13.6, 7.8 Hz, 1H), 2.26-2.12 (m, 2H), 2.11-2.03 (m, 1H), 2.02-1.93 (m, 3H), 1.66 (dt, J=12.2, 7.9 Hz, 1H).

### Example B-4. Methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate and Methyl ((1S,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

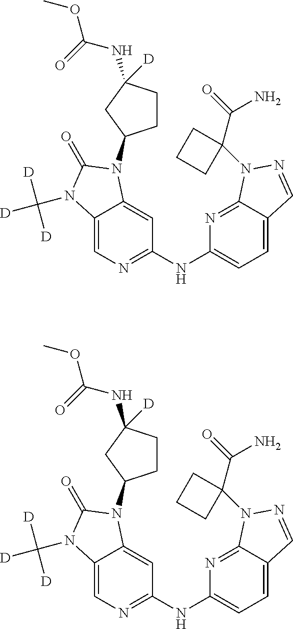 [SMILES: [2H]C([2H])([2H])n1c(=O)n([C@@H]2CC[C@@]([2H])(NC(=O)OC)C2)c2cc(Nc3ccc4cnn(C5(C(N)=O)CCC5)c4n3)ncc21 ; [2H]C([2H])([2H])n1c(=O)n([C@@H]2CC[C@]([2H])(NC(=O)OC)C2)c2cc(Nc3ccc4cnn(C5(C(N)=O)CCC5)c4n3)ncc21]

This compound was prepared according to the procedures described in Example B-3, Step 4, with methyl ((3R)-3-(6-amino-3-(methyl-d₃)-2-oxo-2,3-dihydro-1H-imidazo[4,5-c]pyridin-1-yl)cyclopentyl-1-d)carbamate (Intermediate B) replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate (Intermediate A) in Step 4. The crude product was purified by prep-LCMS as the second peak of two diastereoisomers (XBridge C18 column, eluting with a gradient of 14.0→32.0% acetonitrile in water containing 0.1% TFA, at flow rate of 60 mL/min over 12 min). Retention time was 10.1 min for peak 1 and 10.7 min for peak 2. The desired product was collected at a retention time of 10.7 min as a single diastereomer. LCMS calculated for C₂₅H₂₆D₄N₉O₄ (M+H)⁺: m/z=524.3; found 524.3. ¹H NMR (600 MHz, DMSO) δ 11.47 (s, 1H), 8.28 (s, 1H), 8.23 (d, J=8.6 Hz, 1H), 8.16 (s, 1H), 7.63 (brs, 1H), 7.41 (s, 1H), 7.39 (s, 1H), 7.18 (brs, 1H), 6.99 (d, J=8.7 Hz, 1H), 4.93 (p, J=8.7 Hz, 1H), 3.55 (s, 3H), 2.99-2.84 (m, 4H), 2.27 (dd, J=12.5, 8.2 Hz, 1H), 2.23-2.15 (m, 1H), 2.13-2.04 (m, 2H), 2.03-1.92 (m, 3H), 1.86-1.81 (m, 1H).

### Example B-5. Methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

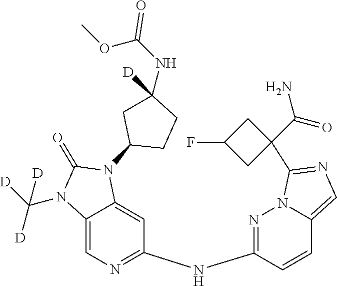 [SMILES: [2H]C([2H])([2H])n1c(=O)n([C@@H]2CC[C@@]([2H])(NC(=O)OC)C2)c2cc(Nc3ccc4cnc(C5(C(N)=O)CC(F)C5)n4n3)ncc21]

### Step 1. Isopropyl 1-(((6-chloropyridazin-3-yl)methyl)carbamoyl)-3-fluorocyclobutane-1-carboxylate

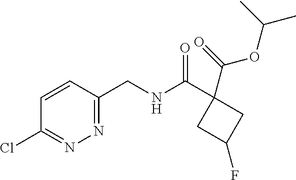 [SMILES: CC(C)OC(=O)C1(C(=O)NCc2ccc(Cl)nn2)CC(F)C1]

To a flask containing 3-fluoro-1-(isopropoxycarbonyl)cyclobutane-1-carboxylic acid (9.0 g, 43.9 mmol) in DCM (84 mL) was added N-ethyl-N-isopropylpropan-2-amine (22.3 mL, 125 mmol) followed by HATU (23.8 g, 62.7 mmol) and (6-chloropyridazin-3-yl)methanamine hydrochloride (7.6 g, 41.8 mmol). After stirring at r.t for 1 h, water (80 mL) was then added, and the mixture was extracted with DCM (3×40 mL). The organic phases were combined, dried over Na₂SO₄, filtered, concentrated in vacuo and the residue was purified by Biotage Isolera. LCMS calculated for C₁₄H₁₈ClFN₃O₃ (M+H)⁺: m/z=330.1; found 330.1.

### Step 2. Isopropyl 1-(2-chloroimidazo[1,5-b]pyridazin-7-yl)-3-fluorocyclobutane-1-carboxylate

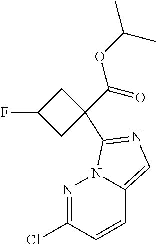 [SMILES: CC(C)OC(=O)C1(c2ncc3ccc(Cl)nn23)CC(F)C1]

A round-bottom flask containing a solution of isopropyl 1-(((6-chloropyridazin-3-yl)methyl)carbamoyl)-3-fluorocyclobutane-1-carboxylate (9.5 g, 28.8 mmol) in acetonitrile (450 mL) and phosphoryl trichloride (44.2 g, 288 mmol) was refluxed at 115° C. for 16 h. The reaction mixture was concentrated in vacuo and the residue was basified with sat. NaHCO₃ and extracted with EtOAc (3×50 mL). The organic phases were combined, dried over Na₂SO₄ and concentrated in vacuo. The obtained crude product was purified by Biotage Isolera, which was then subjected to SFC chiral purification to separate two diastereomers (ChiralTech IK 5 μm 30×250 mm column, eluting with a gradient of 15% IPA in CO₂, at flow rate of 90 mL/min). Retention time was 3.5 min for peak 1 and 4.9 min for peak 2. The titled product (peak 2) was obtained as a single diastereomer. LCMS calculated for C₁₄H₁₆ClFN₃O₂ (M+H)⁺: m/z=312.1; found 312.1.

### Step 3. Isopropyl 3-fluoro-1-(2-((1-((1R,3R)-3-((methoxycarbonyl)amino)cyclopentyl-3-d)-3-(methyl-d)-2-oxo-2,3-dihydro-1H-imidazo[4,5-c]pyridin-6-yl)amino)imidazo[1,5-b]pyridazin-7-yl)cyclobutane-1-carboxylate

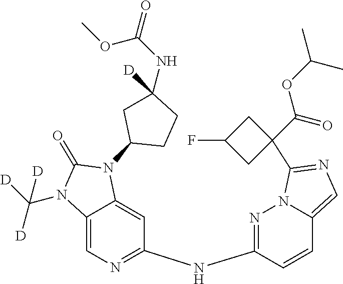 [SMILES: [2H]C([2H])([2H])n1c(=O)n([C@@H]2CC[C@@]([2H])(NC(=O)OC)C2)c2cc(Nc3ccc4cnc(C5(C(=O)OC(C)C)CC(F)C5)n4n3)ncc21]

A reaction vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (77 mg, 0.24 mmol), isopropyl 1-(2-chloroimidazo[1,5-b]pyridazin-7-yl)-3-fluorocyclobutane-1-carboxylate (Example B-5-Step 2-Peak 2) (75 mg, 0.24 mmol), cesium carbonate (234 mg, 0.7 mmol), Pd₂(dba)₃ (44 mg, 0.05 mmol), and xantphos (55 mg, 0.1 mmol) was evacuated and backfilled with nitrogen. 1,4-Dioxane (2.5 mL) was added to the reaction mixture, which was then stirred at 110° C. for 1 h. The reaction mixture was cooled to r.t and diluted with EtOAc. The organic layer was subsequently washed with water and brine, dried over Na₂SO₄, filtered, and the solvents were evaporated in vacuo. The crude material was purified by Biotage Isolera. LCMS calculated for C₂₈H₃₀D₄FN₈O₅ (M+H)⁺: m/z=585.3; found 585.3.

### Step 4. 3-Fluoro-1-(2-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)imidazo[1,5-b]pyridazin-7-yl)cyclobutane-1-carboxylic Acid

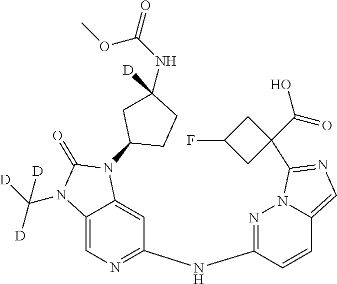 [SMILES: [2H]C([2H])([2H])n1c(=O)n([C@@H]2CC[C@@]([2H])(NC(=O)OC)C2)c2cc(Nc3ccc4cnc(C5(C(=O)O)CC(F)C5)n4n3)ncc21]

A solution of isopropyl 3-fluoro-1-(2-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)imidazo[1,5-b]pyridazin-7-yl)cyclobutane-1-carboxylate (122 mg, 0.21 mmol) and lithium hydroxide (49 mg, 2.1 mmol) in MeOH (1.5 mL), THF (1.5 mL), water (1.5 mL) was stirred at 65° C. for 5 h. The reaction was cooled to ambient temperature, acidified to pH 2-3 using 12 M HCl and diluted with EtOAc (6 mL). The layers were separated, and the aqueous layer was further extracted with EtOAc (3×6 mL). The organic layer was dried over Na₂SO₄, filtered, and concentrated in vacuo. The crude residue was carried forward without any further purification. LCMS calculated for C₂₅H₂₄D₄FN₈O₅ (M+H)⁺: m/z=543.2; found 543.2.

### Step 5. Methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

To a vial containing 3-fluoro-1-(2-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)imidazo[1,5-b]pyridazin-7-yl)cyclobutane-1-carboxylic acid (108 mg, 0.2 mmol), HATU (155 mg, 0.4 mmol) in DMF (2 mL) was added N-ethyl-N-isopropylpropan-2-amine (0.1 mL, 0.6 mmol) followed by 0.4 M ammonia in dioxane (1.5 mL, 0.6 mmol). The reaction mixture was stirred at r.t for 1 h. The reaction was quenched with water (10 mL) and extracted with EtOAc (3×10 mL). The combined organic layers were dried over Na₂SO₄, filtered, and concentrated in vacuo. The crude material was diluted with acetonitrile and purified by prep-LCMS (XBridge C18 column, eluting with a gradient of acetonitrile/water containing 0.1% TFA, at flow rate of 60 mL/min). The product was obtained as a single diastereomer. LCMS calculated for C₂₅H₂₅D₄FN₉O₄ (M+H)⁺: m/z=542.3; found 542.3. ¹H NMR (400 MHz, DMSO) δ 10.92 (s, 1H), 8.26 (s, 1H), 8.15 (d, J=9.7 Hz, 1H), 7.85 (s, 1H), 7.56 (s, 1H), 7.48 (s, 1H), 7.39 (s, 1H), 7.29 (s, 1H), 6.91 (d, J=9.7 Hz, 1H), 5.43-5.21 (m, 1H), 5.14 (p, J=8.2 Hz, 1H), 3.35-3.23 (m, 2H), 3.02-2.80 (m, 2H), 2.51 (s, 3H), 2.37 (dd, J=13.5, 7.0 Hz, 1H), 2.23-2.14 (m, 2H), 2.11-2.04 (m, 1H), 1.99 (dd, J=13.5, 9.7 Hz, 1H), 1.81-1.54 (m, 1H).

### Example B-6. Methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

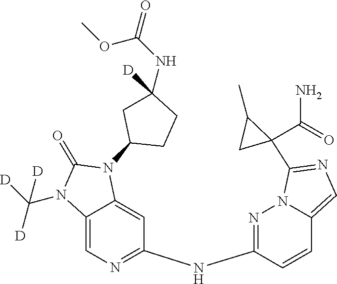 [SMILES: [2H]C([2H])([2H])n1c(=O)n([C@@H]2CC[C@@]([2H])(NC(=O)OC)C2)c2cc(Nc3ccc4cnc(C5(C(N)=O)CC5C)n4n3)ncc21]

### Step 1. Methyl 1-(2-chloroimidazo[1,5-b]pyridazin-7-yl)-2-methylcyclopropane-1-carboxylate

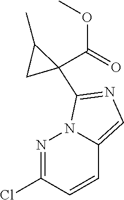 [SMILES: COC(=O)C1(c2ncc3ccc(Cl)nn23)CC1C]

The title compound was prepared according to the procedures described in Example B-5, Step 1-2, with 1-(methoxycarbonyl)-2-methylcyclopropane-1-carboxylic acid replacing 3-fluoro-1-(isopropoxycarbonyl)cyclobutane-1-carboxylic acid in Step 1. The obtained crude product was purified by Biotage Isolera, which was then subjected to SFC chiral purification to separate two diastereomers (ChiralTech IK 5 μm 21×250 mm column, eluting with a gradient of 8% MeOH in CO₂, at flow rate of 85 mL/min). Retention time was 7.6 min for peak 1 and 9.2 min for peak 2. The titled compound (peak 1) was obtained as a single diastereomer. LCMS calculated for C₁₂H₁₃ClN₃O₂ (M+H)⁺: m/z=266.1; found: 266.1.

### Step 2. Methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example B-5, Step 3-5, with methyl 1-(2-chloroimidazo[1,5-b]pyridazin-7-yl)-2-methylcyclopropane-1-carboxylate (peak 1) replacing isopropyl 1-(2-chloroimidazo[1,5-b]pyridazin-7-yl)-3-fluorocyclobutane-1-carboxylate in Step 3. The desired product was obtained as a single diastereomer. LCMS calculated for C₂₅H₂₆D₄N₉O₄ (M+H)⁺: m/z=524.3; found: 524.3.

### Example B-7. Methyl ((1R,3R)-3-(6-((7-(1-carbamoyl-3-fluorocyclobutyl)-5-cyclopropylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

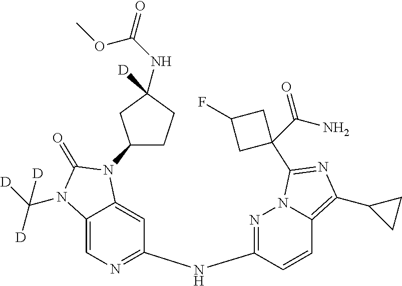 [SMILES: [2H]C([2H])([2H])n1c(=O)n([C@@H]2CC[C@@]([2H])(NC(=O)OC)C2)c2cc(Nc3ccc4c(C5CC5)nc(C5(C(N)=O)CC(F)C5)n4n3)ncc21]

### Step 1. Isopropyl 1-(2-chloro-5-iodoimidazo[1,5-b]pyridazin-7-yl)-3-fluorocyclobutane-1-carboxylate

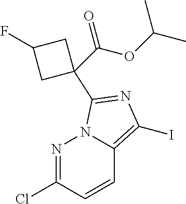 [SMILES: CC(C)OC(=O)C1(c2nc(I)c3ccc(Cl)nn23)CC(F)C1]

To a 40 mL vial containing isopropyl 1-(2-chloroimidazo[1,5-b]pyridazin-7-yl)-3-fluorocyclobutane-1-carboxylate (Example B-5-Step 2-Peak 2) (0.62 g, 2 mmol) in DCM (5 mL) and DMF (5 mL) was added 1-iodopyrrolidine-2,5-dione (0.9 g, 4 mmol) at r.t. After stirring at 80° C. for 1 h, the reaction mixture was cooled down, quenched with water, and extracted with EtOAc. The organic layers were combined, dried over Na₂SO₄, filtered, and the solvents were evaporated in vacuo. The crude material was purified by Biotage Isolera. LCMS calculated for C₁₄H₁₅ClFIN₃O₂ (M+H)⁺: m/z=438.1; found 438.1.

### Step 2. Isopropyl 1-(2-((2,4-dimethoxybenzyl)amino)-5-iodoimidazo[1,5-b]pyridazin-7-yl)-3-fluorocyclobutane-1-carboxylate

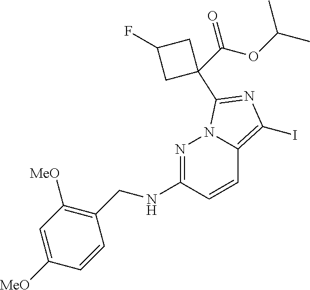 [SMILES: COc1ccc(CNc2ccc3c(I)nc(C4(C(=O)OC(C)C)CC(F)C4)n3n2)c(OC)c1]

To a vial containing isopropyl 1-(2-chloro-5-iodoimidazo[1,5-b]pyridazin-7-yl)-3-fluorocyclobutane-1-carboxylate (190 mg, 0.43 mmol) and (2,4-dimethoxyphenyl)methanamine (132 μL, 0.87 mmol) in DMSO (2 mL) was added N-ethyl-N-isopropylpropan-2-amine (232 μL, 1.3 mmol), which was then stirred at 110° C. for 2 h. The reaction mixture was cooled to r.t and water was added. The solid was then filtered and washed with water. The crude material was used in the next step without further purification. LCMS calculated for C₂₃H₂₇FIN₄O₄ (M+H)⁺: m/z=569.1; found 569.1.

### Step 3. Isopropyl 1-(2-amino-5-cyclopropylimidazo[1,5-b]pyridazin-7-yl)-3-fluorocyclobutane-1-carboxylate

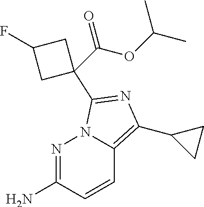 [SMILES: CC(C)OC(=O)C1(c2nc(C3CC3)c3ccc(N)nn23)CC(F)C1]

To a vial containing isopropyl 1-(2-((2,4-dimethoxybenzyl)amino)-5-iodoimidazo[1,5-b]pyridazin-7-yl)-3-fluorocyclobutane-1-carboxylate (120 mg, 0.21 mmol), Pd₂(dba)₃ (19 mg, 0.021 mmol), Qphos (30 mg, 0.042 mmol) in THF (3 mL) was added 0.5 M cyclopropylzinc(II) bromide in THF (1.6 mL, 0.63 mmol) under N2 atmosphere. The reaction was stirred at 75° C. for 30 min. The solution was cooled down, diluted with water, and extracted with EtOAc. The organic layer was dried over Na₂SO₄, filtered, and the solvents were evaporated in vacuo. The crude material was then dissolved in TFA (4 mL) and stirred at 80° C. for 5 min. The reaction mixture was cooled to r.t, concentrated to remove volatiles, diluted with EtOAc and neutralized with sat. NaHCO₃. The organic layer was subsequently washed with water and brine, dried over Na₂SO₄, filtered, and the solvents were evaporated in vacuo. The crude material was purified by Biotage Isolera. LCMS calculated for C₁₇H₂₂FN₄O₂ (M+H)⁺: m/z=333.2; found 333.2.

### Step 4. Methyl ((1R,3R)-3-(6-((7-(1-carbamoyl-3-fluorocyclobutyl)-5-cyclopropylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example B-5, Step 3-5, with isopropyl 1-(2-amino-5-cyclopropylimidazo[1,5-b]pyridazin-7-yl)-3-fluorocyclobutane-1-carboxylate replacing isopropyl 1-(2-chloroimidazo[1,5-b]pyridazin-7-yl)-3-fluorocyclobutane-1-carboxylate, and methyl ((1R,3R)-3-(6-chloro-3-(methyl-d₃)-2-oxo-2,3-dihydro-1H-imidazo[4,5-c]pyridin-1-yl)cyclopentyl-1-d)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 3. The desired product was obtained as a single diastereomer. LCMS calculated for C₂₈H₂₉D₄FN₉O₄ (M+H)⁺: m/z=582.3; found 582.3.

### Example B-8. Methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d)-2-oxo-2,3-dihydro-1H-imidazo[4,5-c]pyridin-1-yl)cyclopentyl-1-d)carbamate

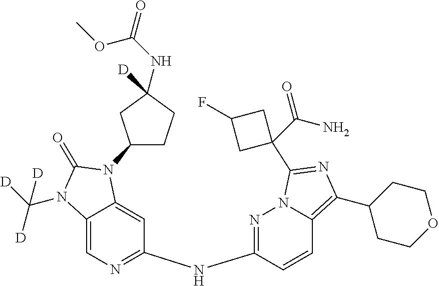 [SMILES: [2H]C([2H])([2H])n1c(=O)n([C@@H]2CC[C@@]([2H])(NC(=O)OC)C2)c2cc(Nc3ccc4c(C5CCOCC5)nc(C5(C(N)=O)CC(F)C5)n4n3)ncc21]

### Step 1. Isopropyl 1-(2-amino-5-(tetrahydro-2H-pyran-4-yl)imidazo[1,5-b]pyridazin-7-yl)-3-fluorocyclobutane-1-carboxylate

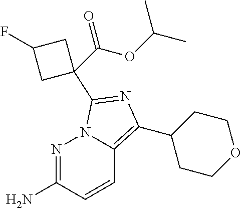 [SMILES: CC(C)OC(=O)C1(c2nc(C3CCOCC3)c3ccc(N)nn23)CC(F)C1]

The title compound was prepared according to the procedures described in Example B-7, Step 3, with (tetrahydro-2H-pyran-4-yl)zinc(II) bromide replacing cyclopropylzinc(II) bromide. LCMS calculated for C₁₉H₂₆FN₄O₃ (M+H)⁺: m/z=377.2; found 377.2.

### Step 2. Methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example B-5, Step 3-5, with isopropyl 1-(2-amino-5-(tetrahydro-2H-pyran-4-yl)imidazo[1,5-b]pyridazin-7-yl)-3-fluorocyclobutane-1-carboxylate replacing isopropyl 1-(2-chloroimidazo[1,5-b]pyridazin-7-yl)-3-fluorocyclobutane-1-carboxylate, and methyl ((1R,3R)-3-(6-chloro-3-(methyl-d₃)-2-oxo-2,3-dihydro-1H-imidazo[4,5-c]pyridin-1-yl)cyclopentyl-1-d)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 3. The desired product was obtained as a single diastereomer. LCMS calculated for C₃₀H₃₃D₄FN₉O₅ (M+H)⁺: m/z=626.3; found 626.3.

### Example B-9. Methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

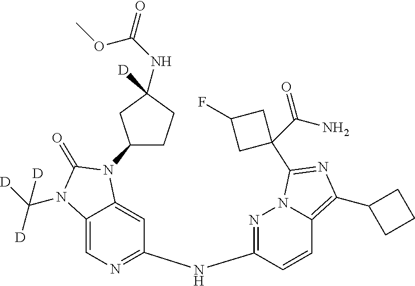 [SMILES: [2H]C([2H])([2H])n1c(=O)n([C@@H]2CC[C@@]([2H])(NC(=O)OC)C2)c2cc(Nc3ccc4c(C5CCC5)nc(C5(C(N)=O)CC(F)C5)n4n3)ncc21]

### Step 1. Isopropyl 1-(2-amino-5-cyclobutylimidazo[1,5-b]pyridazin-7-yl)-3-fluorocyclobutane-1-carboxylate

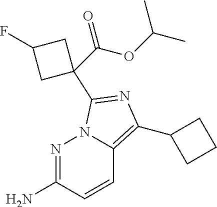 [SMILES: CC(C)OC(=O)C1(c2nc(C3CCC3)c3ccc(N)nn23)CC(F)C1]

The title compound was prepared according to the procedures described in Example B-7, Step 3, with cyclobutylzinc(II) bromide replacing cyclopropylzinc(II) bromide. LCMS calculated for C₁₈H₂₄FN₄O₂ (M+H)⁺: m/z=347.2; found 347.2.

### Step 2. Methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example B-5, Step 3-5, with isopropyl 1-(2-amino-5-cyclobutylimidazo[1,5-b]pyridazin-7-yl)-3-fluorocyclobutane-1-carboxylate replacing isopropyl 1-(2-chloroimidazo[1,5-b]pyridazin-7-yl)-3-fluorocyclobutane-1-carboxylate, and methyl ((1R,3R)-3-(6-chloro-3-(methyl-d₃)-2-oxo-2,3-dihydro-1H-imidazo[4,5-c]pyridin-1-yl)cyclopentyl-1-d)carbamate replacing methyl ((1R,3R)-3-(6-amino-3-(methyl-d₃)-2-oxo-2,3-dihydro-1H-imidazo[4,5-c]pyridin-1-yl)cyclopentyl-1-d)carbamate in Step 3. The desired product was obtained as a single diastereomer. LCMS calculated for C₂₉H₃₁D₄FN₉O₄ (M+H)⁺: m/z=596.3; found 596.3.

### Example B-10. Methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate

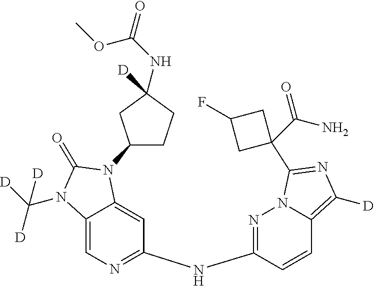 [SMILES: [2H]c1nc(C2(C(N)=O)CC(F)C2)n2nc(Nc3cc4c(cn3)n(C([2H])([2H])[2H])c(=O)n4[C@@H]3CC[C@@]([2H])(NC(=O)OC)C3)ccc12]

### Step 1. Isopropyl 1-(2-aminoimidazo[1,5-b]pyridazin-7-yl-5-d)-3-fluorocyclobutane-1-carboxylate

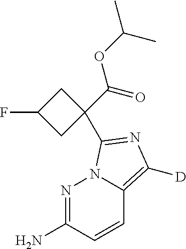 [SMILES: [2H]c1nc(C2(C(=O)OC(C)C)CC(F)C2)n2nc(N)ccc12]

A suspension of isopropyl 1-(2-((2,4-dimethoxybenzyl)amino)-5-iodoimidazo[1,5-b]pyridazin-7-yl)-3-fluorocyclobutane-1-carboxylate (Example B-7-Step 2) (50 mg, 0.09 mmol) and Pd on carbon (10%, 11 mg, 0.009 mmol) in MeOH (5 ml) was stirred at 60° C. under a balloon of D₂ overnight. The reaction mixture was then filtered and concentrated in vacuo. The crude material was then dissolved in TFA (2 mL) and stirred at 60° C. for 5 min. The reaction mixture was cooled to r.t, concentrated to remove volatiles, diluted with EtOAc and neutralized with sat. NaHCO₃. The organic layer was subsequently washed with water and brine, dried over Na₂SO₄, filtered, and the solvents were evaporated in vacuo. The crude material was purified by Biotage Isolera. LCMS calculated for C₁₄H₁₇DFN₄O₂ (M+H)⁺: m/z=294.2; found 294.2.

### Step 2. Methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate

The title compound was prepared according to the procedures described in Example B-5, Step 3-5, with isopropyl 1-(2-aminoimidazo[1,5-b]pyridazin-7-yl-5-d)-3-fluorocyclobutane-1-carboxylate replacing isopropyl 1-(2-chloroimidazo[1,5-b]pyridazin-7-yl)-3-fluorocyclobutane-1-carboxylate, and 6-chloro-1-isopropyl-3-(methyl-d₃)-1,3-dihydro-2H-imidazo[4,5-c]pyridin-2-one replacing 6-amino-1-isopropyl-3-(methyl-d₃)-1,3-dihydro-2H-imidazo[4,5-c]pyridin-2-one in Step 3. The desired product was obtained as a single diastereomer. LCMS calculated for C₂₅H₂₄D₅FN₉O₄ (M+H)⁺: m/z=543.3; found: 543.3.

### Example B-11. Methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

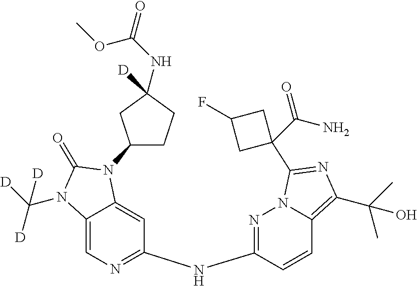 [SMILES: [2H]C([2H])([2H])n1c(=O)n([C@@H]2CC[C@@]([2H])(NC(=O)OC)C2)c2cc(Nc3ccc4c(C(C)(C)O)nc(C5(C(N)=O)CC(F)C5)n4n3)ncc21]

### Step 1. Isopropyl 1-(2-chloro-5-formylimidazo[1,5-b]pyridazin-7-yl)-3-fluorocyclobutane-1-carboxylate

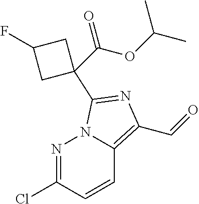 [SMILES: CC(C)OC(=O)C1(c2nc(C=O)c3ccc(Cl)nn23)CC(F)C1]

To a vial containing DMF (2 mL) was added dropwise phosphoryl trichloride (1.5 g, 10 mmol) at r.t. The mixture was stirred at r.t for 15 min. To this solution was added isopropyl 1-(2-chloroimidazo[1,5-b]pyridazin-7-yl)-3-fluorocyclobutane-1-carboxylate (Example B-5-Step 2-Peak 2) (0.31 g, 1 mmol) and the mixture was stirred at r.t for 5 min before heating to 80° C. for 2 h. The reaction was allowed to cool down, poured into ice water, neutralized with sat. NaHCO₃ solution and extracted with ethyl acetate (3×10 mL). The combined organic layers were subsequently washed with brine, dried over Na₂SO₄, filtered, and the solvents were evaporated in vacuo. The crude material was purified by Biotage Isolera. LCMS calculated for C₁₅H₁₆ClFN₃O₃ (M+H)⁺: m/z=340.1; found 340.1.

### Step 2. Methyl ((1R,3R)-3-(6-((7-(1-carbamoyl-3-fluorocyclobutyl)-5-formylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

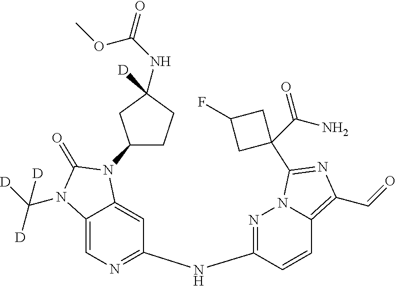 [SMILES: [2H]C([2H])([2H])n1c(=O)n([C@@H]2CC[C@@]([2H])(NC(=O)OC)C2)c2cc(Nc3ccc4c(C=O)nc(C5(C(N)=O)CC(F)C5)n4n3)ncc21]

The title compound was prepared according to the procedures described in Example B-5, Step 3-5, with isopropyl 1-(2-chloro-5-formylimidazo[1,5-b]pyridazin-7-yl)-3-fluorocyclobutane-1-carboxylate replacing isopropyl 1-(2-chloroimidazo[1,5-b]pyridazin-7-yl)-3-fluorocyclobutane-1-carboxylate in Step 3. The crude material was purified by Biotage Isolera. LCMS calculated for C₂₆H₂₅D₄FN₉O₅ (M+H)⁺: m/z=570.3; found: 570.3.

### Step 3. Methyl ((1R,3R)-3-(6-((7-(1-carbamoyl-3-fluorocyclobutyl)-5-(1-hydroxyethyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

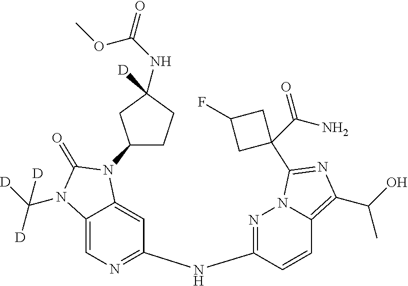 [SMILES: [2H]C([2H])([2H])n1c(=O)n([C@@H]2CC[C@@]([2H])(NC(=O)OC)C2)c2cc(Nc3ccc4c(C(C)O)nc(C5(C(N)=O)CC(F)C5)n4n3)ncc21]

To a vial containing methyl ((1R,3R)-3-(6-((7-(1-carbamoyl-3-fluorocyclobutyl)-5-formylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate (28 mg, 0.05 mmol) in THF (0.5 mL) was added 3 M methylmagnesium bromide in Et₂O (89 μL, 0.27 mmol) at r.t. After stirring for 5 min, the reaction was quenched with sat. NH₄Cl, extracted with EtOAc (3×5 mL). The combined organic layers were subsequently washed with brine, dried over Na₂SO₄, filtered, and the solvents were evaporated in vacuo. The crude material was used in the next step without further purification. LCMS calculated for C₂₇H₂₉D₄FN₉O₅ (M+H)⁺: m/z=586.4; found 586.4.

### Step 4. Methyl ((1R,3R)-3-(6-((5-acetyl-7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

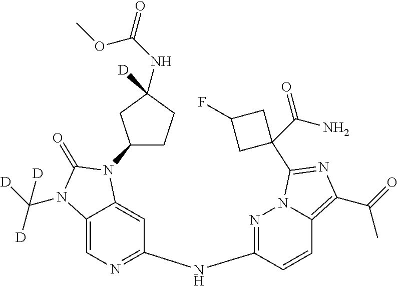 [SMILES: [2H]C([2H])([2H])n1c(=O)n([C@@H]2CC[C@@]([2H])(NC(=O)OC)C2)c2cc(Nc3ccc4c(C(C)=O)nc(C5(C(N)=O)CC(F)C5)n4n3)ncc21]

To a vial containing methyl ((1R,3R)-3-(6-((7-(1-carbamoyl-3-fluorocyclobutyl)-5-(1-hydroxyethyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate (60 mg, 0.1 mmol) in DCM (1 mL) was added 0.3 M of Dess-Martin periodinane in DCM (0.7 mL, 0.2 mmol) at r.t. After stirring for 30 min, the reaction was quenched with sat. NaHCO₃, extracted with DCM (3×5 mL). The combined organic layer was subsequently washed with brine, dried over Na₂SO₄, filtered, and the solvents were evaporated in vacuo. The crude material used in the next step without further purification. LCMS calculated for C₂₇H₂₇D₄FN₉O₅ (M+H)⁺: m/z=584.3; found 584.3.

### Step 5. Methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

To a vial containing methyl ((1R,3R)-3-(6-((5-acetyl-7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate (29 mg, 0.05 mmol) in THF (0.5 mL) was added 3 M methylmagnesium bromide in Et₂O (89 μL, 0.27 mmol) at r.t. After stirring for 5 min, the reaction was quenched with sat. NH₄Cl, extracted with EtOAc (3×5 mL). The combined organic layers were subsequently washed with brine, dried over Na₂SO₄, filtered, and the solvents were evaporated in vacuo. The crude material was diluted with acetonitrile and purified by prep-LCMS (XBridge C18 column, eluting with a gradient of acetonitrile/water containing 0.1% TFA, at flow rate of 60 mL/min). The desired product was obtained as a single diastereomer. LCMS calculated for C₂₈H₃₁D₄FN₉O₅ (M+H)⁺: m/z=600.2; found 600.2.

### Example B-12. Methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1,2,2,5,5-d₅)carbamate

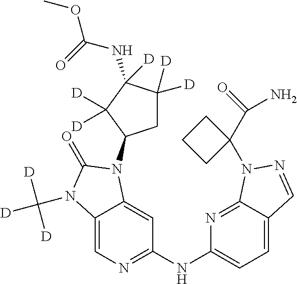 [SMILES: [2H]C([2H])([2H])n1c(=O)n([C@@H]2CC([2H])([2H])[C@@]([2H])(NC(=O)OC)C2([2H])[2H])c2cc(Nc3ccc4cnn(C5(C(N)=O)CCC5)c4n3)ncc21]

### Step 1. tert-Butyl (R)-(3-oxocyclopentyl-2,2,4,4-d₄)carbamate

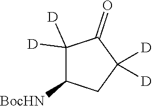 [SMILES: [2H]C1([2H])C[C@@H](NC(=O)OC(C)(C)C)C([2H])([2H])C1=O]

To a vial containing tert-butyl (R)-(3-oxocyclopentyl)carbamate (3.0 g, 15.1 mmol) in methanol-d₄ (10.0 mL) was added cesium carbonate (5 mg). After stirring at r.t. for 24 h, the reaction mixture was concentrated in vacuo. This process was repeated for two more times. After the third cycle, the reaction mixture was concentrated in vacuo. The crude product was used directly in the next step without further purification. ¹H NMR (400 MHz, CDCl₃) δ 4.22 (s, 1H), 2.35 (dd, J=13.1, 6.1 Hz, 1H), 1.85 (dd, J=13.1, 7.5 Hz, 1H), 1.47 (s, 9H).

### Step 2. Methyl ((1R,3R)-3-(6-amino-3-(methyl-d₃)-2-oxo-2,3-dihydro-1H-imidazo[4,5-c]pyridin-1-yl)cyclopentyl-1,2,2,5,5-d₅)carbamate

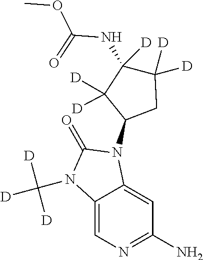 [SMILES: [2H]C([2H])([2H])n1c(=O)n([C@@H]2CC([2H])([2H])[C@@]([2H])(NC(=O)OC)C2([2H])[2H])c2cc(N)ncc21]

This compound was prepared according to the procedures described in Intermediate A, Step 1 to 9, with tert-butyl (R)-(3-oxocyclopentyl-2,2,4,4-d₄)carbamate replacing tert-butyl (R)-(3-oxocyclopentyl)carbamate in Step 1. LCMS calculated for C₁₄H₁₂D₈N₅O₃ (M+H)⁺: m/z=314.2; found: 314.2.

### Example B-13. Methyl ((1R,3R)-3-(6-((1-(1-carbamoylcyclobutyl)-4-(trans-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

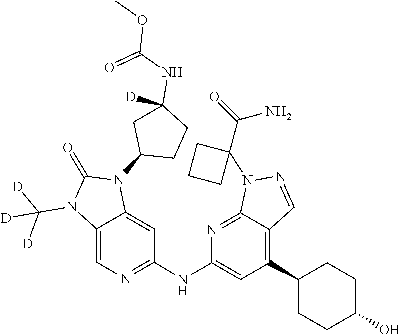 [SMILES: [2H]C([2H])([2H])n1c(=O)n([C@@H]2CC[C@@]([2H])(NC(=O)OC)C2)c2cc(Nc3cc([C@H]4CC[C@H](O)CC4)c4cnn(C5(C(N)=O)CCC5)c4n3)ncc21]

### Step 1. Ethyl 1-(6-chloro-4-(1,4-dioxaspiro[4.5]dec-7-en-8-yl)-1H-pyrazolo[3,4-b]pyridin-1-yl)cyclobutane-1-carboxylate

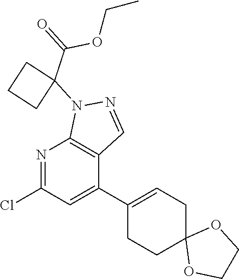 [SMILES: CCOC(=O)C1(n2ncc3c(C4=CCC5(CC4)OCCO5)cc(Cl)nc32)CCC1]

The mixture of ethyl 1-(6-chloro-4-iodo-1H-pyrazolo[3,4-b]pyridin-1-yl)cyclobutane-1-carboxylate (80 mg, 0.197 mmol), 4,4,5,5-tetramethyl-2-(1,4-dioxaspiro[4.5]dec-7-en-8-yl)-1,3,2-dioxaborolane (58 mg, 0.217 mmol), potassium phosphate tribasic (84 mg, 0.394 mmol) and (1,1′-bis(diphenylphosphino)ferrocene)-dichloropalladium(II) (24 mg, 0.030 mmol) in dioxane (0.4 ml) and water (0.1 ml) was evacuated and backfilled with nitrogen. The reaction was stirred at 60° C. for 2 hrs. The reaction was then cooled to r.t. and concentrated. The residue was purified by column chromatography eluted with EtOAc/hexane (0-50%) to obtain the desired compound. LCMS calculated for C₂₁H₂₅ClN₃O₄ (M+H)⁺: m/z=418.2; found: 418.2.

### Step 2. Ethyl 1-(6-((1-((1R,3R)-3-((methoxycarbonyl)amino)cyclopentyl-3-d)-3-(methyl-d)-2-oxo-2,3-dihydro-1H-imidazo[4,5-c]pyridin-6-yl)amino)-4-(1,4-dioxaspiro[4.5]dec-7-en-8-yl)-1H-pyrazolo[3,4-b]pyridin-1-yl)cyclobutane-1-carboxylate

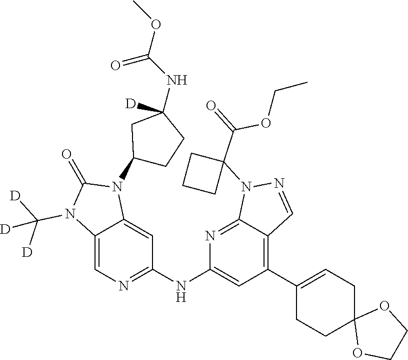 [SMILES: [2H]C([2H])([2H])n1c(=O)n([C@@H]2CC[C@@]([2H])(NC(=O)OC)C2)c2cc(Nc3cc(C4=CCC5(CC4)OCCO5)c4cnn(C5(C(=O)OCC)CCC5)c4n3)ncc21]

To a vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (74.0 mg, 0.167 mmol), Cs₂CO₃ (109 mg, 0.335 mmol), Pd₂(dba)₃ (30.7 mg, 0.033 mmol), xantphos (38.8 mg, 0.067 mmol) and ethyl 1-(6-chloro-4-(1,4-dioxaspiro[4.5]dec-7-en-8-yl)-1H-pyrazolo[3,4-b]pyridin-1-yl)cyclobutane-1-carboxylate (70.0 mg, 0.167 mmol) was added 1,4-dioxane (0.60 ml). The reaction was heated at 100° C. under N2 overnight. The reaction was then cooled to r.t., diluted with DCM and filtered over celite. The solvent was removed in vacuo and the crude was purified by column chromatography (MeOH/DCM, 0-30%) to obtain the desired product. LCMS calculated for C₃₅H₃₉D₄N₈O₇ (M+H)⁺: m/z=691.3; found: 691.5.

### Step 3. Ethyl 1-(6-((1-((1R,3R)-3-((methoxycarbonyl)amino)cyclopentyl-3-d)-3-(methyl-d)-2-oxo-2,3-dihydro-1H-imidazo[4,5-c]pyridin-6-yl)amino)-4-(1,4-dioxaspiro[4.5]decan-8-yl)-1H-pyrazolo[3,4-b]pyridin-1-yl)cyclobutane-1-carboxylate

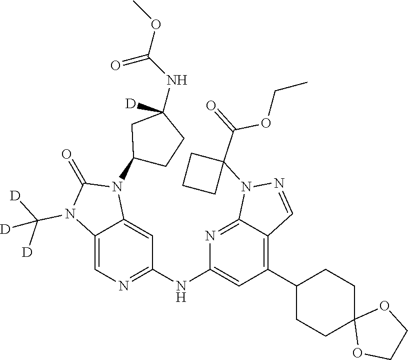 [SMILES: [2H]C([2H])([2H])n1c(=O)n([C@@H]2CC[C@@]([2H])(NC(=O)OC)C2)c2cc(Nc3cc(C4CCC5(CC4)OCCO5)c4cnn(C5(C(=O)OCC)CCC5)c4n3)ncc21]

To a vial containing ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(1,4-dioxaspiro[4.5]dec-7-en-8-yl)-1H-pyrazolo[3,4-b]pyridin-1-yl)cyclobutane-1-carboxylate (72 mg, 0.104 mmol) and palladium hydroxide on carbon (20 wt. %, 14.6 mg, 0.021 mmol) was added MeOH (5 mL). The mixture was stirred under H₂ balloon at 60° C. for 2 hrs. The reaction mixture was then cooled to r.t., filtered, and concentrated to obtain the desired product. LCMS calculated for C₃₅H₄₁D₄N₈O₇ (M+H)⁺: m/z=693.4; found: 693.5.

### Step 4. 1-(6-((1-((1R,3R)-3-((Methoxycarbonyl)amino)cyclopentyl-3-d)-3-(methyl-d₃)-2-oxo-2,3-dihydro-1H-imidazo[4,5-c]pyridin-6-yl)amino)-4-(4-oxocyclohexyl)-1H-pyrazolo[3,4-b]pyridin-1-yl)cyclobutane-1-carboxylic Acid

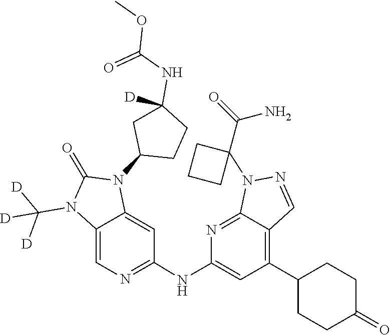 [SMILES: [2H]C([2H])([2H])n1c(=O)n([C@@H]2CC[C@@]([2H])(NC(=O)OC)C2)c2cc(Nc3cc(C4CCC(=O)CC4)c4cnn(C5(C(N)=O)CCC5)c4n3)ncc21]

To a solution of ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(1,4-dioxaspiro[4.5]decan-8-yl)-1H-pyrazolo[3,4-b]pyridin-1-yl)cyclobutane-1-carboxylate (64 mg, 0.092 mmol) in THF (1 ml), MeOH (1.000 ml) and water (0.2 ml) was added lithium hydroxide (11 mg, 0.462 mmol). The mixture was stirred at 60° C. for 30 min. The reaction was cooled to r.t. and quenched with HCl (0.077 ml, 0.924 mmol) and the resulting mixture was concentrated. The crude residue was dissolved in MeCN and purified by prep-LCMS (XBridge C18 column, eluting with a gradient of acetonitrile/water containing 0.1% TFA, at flow rate of 60 mL/min). LCMS calculated for C₃₁H₃₃D₄N₈O₆ (M+H)⁺: m/z=621.3; found: 621.3.

### Step 5. Methyl ((1R,3R)-3-(6-((1-(1-carbamoylcyclobutyl)-4-(4-oxocyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

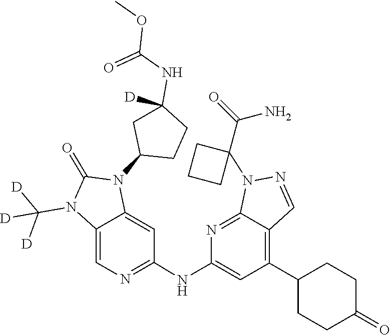 [SMILES: [2H]C([2H])([2H])n1c(=O)n([C@@H]2CC[C@@]([2H])(NC(=O)OC)C2)c2cc(Nc3cc(C4CCC(=O)CC4)c4cnn(C5(C(N)=O)CCC5)c4n3)ncc21]

To a vial containing 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-4-(4-oxocyclohexyl)-1H-pyrazolo[3,4-b]pyridin-1-yl)cyclobutane-1-carboxylic acid (50 mg, 0.075 mmol) and ammonia (0.5 M in dioxane, 0.752 mL, 0.376 mmol) as a solution in DMF (0.3 mL) and DIPEA (0.066 mL, 0.376 mmol) was added HATU (43 mg, 0.113 mmol) and left to stir at r.t. for 30 min. The crude residue was dissolved in MeCN and purified by prep-LCMS (XBridge C18 column, eluting with a gradient of acetonitrile/water containing 0.1% TFA, at flow rate of 60 mL/min). LCMS calculated for C₃₁H₃₄D₄N₉O₅ (M+H)⁺: m/z=620.3; found: 620.3.

### Step 6 Methyl ((1R,3R)-3-(6-((1-(1-carbamoylcyclobutyl)-4-((1r,4R)-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

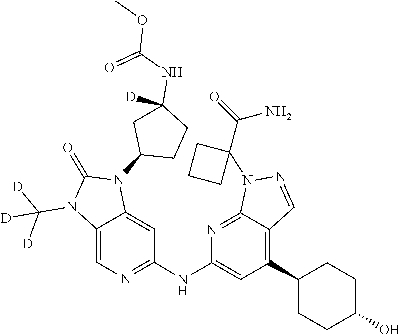 [SMILES: [2H]C([2H])([2H])n1c(=O)n([C@@H]2CC[C@@]([2H])(NC(=O)OC)C2)c2cc(Nc3cc([C@H]4CC[C@H](O)CC4)c4cnn(C5(C(N)=O)CCC5)c4n3)ncc21]

To a solution of methyl ((1R,3R)-3-(6-((1-(1-carbamoylcyclobutyl)-4-(4-oxocyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate (10 mg, 0.016 mmol) in MeOH (1.0 ml) was added sodium borohydride (0.7 mg, 0.019 mmol) at 0° C. and the reaction mixture was stirred at r.t. for 2 hrs. The crude residue was diluted with MeCN and purified by prep-LCMS (XBridge C18 column, eluting with a gradient of acetonitrile/water containing 0.1% TFA, at flow rate of 60 mL/min, the desired product was collected as major peak at Rt=11.1 min). LCMS calculated for C₃₁H₃₆D₄N₉O₅ (M+H)⁺: m/z=622.3; found: 622.3. ¹H NMR (600 MHz, DMSO-d₆) δ 8.34 (s, 1H), 8.24 (s, 1H), 7.43 (s, 1H), 7.37 (s, 1H), 6.79 (s, 1H), 5.03 (p, J=8.5 Hz, 1H), 3.60-3.52 (m, 1H), 3.55 (s, 3H), 2.97-2.81 (m, 5H), 2.36-2.30 (m, 1H), 2.24-2.11 (m, 2H), 2.09-1.91 (m, 8H), 1.69-1.59 (m, 3H), 1.46-1.37 (m, 2H) ppm.

### Example B-14. Methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

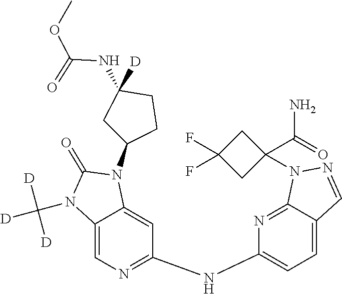 [SMILES: [2H]C([2H])([2H])n1c(=O)n([C@@H]2CC[C@@]([2H])(NC(=O)OC)C2)c2cc(Nc3ccc4cnn(C5(C(N)=O)CC(F)(F)C5)c4n3)ncc21]

### Step 1. Methyl 1-((6-bromo-3-cyanopyridin-2-yl)amino)-3,3-difluorocyclobutane-1-carboxylate

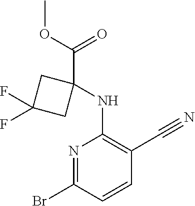 [SMILES: COC(=O)C1(Nc2nc(Br)ccc2C#N)CC(F)(F)C1]

To a reaction vial containing a solution of methyl 1-amino-3,3-difluorocyclobutane-1-carboxylate hydrochloride (176 mg, 0.87 mmol) and 6-bromo-2-fluoronicotinonitrile (175 mg, 0.87 mmol) in DMSO (0.3 mL) was added N,N-diisopropylethylamine (350 μL, 2.0 mmol). The reaction mixture was heated to 100° C. and allowed to stir for 10 h, after which it was cooled to r.t. and diluted with EtOAc. The organic layer was subsequently washed with water and brine, dried over sodium sulfate, filtered, and the solvents were evaporated in vacuo. The crude material was purified by Biotage Isolera to give the product. LCMS calculated for C₁₂H₁₁BrF₂N₃O₂ (M+H)⁺: m/z=346.0; found 346.0.

### Step 2. Methyl 1-(6-bromo-1H-pyrazolo[3,4-b]pyridin-1-yl)-3,3-difluorocyclobutane-1-carboxylate

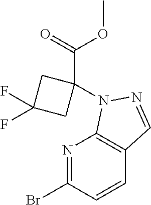 [SMILES: COC(=O)C1(n2ncc3ccc(Br)nc32)CC(F)(F)C1]

To a reaction vial containing a solution of methyl 1-((6-bromo-3-cyanopyridin-2-yl)amino)-3,3-difluorocyclobutane-1-carboxylate (205 mg, 0.6 mmol) in THF (0.8 mL) and DMF (1.6 mL) cooled to 0° C. was slowly added a lithium bis(trimethylsilyl)amide (770 μL, 0.77 mmol, 1 M in THF) and allowed to stir for 10 min. Then (aminooxy)diphenylphosphine oxide (138 mg, 0.6 mmol) was added and the reaction was allowed to warm to r.t. After 1 h at r.t, the reaction was quenched with a saturated aqueous solution of ammonium chloride and extracted with diethyl ether. The organic layers were combined, dried over sodium sulfate, filtered, and the solvents were evaporated in vacuo. The crude material was added to a reaction vial containing THF (0.8 mL) and tert-butyl nitrite (282 μL, 2.37 mmol). The reaction mixture was stirred at 65° C. for 2 h, after which the solvent was removed in vacuo, and the crude material was purified by Biotage Isolera to give the product. LCMS calculated for C₁₂H₁₁BrF₂N₃O₂ (M+H)⁺: m/z=346.0; found 346.0.

### Step 3. 3,3-Difluoro-1-(6-((1-((1R,3R)-3-((methoxycarbonyl)amino)cyclopentyl-3-d)-3-(methyl-d)-2-oxo-2,3-dihydro-1H-imidazo[4,5-c]pyridin-6-yl)amino)-1H-pyrazolo[3,4-b]pyridin-1-yl)cyclobutane-1-carboxylic Acid

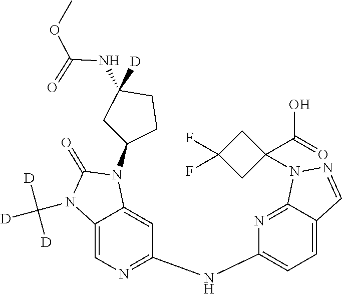 [SMILES: [2H]C([2H])([2H])n1c(=O)n([C@@H]2CC[C@@]([2H])(NC(=O)OC)C2)c2cc(Nc3ccc4cnn(C5(C(=O)O)CC(F)(F)C5)c4n3)ncc21]

To a reaction vial was added methyl ((1R,3R)-3-(6-amino-3-(methyl-d₃)-2-oxo-2,3-dihydro-1H-imidazo[4,5-c]pyridin-1-yl)cyclopentyl-1-d)carbamate (58 mg, 0.19 mmol, Intermediate A), methyl 1-(6-bromo-1H-pyrazolo[3,4-b]pyridin-1-yl)-3,3-difluorocyclobutane-1-carboxylate (65 mg, 0.19 mmol), XantPhos Pd G3 (18 mg, 19 μmol), and cesium carbonate (183 mg, 0.56 mmol). This was evacuated and backfilled with nitrogen 3 times. Subsequently 1,4-dioxane (2 mL) was added. The vial was sealed and heated to 100° C. for 2 h. Upon completion, the reaction was diluted with DCM, filtered through Celite and concentrated in vacuo. The crude residue was dissolved in a reaction vial containing THF (1 mL), MeOH (1 mL), and lithium hydroxide (1 mL, 1 M in H₂O), heated to 55° C., and allowed to stir for 4 h. Upon completion, the reaction was quenched with HCl (3 mL, 1 M in H₂O) and extracted with 3:1 chloroform:isopropanol. The organic layers were combined, dried over sodium sulfate, filtered, and the solvents were evaporated in vacuo. The crude material was used directly in the next reaction. LCMS calculated for C₂₅H₂₃D₄F₂N₈O₅ (M+H)⁺: m/z=561.2; found 561.2.

### Step 4. Methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a reaction vial containing 3,3-difluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylic acid (105 mg, 0.19 mmol), HATU (142 mg, 0.38 mmol) and DIPEA (100 μL, 0.56 mmol) was added ammonia (1.3 mL, 7 M in methanol). The reaction mixture was sealed and stirred at 60° C. for 2 h. Upon completion, the reaction was cooled to r.t. and solvent was removed in vacuo. The reaction residue was diluted with MeCN and purified by prep-LCMS (XBridge C18 column, eluting with a gradient 21→41% of acetonitrile/water containing 0.1% TFA, at flow rate of 60 mL/min.) Retention time=4.9 min LCMS calculated for C₂₅H₂₄D₄F₂N₉O₄ (M+H)⁺: m/z=560.3; found 560.3. ¹H NMR (600 MHz, DMSO) δ 11.35 (s, 1H), 8.28 (s, 1H), 8.25 (d, J=8.5 Hz, 1H), 8.21 (s, 1H), 7.68 (s, 1H), 7.59-7.38 (m, 2H), 7.32 (s, 1H), 7.04 (d, J=8.7 Hz, 1H), 5.05-4.96 (m, 1H), 3.66 (q, J=12.5 Hz, 4H), 3.54 (s, 3H), 2.34 (dd, J=13.7, 7.6 Hz, 1H), 2.24-2.11 (m, 2H), 2.10-2.01 (m, 1H), 1.95 (dd, J=13.7, 9.6 Hz, 1H), 1.68-1.60 (m, 1H).

### Example B-15. Methyl ((1R,3R)-3-(6-((1-(1-carbamoyl-3-methoxycyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

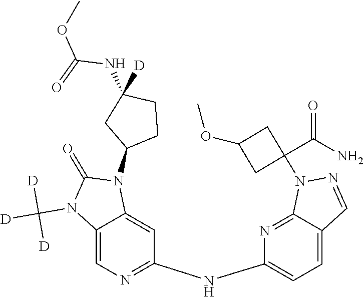 [SMILES: [2H]C([2H])([2H])n1c(=O)n([C@@H]2CC[C@@]([2H])(NC(=O)OC)C2)c2cc(Nc3ccc4cnn(C5(C(N)=O)CC(OC)C5)c4n3)ncc21]

This compound was prepared according to the procedures described in Example B-14, Steps 1-5 with methyl 1-amino-3-methoxycyclobutane-1-carboxylate hydrochloride replacing methyl 1-amino-3,3-difluorocyclobutane-1-carboxylate hydrochloride in Step 1, and final purification by prep-LCMS (XBridge C18 column, eluting with a gradient of 30→50% acetonitrile/aqueous 0.1% NH₄OH, at flow rate of 60 mL/min.) The product was obtained as a single diastereomer. The desired diastereomer was collected at a retention time of 3.3 min. LCMS calculated for C₂₆H₂₈D₄N₉O₅ (M+H)⁺: m/z=554.3; found 554.3.

### Example B-16. Methyl ((1R,3R)-3-(6-((1-(1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

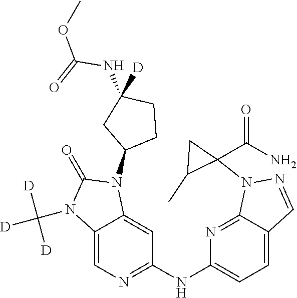 [SMILES: [2H]C([2H])([2H])n1c(=O)n([C@@H]2CC[C@@]([2H])(NC(=O)OC)C2)c2cc(Nc3ccc4cnn(C5(C(N)=O)CC5C)c4n3)ncc21]

This compound was prepared according to the procedures described in Example B-14, Steps 1-5 with rac-methyl (1R,2R)-1-amino-2-methylcyclopropane-1-carboxylate hydrochloride replacing methyl 1-amino-3,3-difluorocyclobutane-1-carboxylate hydrochloride in Step 1, and final purification by prep-LCMS (XBridge C18 column, eluting with a gradient of 28→48% acetonitrile/aqueous 0.1% NH₄OH, at flow rate of 60 mL/min.) The desired diastereomer was collected at a retention time of 4.2 min. LCMS calculated for C₂₅H₂₆D₄N₉O₄ (M+H)⁺: m/z=524.3; found 524.3. ¹H NMR (500 MHz, DMSO) δ 9.99 (s, 1H), 8.46 (s, 1H), 8.06 (s, 1H), 7.94 (d, J=8.7 Hz, 1H), 7.93 (s, 1H), 7.33 (s, 1H), 7.24 (s, 1H), 7.09 (d, J=8.7 Hz, 1H), 6.41 (s, 1H), 4.92-4.84 (m, 1H), 3.51 (s, 3H), 2.44 (dd, J=13.3, 7.5 Hz, 1H), 2.26-2.17 (m, 2H), 2.16-2.02 (m, 1H), 1.93 (dd, J=13.3, 9.3 Hz, 1H), 1.74 (dd, J=8.2, 4.8 Hz, 1H), 1.65 (m, 2H), 1.46 (dd, J=9.6, 4.7 Hz, 1H), 1.33 (d, J=6.3 Hz, 3H).

### Example B-17. Methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

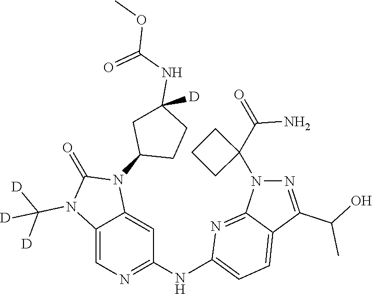 [SMILES: [2H]C([2H])([2H])n1c(=O)n([C@@H]2CC[C@@]([2H])(NC(=O)OC)C2)c2cc(Nc3ccc4c(C(C)O)nn(C5(C(N)=O)CCC5)c4n3)ncc21]

### Step 1. Ethyl 1-(6-bromo-1H-pyrazolo[3,4-b]pyridin-1-yl)cyclobutane-1-carboxylate

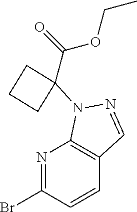 [SMILES: CCOC(=O)C1(n2ncc3ccc(Br)nc32)CCC1]

To a reaction flask containing a solution of ethyl 1-hydrazineylcyclobutane-1-carboxylate hydrochloride (3.8 g, 19.6 mmol) and 6-bromo-2-fluoronicotinaldehyde (4.0 g, 19.6 mmol) in N-methyl-2-pyrrolidone (30 mL) was added N,N-diisopropylethylamine (8.6 mL, 49.0 mmol). The reaction mixture was heated to 180° C. and allowed to stir for 3 h, after which it was cooled to r.t. and diluted with EtOAc. The organic layer was subsequently washed with an aqueous solution of lithium chloride and brine, dried over sodium sulfate, filtered, and the solvents were evaporated in vacuo. The crude material was purified by Biotage Isolera to give the product. LCMS calculated for C₁₃H₁₅BrN₃O₂ (M+H)⁺: m/z=324.0; found 324.0.

### Step 2. Ethyl 1-(6-bromo-3-iodo-1H-pyrazolo[3,4-b]pyridin-1-yl)cyclobutane-1-carboxylate

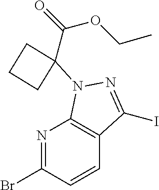 [SMILES: CCOC(=O)C1(n2nc(I)c3ccc(Br)nc32)CCC1]

To a flask containing ethyl 1-(6-bromo-1H-pyrazolo[3,4-b]pyridin-1-yl)cyclobutane-1-carboxylate (4.6 g, 14.3 mmol) in acetonitrile (100 mL) was added N-iodosuccinimide (3.5 g, 15.7 mmol) and tetrafluoroboric acid (9.3 mL, 71.3 mmol, 48% in H₂O). The reaction mixture was heated to 80° C. and stirred for 1 h, upon which it was cooled to r.t. and quenched with saturated aqueous solutions of NaHCO₃ and Na₂S₂O₃. The mixture was extracted with EtOAc and the organic layer was subsequently dried over sodium sulfate, filtered, and the solvents were evaporated in vacuo. The crude material was purified by Biotage Isolera to give the product. LCMS calculated for C₁₃H₁₄BrIN₃O₂ (M+H)⁺: m/z=449.9; found 449.9.

### Step 3. Ethyl 1-(6-bromo-3-vinyl-1H-pyrazolo[3,4-b]pyridin-1-yl)cyclobutane-1-carboxylate

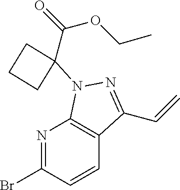 [SMILES: C=Cc1nn(C2(C(=O)OCC)CCC2)c2nc(Br)ccc12]

The reaction vial containing ethyl 1-(6-bromo-3-iodo-1H-pyrazolo[3,4-b]pyridin-1-yl)cyclobutane-1-carboxylate (1.0 g, 2.22 mmol), 4,4,5,5-tetramethyl-2-vinyl-1,3,2-dioxaborolane (0.4 mL, 2.33 mmol), cesium carbonate (1.1 g, 3.33 mmol) and [1,1′-bis(diphenylphosphino)ferrocene]-dichloropalladium(II) (363 mg, 0.44 mmol) was evacuated and backfilled with nitrogen. 1,4-Dioxane (6 mL) and water (1 mL) were added to the reaction mixture, which was then stirred at 55° C. for 8 h. The reaction mixture was cooled to r.t. and was diluted with EtOAc. The organic layer was subsequently washed with water and brine, dried over sodium sulfate, filtered, and the solvents were evaporated in vacuo. The crude material was purified by Biotage Isolera. LCMS calculated for: C₁₅H₁₇BrN₃O₂ (M+H)⁺: m/z=350.1; found 350.1.

### Step 4. Ethyl 1-(6-bromo-3-formyl-1H-pyrazolo[3,4-b]pyridin-1-yl)cyclobutane-1-carboxylate

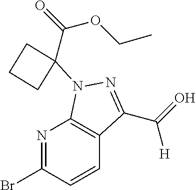 [SMILES: CCOC(=O)C1(n2nc(C=O)c3ccc(Br)nc32)CCC1]

A reaction vial containing ethyl 1-(6-bromo-3-vinyl-1H-pyrazolo[3,4-b]pyridin-1-yl)cyclobutane-1-carboxylate (546 mg, 1.56 mmol) in dichloromethane (6 mL) was cooled to −78° C. Ozone was bubbled through the solution until the solution turned light blue in color after 5 min. Dimethyl sulfide (0.58 mL, 7.8 mmol) was added to the solution, which was then allowed to warm to r.t. The solvents were removed in vacuo and the crude product was used directly in the next step without further purification. LCMS calculated for: C₁₄H₁₅BrN₃O₃ (M+H)⁺: m/z=352.0; found 352.0.

### Step 5. Ethyl 1-(6-bromo-3-(1-hydroxyethyl)-1H-pyrazolo[3,4-b]pyridin-1-yl)cyclobutane-1-carboxylate

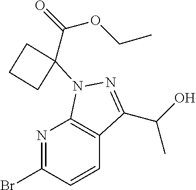 [SMILES: CCOC(=O)C1(n2nc(C(C)O)c3ccc(Br)nc32)CCC1]

To a reaction vial containing ethyl 1-(6-bromo-3-formyl-1H-pyrazolo[3,4-b]pyridin-1-yl)cyclobutane-1-carboxylate (549 mg, 1.56 mmol) in THF (4 mL) was added dropwise methylmagnesium bromide (0.52 mL, 1.56 mmol, 3.0 M in Et₂O) at −78° C. The reaction was allowed to warm to r.t. and stirred for 30 min. The mixture was quenched with sat. NH₄Cl and extracted with EtOAc. The organic layers were combined, dried over Na₂SO₄, filtered, and the solvents were evaporated in vacuo. The crude material was purified by Biotage Isolera. LCMS calculated for: C₁₅H₁₉BrN₃O₃ (M+H)⁺: m/z=368.1; found 368.1.

### Step 6 Methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

This compound was prepared according to the procedure in Example B-14, Steps 3-4, with ethyl 1-(6-bromo-3-(1-hydroxyethyl)-1H-pyrazolo[3,4-b]pyridin-1-yl)cyclobutane-1-carboxylate replacing methyl 1-(6-bromo-1H-pyrazolo[3,4-b]pyridin-1-yl)-3,3-difluorocyclobutane-1-carboxylate in Step 3. The final product was obtained as a mixture of two diastereomers. LCMS calculated for: C₂₇H₃₀D₄N₉O₅ (M+H)⁺: m/z=568.3; found 568.3.

### Example B-18. Methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

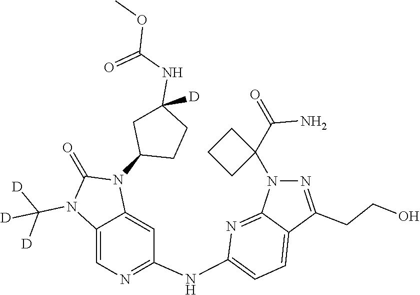 [SMILES: [2H]C([2H])([2H])n1c(=O)n([C@@H]2CC[C@@]([2H])(NC(=O)OC)C2)c2cc(Nc3ccc4c(CCO)nn(C5(C(N)=O)CCC5)c4n3)ncc21]

### Step 1. Ethyl (E)-1-(6-bromo-3-(2-ethoxyvinyl)-1H-pyrazolo[3,4-b]pyridin-1-yl)cyclobutane-1-carboxylate

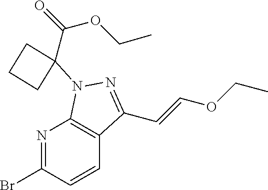 [SMILES: CCO/C=C/c1nn(C2(C(=O)OCC)CCC2)c2nc(Br)ccc12]

This compound was prepared according to the procedure in Example B-17, Step 3, with (E)-2-(2-ethoxyvinyl)-4,4,5,5-tetramethyl-1,3,2-dioxaborolane replacing 4,4,5,5-tetramethyl-2-vinyl-1,3,2-dioxaborolane. LCMS calculated for: C₁₇H₂₁BrN₃O₃ (M+H)⁺: m/z=394.1; found 394.1.

### Step 2. Ethyl 1-(3-((E)-2-ethoxyvinyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

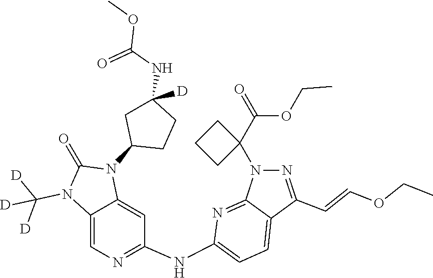 [SMILES: [2H]C([2H])([2H])n1c(=O)n([C@@H]2CC[C@@]([2H])(NC(=O)OC)C2)c2cc(Nc3ccc4c(/C=C/OCC)nn(C5(C(=O)OCC)CCC5)c4n3)ncc21]

To a reaction vial was added methyl ((1R,3R)-3-(6-amino-3-(methyl-d₃)-2-oxo-2,3-dihydro-1H-imidazo[4,5-c]pyridin-1-yl)cyclopentyl-1-d)carbamate (190 mg, 0.61 mmol, Intermediate A), ethyl (E)-1-(6-bromo-3-(2-ethoxyvinyl)-1H-pyrazolo[3,4-b]pyridin-1-yl)cyclobutane-1-carboxylate (242 mg, 0.61 mmol), tris(dibenzylideneacetone)dipalladium (84 mg, 92 μmol), 9,9-dimethyl-9H-xanthene-4,5-diyl)bis(diphenylphosphane (107 mg, 0.18 mmol), and cesium carbonate (600 mg, 1.84 mmol). This was evacuated and backfilled with nitrogen 3 times. Subsequently 1,4-dioxane (3 mL) was added. The vial was sealed and heated to 100° C. for 2 h. Upon completion, the reaction was cooled to r.t., diluted with DCM, filtered through Celite and concentrated in vacuo. The organic layers were combined, dried over sodium sulfate, filtered, and the solvents were evaporated in vacuo. The crude material was purified by Biotage Isolera to give the product. LCMS calculated for: C₃₁H₃₅D₄N₈O₆ (M+H)⁺: m/z=623.3; found 623.3.

### Step 3. Ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(2-oxoethyl)-1H-pyrazolo[3,4-b]pyridin-1-yl)cyclobutane-1-carboxylate

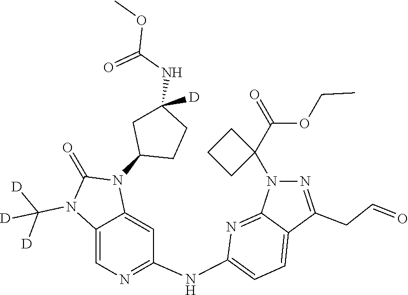 [SMILES: [2H]C([2H])([2H])n1c(=O)n([C@@H]2CC[C@@]([2H])(NC(=O)OC)C2)c2cc(Nc3ccc4c(CC=O)nn(C5(C(=O)OCC)CCC5)c4n3)ncc21]

To a reaction vial containing ethyl 1-(3-((E)-2-ethoxyvinyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (322 mg, 0.52 mmol) in dichloromethane (2 mL) was added trifluoroacetic acid (2 mL). The reaction was heated to 35° C. and allowed to stir for 2 h, upon which the solvents were removed in vacuo. The crude residue was extracted with dichloromethane and washed with saturated aqueous NaHCO₃. The organic layers were combined, dried over Na₂SO₄, filtered, and solvents were removed in vacuo. The crude product was used directly in the next step without further purification. LCMS calculated for: C₂₉H₃₁D₄N₈O₆ (M+H)⁺: m/z=595.3; found 595.3.

### Step 4. Ethyl 1-(3-(2-hydroxyethyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

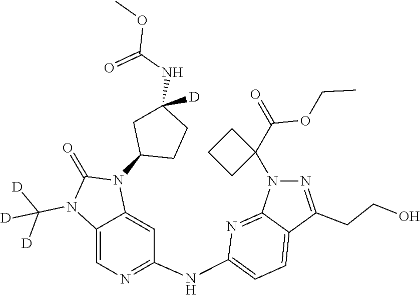 [SMILES: [2H]C([2H])([2H])n1c(=O)n([C@@H]2CC[C@@]([2H])(NC(=O)OC)C2)c2cc(Nc3ccc4c(CCO)nn(C5(C(=O)OCC)CCC5)c4n3)ncc21]

To a via containing ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(2-oxoethyl)-1H-pyrazolo[3,4-b]pyridin-1-yl)cyclobutane-1-carboxylate (25 mg, 42 μmol) in methanol (0.5 mL) was added sodium borohydride (8 mg, 0.2 mmol). After stirring at r.t. for 30 min, the reaction mixture was quenched with water, and extracted with EtOAc. The organic layers were combined, dried over Na₂SO₄, filtered, and solvents were removed in vacuo. The crude material was purified by Biotage Isolera to give the product. LCMS calculated for: C₂₉H₃₃D₄N₈O₆ (M+H)⁺: m/z=597.3; found 597.3.

### Step 5. Methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a reaction vial containing ethyl 1-(3-(2-hydroxyethyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (25 mg, 42 μmol) in THF (1 mL) and MeOH (1 mL) was added lithium hydroxide (1 mL, 1 M in H₂O). The reaction mixture was heated to 50° C. and allowed to stir for 1 h, after which the reaction was quenched with HCl (3 mL, 1 M in H₂O) and extracted with 3:1 chloroform:isopropanol. The organic layers were combined, dried over sodium sulfate, filtered, and the solvents were evaporated in vacuo. The crude material was added to a reaction vial containing HATU (24 mg, 63 μmol) and DIPEA (22 μL, 0.13 mmol) in DMF (1 mL). Ammonia (1.3 mL, 7 M in methanol) was added to this reaction mixture, and it was then sealed and stirred at r.t. for 1 h. Upon completion, a nitrogen gas inlet needle was inserted to blow off excess ammonia. The reaction residue was diluted with MeCN and purified by prep-LCMS (XBridge C18 column, eluting with a gradient 21→41% of acetonitrile/water containing 0.1% TFA, at flow rate of 60 mL/min.) Retention time=3.9 min. LCMS calculated for: C₂₇H₃₀D₄N₉O₅ (M+H)⁺: m/z=568.3; found 568.3. ¹H NMR (600 MHz, DMSO) δ 11.51 (s, 1H), 8.29 (s, 1H), 8.28 (s, 1H), 7.46 (s, 1H), 7.43-7.26 (m, 3H), 6.93 (d, J=8.6 Hz, 1H), 5.07-4.98 (m, 1H), 3.82 (t, J=6.7 Hz, 2H), 3.55 (s, 3H), 3.08 (t, J=6.7 Hz, 2H), 2.95-2.81 (m, 4H), 2.33 (dd, J=13.7, 7.8 Hz, 1H), 2.25-2.11 (m, 2H), 2.10-2.00 (m, 1H), 2.00-1.90 (m, 3H), 1.69-1.62 (m, 1H).

### Example B-19. Methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d)-2-oxo-2,3-dihydro-1H-imidazo[4,5-c]pyridin-1-yl)cyclopentyl-1-d)carbamate

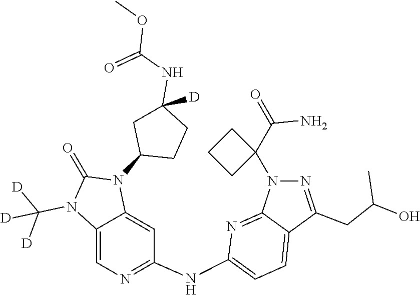 [SMILES: [2H]C([2H])([2H])n1c(=O)n([C@@H]2CC[C@@]([2H])(NC(=O)OC)C2)c2cc(Nc3ccc4c(CC(C)O)nn(C5(C(N)=O)CCC5)c4n3)ncc21]

### Step 1. Ethyl 1-(3-(2-hydroxypropyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

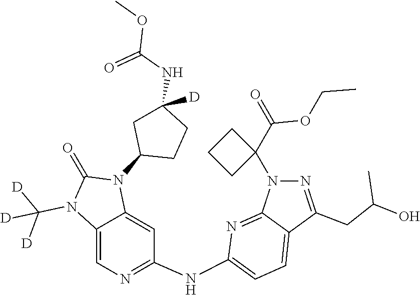 [SMILES: [2H]C([2H])([2H])n1c(=O)n([C@@H]2CC[C@@]([2H])(NC(=O)OC)C2)c2cc(Nc3ccc4c(CC(C)O)nn(C5(C(=O)OCC)CCC5)c4n3)ncc21]

This compound was prepared according to the procedure in Example B-17, Step 5, with ethyl 1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-3-(2-oxoethyl)-1H-pyrazolo[3,4-b]pyridin-1-yl)cyclobutane-1-carboxylate replacing ethyl 1-(6-bromo-3-formyl-1H-pyrazolo[3,4-b]pyridin-1-yl)cyclobutane-1-carboxylate. LCMS calculated for: C₃₀H₃₅D₄N₈O₆ (M+H)⁺: m/z=611.3; found 611.3.

### Step 2. Methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

This compound was prepared according to the procedure in Example B-18, Step 5, with ethyl 1-(3-(2-hydroxypropyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate replacing ethyl 1-(3-(2-hydroxyethyl)-6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate. The final product was obtained as a mixture of diastereomers. LCMS calculated for: C₂₈H₃₂D₄N₉O₅ (M+H)⁺: m/z=582.3; found 582.3.

### Example B-20. Methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

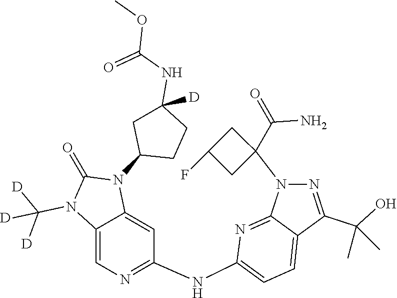 [SMILES: [2H]C([2H])([2H])n1c(=O)n([C@@H]2CC[C@@]([2H])(NC(=O)OC)C2)c2cc(Nc3ccc4c(C(C)(C)O)nn(C5(C(N)=O)CC(F)C5)c4n3)ncc21]

### Step 1. Isopropyl 1-(6-bromo-3-(prop-1-en-2-yl)-1H-pyrazolo[3,4-b]pyridin-1-yl)-3-fluorocyclobutane-1-carboxylate

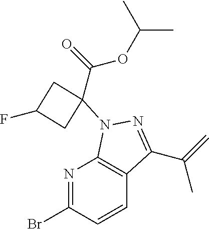 [SMILES: C=C(C)c1nn(C2(C(=O)OC(C)C)CC(F)C2)c2nc(Br)ccc12]

This compound was prepared according to the procedure in Example B-17, Steps 2-3, with isopropyl 1-(6-bromo-1H-pyrazolo[3,4-b]pyridin-1-yl)-3-fluorocyclobutane-1-carboxylate (Example B-24, Step 6) replacing ethyl 1-(6-bromo-1H-pyrazolo[3,4-b]pyridin-1-yl)cyclobutane-1-carboxylate in Step 2, and with 4,4,5,5-tetramethyl-2-(prop-1-en-2-yl)-1,3,2-dioxaborolane replacing 4,4,5,5-tetramethyl-2-vinyl-1,3,2-dioxaborolane in Step 3. LCMS calculated for: C₁₇H₂₀BrFN₃O₂ (M+H)⁺: m/z=396.1; found 396.1.

### Step 2. Isopropyl 1-(6-bromo-3-(2-hydroxypropan-2-yl)-1H-pyrazolo[3,4-b]pyridin-1-yl)-3-fluorocyclobutane-1-carboxylate

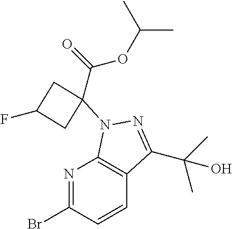 [SMILES: CC(C)OC(=O)C1(n2nc(C(C)(C)O)c3ccc(Br)nc32)CC(F)C1]

To a reaction vial containing isopropyl 1-(6-bromo-3-(prop-1-en-2-yl)-1H-pyrazolo[3,4-b]pyridin-1-yl)-3-fluorocyclobutane-1-carboxylate (55 mg, 0.14 mmol), Fe(acac)₃ (5 mg, 14 μmmol), methyl 4-nitrobenzenesulfonate (45 mg, 0.21 mmol), and NaHCO₃ (35 mg, 0.42 mmol) was added methanol (1 mL). The reaction mixture was cooled to 0° C. and allowed to stir for 5 min, after which phenylsilane (21 μL, 0.17 mmol) was added dropwise and the reaction was allowed to stir open to air at r.t. for 1 h. The solvents were removed in vacuo and the crude material was purified by Biotage Isolera. LCMS calculated for: C₁₇H₂₂BrFN₃O₃ (M+H)⁺: m/z=414.1; found 414.1.

### Step 3. Methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

This compound was prepared according to the procedure in Example B-14, Steps 3-4, with isopropyl 1-(6-bromo-3-(2-hydroxypropan-2-yl)-1H-pyrazolo[3,4-b]pyridin-1-yl)-3-fluorocyclobutane-1-carboxylate replacing methyl 1-(6-bromo-1H-pyrazolo[3,4-b]pyridin-1-yl)-3,3-difluorocyclobutane-1-carboxylate in Step 3, and final purification by prep-LCMS (XBridge C18 column, eluting with a gradient of 35→55% acetonitrile/aqueous 0.1% NH₄OH, at flow rate of 60 mL/min.). The product was isolated as a single isomer. Retention time=3.2. LCMS calculated for: C₂₈H₃₁D₄FN₉O₅ (M+H)⁺: m/z=600.3; found 600.3.

### Example B-21. Methyl ((1R,3R)-3-(6-((3-(1-carbamoyl-3-fluorocyclobutyl)-7-methyl-2-(trifluoromethyl)-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

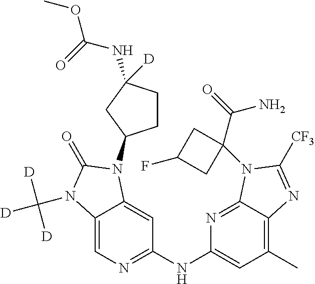 [SMILES: [2H]C([2H])([2H])n1c(=O)n([C@@H]2CC[C@@]([2H])(NC(=O)OC)C2)c2cc(Nc3cc(C)c4nc(C(F)(F)F)n(C5(C(N)=O)CC(F)C5)c4n3)ncc21]

This compound was prepared according to the procedures described in Example B-22, Step 1 to 8, with trifluoroacetic anhydride replacing 2,2-difluoroacetic anhydride in Step 3. The crude product was purified by prep-LCMS as the second peak of two diastereoisomers (XBridge C18 column, eluting with a gradient of 16.9→34.8% acetonitrile in water containing 0.1% TFA, at flow rate of 60 mL/min over 12 min). The desired product was collected at a retention time of 10.9 min as a single diastereomer. LCMS calculated for C₂₇H₂₆D₄F₄N₉O₄ (M+H)⁺: m/z=624.3; found 624.3.

### Example B-22. Methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

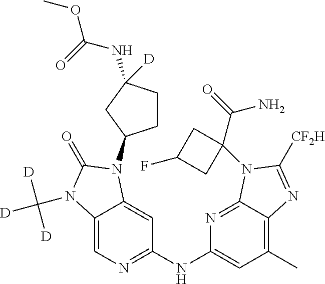 [SMILES: [2H]C([2H])([2H])n1c(=O)n([C@@H]2CC[C@@]([2H])(NC(=O)OC)C2)c2cc(Nc3cc(C)c4nc(C(F)F)n(C5(C(N)=O)CC(F)C5)c4n3)ncc21]

### Step 1. (1-((6-Chloro-4-methyl-3-nitropyridin-2-yl)amino)-3-fluorocyclobutyl)methanol

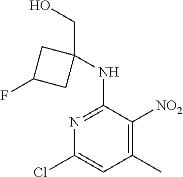 [SMILES: Cc1cc(Cl)nc(NC2(CO)CC(F)C2)c1[N+](=O)[O-]]

To a vial containing 2,6-dichloro-4-methyl-3-nitropyridine (270 mg, 1.28 mmol) and (1-amino-3-fluorocyclobutyl)methanol hydrochloride (200 mg, 1.28 mmol) in DMSO (0.43 mL) was added DIPEA (450 μL, 2.6 mmol). After heating at 65° C. for 12 h, the reaction mixture was directly purified by Biotage Isolera to give the desired product as yellow solid. LCMS calculated for C₁₁H₁₄ClFN₃O₃ (M+H)⁺: m/z=290.1; found 290.1.

### Step 2. (1-((3-Amino-6-chloro-4-methylpyridin-2-yl)amino)-3-fluorocyclobutyl)methanol

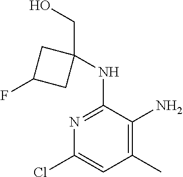 [SMILES: Cc1cc(Cl)nc(NC2(CO)CC(F)C2)c1N]

A vial containing a suspension of zinc dust (590 mg, 9.0 mmol) and NH₄Cl (480 mg, 9.0 mmol) in MeOH (10 mL) was placed in a water bath. A solution of (1-((6-chloro-4-methyl-3-nitropyridin-2-yl)amino)-3-fluorocyclobutyl)methanol (260 mg, 0.90 mmol) in CH₂Cl₂ (2 mL) was added dropwise to the zinc powder suspension with vigorous stirring at the rate of 2 drop/sec. After stirring at r.t for 20 min, the reaction mixture was filtered and washed with CH₂Cl₂ (8 mL) three times. The filtrate was collected and concentrated. The residue was dissolved in CH₂Cl₂ (20 mL), washed with water (5 mL), and extracted with CH₂Cl₂. The organic phase was washed with brine, dried over MgSO₄ and concentrated in vacuo. The obtained crude product was used in the next step without further purification. LCMS calculated for C₁₁H₁₆ClFN₃₀ (M+H)⁺: m/z=260.1; found 260.1.

### Step 3. N-(6-Chloro-2-((3-fluoro-1-(hydroxymethyl)cyclobutyl)amino)-4-methylpyridin-3-yl)-2,2-difluoroacetamide

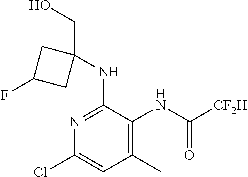 [SMILES: Cc1cc(Cl)nc(NC2(CO)CC(F)C2)c1NC(=O)C(F)F]

To a vial containing (1-((3-amino-6-chloro-4-methylpyridin-2-yl)amino)-3-fluorocyclobutyl)methanol (100 mg, 0.38 mmol) in CH₂Cl₂ (2.0 mL) was added DIPEA (0.34 mL, 1.93 mmol) and 2,2-difluoroacetic anhydride (200 mg, 1.16 mmol). After stirring at r.t for 20 min, the reaction mixture was quenched with MeOH (1.0 mL) and concentrated in vacuo. The crude mixture was dissolved in MeOH/THF/water (1.5 mL, v/v/v=1/1/1), followed by the addition of LiOH (18 mg, 0.78 mmol). After heating at 50° C. for 15 min, the reaction mixture was cooled to r.t. and extracted with CH₂Cl₂ (3×5 mL). The organic layers were combined, dried over MgSO₄ and concentrated in vacuo. The obtained crude product was used in the next step without further purification. LCMS calculated for C₁₃H₁₆ClF₃N₃O₂ (M+H)⁺: m/z=338.1; found 338.1.

### Step 4. (1-(5-Chloro-2-(difluoromethyl)-7-methyl-3H-imidazo[4,5-b]pyridin-3-yl)-3-fluorocyclobutyl)methanol

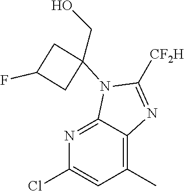 [SMILES: Cc1cc(Cl)nc2c1nc(C(F)F)n2C1(CO)CC(F)C1]

To a vial containing crude N-(6-chloro-2-((3-fluoro-1-(hydroxymethyl)cyclobutyl)-amino)-4-methylpyridin-3-yl)-2,2-difluoroacetamide (130 mg, 0.38 mmol) was added acetic acid (1.0 ml). After stirring at 100° C. for 14 h, the reaction mixture was diluted with EtOAc (3 mL) quenched with sat. aq. NaHCO₃ solution, followed by extraction with EtOAc (3×10 mL). The organic layers were combined, dried over MgSO₄ and concentrated in vacuo. The crude mixture was dissolved in MeOH/THF/water (1.5 mL, v/v/v=1/1/1), followed by the addition of LiOH (18 mg, 0.78 mmol). After heating at 50° C. for 15 min, the reaction mixture was cooled to r.t. and extracted with CH₂Cl₂ (3×5 mL). The organic layer was combined, dried over MgSO₄ and concentrated in vacuo. The obtained crude product was used in the next step without further purification. LCMS calculated for C₁₃H₁₄ClF₃N₃O (M+H)⁺: m/z=320.1; found 320.2.

### Step 5. 1-(5-Chloro-2-(difluoromethyl)-7-methyl-3H-imidazo[4,5-b]pyridin-3-yl)-3-fluorocyclobutane-1-carboxylic Acid

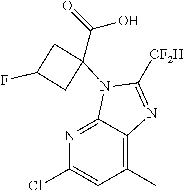 [SMILES: Cc1cc(Cl)nc2c1nc(C(F)F)n2C1(C(=O)O)CC(F)C1]

To a vial containing (1-(5-chloro-2-(difluoromethyl)-7-methyl-3H-imidazo[4,5-b]pyridin-3-yl)-3-fluorocyclobutyl)methanol (121 mg, 0.38 mmol) in CH₂Cl₂/EtOAc/water (3.0 mL, v/v/v=1/1/1) was added sodium periodate (250 mg, 1.16 mmol) and ruthenium(III) chloride (1 mg, 4 μmol). The reaction mixture was stirred vigorously and heated at 45° C. for 40 min. The reaction mixture was cooled down to r.t. and extracted with EtOAc (3×4 mL). The organic layers were combined, dried over MgSO₄ and concentrated in vacuo. The obtained crude product was used in the next step without further purification. LCMS calculated for C₁₃H₁₂ClF₃N₃O₂ (M+H)⁺: m/z=334.0; found 334.1.

### Step 6 Methyl 1-(5-chloro-2-(difluoromethyl)-7-methyl-3H-imidazo[4,5-b]pyridin-3-yl)-3-fluorocyclobutane-1-carboxylate

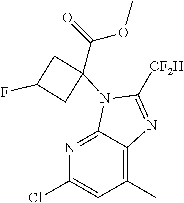 [SMILES: COC(=O)C1(n2c(C(F)F)nc3c(C)cc(Cl)nc32)CC(F)C1]

To a vial containing 1-(5-chloro-2-(difluoromethyl)-7-methyl-3H-imidazo[4,5-b]pyridin-3-yl)-3-fluorocyclobutane-1-carboxylic acid (127 mg, 0.38 mmol) and potassium carbonate (106 mg, 0.77 mmol) in acetonitrile (2.0 mL) was added iodomethane (72 μL, 1.16 mmol). The reaction mixture was heated at 50° C. for 1 h. The reaction mixture was cooled down to r.t., quenched with sat. aq. NH₄Cl solution (3 mL) and extracted with EtOAc (3×4 mL). The organic layers were combined, dried over MgSO₄ and concentrated in vacuo. The crude material was purified by Biotage Isolera to give the desired product as white solid. LCMS calculated for C₁₄H₁₄ClF₃N₃O₂ (M+H)⁺: m/z=348.1; found 348.1.

### Step 7. 1-(2-(Difluoromethyl)-5-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-7-methyl-3H-imidazo[4,5-b]pyridin-3-yl)-3-fluorocyclobutane-1-carboxylic Acid

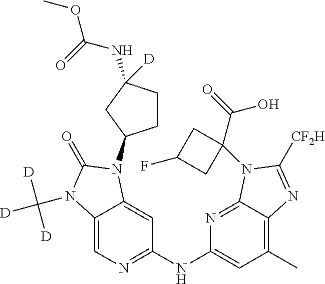 [SMILES: [2H]C([2H])([2H])n1c(=O)n([C@@H]2CC[C@@]([2H])(NC(=O)OC)C2)c2cc(Nc3cc(C)c4nc(C(F)F)n(C5(C(=O)O)CC(F)C5)c4n3)ncc21]

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (21 mg, 67 μmol, Intermediate A), methyl 1-(5-chloro-2-(difluoromethyl)-7-methyl-3H-imidazo[4,5-o]pyridin-3-yl)-3-fluorocyclobutane-1-carboxylate (23 mg, 67 μmol), tris(dibenzylideneacetone)-dipalladium(0) (9 mg, 9.6 μmol), 4,5-bis(diphenylphosphino)-9,9-dimethylxanthene (11 mg, 19 μmol) and cesium carbonate (44 mg, 134 μmol) was evacuated and backfilled with nitrogen three times, followed by the addition of 1,4-dioxane (0.6 mL). The vial was sealed and heated to 100° C. for 16 h. After cooling to r.t., the mixture was diluted with MeOH (0.6 mL) and water (0.6 mL), followed by the addition of LiOH (16 mg, 0.67 mmol). After heating at 50° C. for 15 min, the reaction mixture was cooled to r.t. and diluted with CH₂Cl₂ (3 mL). The pH of the mixture was adjusted to −4 with HCl aqueous solution, followed by the extraction with CH₂Cl₂ (3×5 mL). The organic layers were combined, dried over MgSO₄ and concentrated in vacuo. The obtained crude product was used in the next step without further purification. LCMS calculated for C₂₇H₂₆D₄F₃N₈O₅ (M+H)⁺: m/z=607.2; found 607.2.

### Step 8. Methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d)-2-oxo-2,3-dihydro-1H-imidazo[4,5-c]pyridin-1-yl)cyclopentyl-1-d)carbamate

To a vial containing crude 1-(2-(difluoromethyl)-5-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-7-methyl-3H-imidazo[4,5-b]pyridin-3-yl)-3-fluorocyclobutane-1-carboxylic acid from Step 7 was added thionyl chloride (1.0 mL). The vial was heated to 50° C. for 30 min. After cooling to r.t., the reaction mixture was concentrated in vacuo to remove thionyl chloride. The crude acid chloride was dissolved in THF (2.0 mL) and added dropwise to the stirring ammonia aqueous solution (5.0 mL) at 0° C. After the addition of the acid chloride solution, the reaction mixture was extracted into CH₂Cl₂. The organic layers were combined, dried over MgSO₄ and concentrated in vacuo. The crude product was purified by prep-LCMS as the second peak of two diastereoisomers (XBridge C18 column, eluting with a gradient of 15.9→30.4% acetonitrile in water containing 0.1% TFA, at flow rate of 60 mL/min over 12 min). The desired product was collected at a retention time of 11.1 min as a single diastereomer. LCMS calculated for C₂₇H₂₇D₄F₃N₉O₄ (M+H)⁺: m/z=606.3; found 606.2.

### Example B-23. Methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

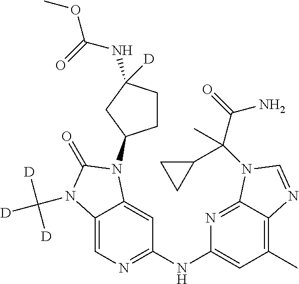 [SMILES: [2H]C([2H])([2H])n1c(=O)n([C@@H]2CC[C@@]([2H])(NC(=O)OC)C2)c2cc(Nc3cc(C)c4ncn(C(C)(C(N)=O)C5CC5)c4n3)ncc21]

### Step 1. 2-((3-Amino-6-chloro-4-methylpyridin-2-yl)amino)-2-cyclopropylpropan-1-ol

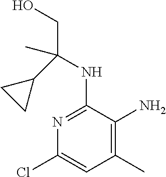 [SMILES: Cc1cc(Cl)nc(NC(C)(CO)C2CC2)c1N]

This compound was prepared according to the procedures described in Example B-22, Step 1 to 2, with 2-amino-2-cyclopropylpropan-1-ol replacing (1-amino-3-fluorocyclobutyl)methanol hydrochloride in Step 1. The obtained crude product was used in the next step without further purification as a racemic mixture. LCMS calculated for C₁₂H₁₉ClN₃O (M+H)⁺: m/z=256.1; found 256.1.

### Step 2. 2-(5-Chloro-7-methyl-3H-imidazo[4,5-b]pyridin-3-yl)-2-cyclopropylpropan-1-ol

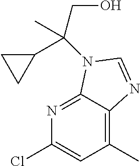 [SMILES: Cc1cc(Cl)nc2c1ncn2C(C)(CO)C1CC1]

To a vial containing 2-((3-amino-6-chloro-4-methylpyridin-2-yl)amino)-2-cyclopropylpropan-1-ol (100 mg, 0.39 mmol) in triethyl orthoformate (590 mg, 0.66 mL, 3.9 mmol) was added p-toluenesulfonic acid monohydrate (1 mg). The reaction mixture was heated at 90° C. for 30 min. The reaction mixture was cooled down to r.t. and quenched with sat. aq. NH₄Cl solution (3 mL) and extracted with CH₂Cl₂. The organic layers were combined, dried over MgSO₄ and concentrated in vacuo. The obtained crude product was used in the next step without further purification. LCMS calculated for C₁₃H₁₇ClN₃O (M+H)⁺: m/z=266.1; found 266.1.

### Step 3. Methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

This compound was prepared according to the procedures described in Example B-22, Step 5 to 8, with 2-(5-chloro-7-methyl-3H-imidazo[4,5-b]pyridin-3-yl)-2-cyclopropylpropan-1-ol replacing (1-(5-chloro-2-(difluoromethyl)-7-methyl-3H-imidazo[4,5-b]pyridin-3-yl)-3-fluorocyclobutyl)methanol in Step 5. The crude product was purified by prep-LCMS (XBridge C18 column, eluting with a gradient of acetonitrile/water containing 0.1% TFA, at flow rate of 60 mL/min). The final product was obtained as a mixture of two diastereomers. LCMS calculated for C₂₇H₃₀D₄N₉O₄ (M+H)⁺: m/z=552.3; found 552.3.

### Example B-24. Methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

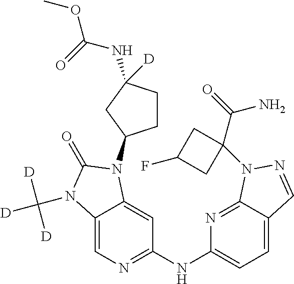 [SMILES: [2H]C([2H])([2H])n1c(=O)n([C@@H]2CC[C@@]([2H])(NC(=O)OC)C2)c2cc(Nc3ccc4cnn(C5(C(N)=O)CC(F)C5)c4n3)ncc21]

### Step 1. 3-Fluoro-1-(isopropoxycarbonyl)cyclobutane-1-carboxylic Acid

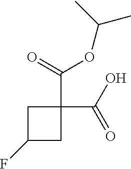

A round-bottom flask containing a solution of di-iso-propyl 3-fluorocyclobutane-1,1-dicarboxylate (16.0 g, 65.0 mmol) in iso-propanol (50 mL) was placed in an ice bath at 0° C. A solution of potassium hydroxide (3.65 g, 65.0 mmol) in iso-propanol (50 mL) and water (25 mL) was added via addition funnel to the di-ester solution at the rate of 1 drop/sec. The reaction mixture was allowed to warm up to r.t. after the addition of potassium hydroxide solution. After stirring at r.t for 16 h, the reaction mixture was concentrated in vacuo to remove iso-propanol, and then acidified with hydrochloric acid. The resulting solution was extracted with EtOAc three times. The organic phase was washed with brine, dried over MgSO₄, and concentrated in vacuo. The crude mixture was azeotroped with of toluene (30 mL) for three times to remove the remaining iso-propanol in the residue. The resulting colorless oil was used in the next step without further purification. ¹H NMR (400 MHz, CDCl₃) δ 5.32-4.98 (m, 2H), 3.06-2.66 (m, 4H), 1.36-1.22 (m, 6H).

### Step 2. Isopropyl 1-(((benzyloxy carbonyl)amino)-3-fluorocyclobutane-1-carboxylate

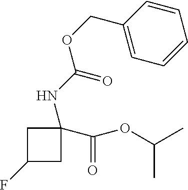 [SMILES: CC(C)OC(=O)C1(NC(=O)OCc2ccccc2)CC(F)C1]

To a vial containing 3-fluoro-1-(isopropoxycarbonyl)cyclobutane-1-carboxylic acid (1.0 g, 4.9 mmol) in toluene (20 mL) was added triethylamine (0.78 mL, 5.6 mmol) and diphenylphosphoryl azide (1.16 mL, 5.40 mmol). After heating at 50° C. for 20 min, the reaction mixture was cooled down to r.t., followed by the addition of phenylmethanol (0.69 g, 6.4 mmol) and DMAP (5 mg). Then the reaction mixture was heated to 111° C. for 16 h (note: nitrogen gas needs to be released). The reaction mixture was cooled down to r.t and concentrated in vacuo, purified by Teledyne ISCO CombiFlash to give a mixture of two isomers. The mixture was further separated by chiral SFC (CHIRALPAK IG, 30 mm×250 mm, 5 um column, eluting with 20% methanol in CO₂, at flow rate of 100 mL/min over 6 min). Retention time was 3.5 min for peak 1 and 4.7 min for peak 2. The desired isomer was collected as the second peak at 4.7 min as a single compound. LCMS calculated for C₁₆H₂₁FNO₄ (M+H)⁺: m/z=310.1; found: 310.1.

### Step 3. Isopropyl 1-amino-3-fluorocyclobutane-1-carboxylate

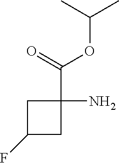

To a vial containing isopropyl 1-(((benzyloxy)carbonyl)amino)-3-fluorocyclobutane-1-carboxylate (1.52 g, 4.9 mmol, Step 2) in MeOH (20 mL) was added Pd/C (30 mg, 10% w/w). The vial was purged with hydrogen gas for 5 min and then stirred for 16 h under an atmosphere of hydrogen at 40° C. After cooling to room temperature, the reaction mixture was filtered through Celite and washed with CH₂Cl₂, followed by concentration of the filtrate in vacuo. The crude product was used directly in the next step without further purification. LCMS calculated for C₈H₁₅FNO₂ (M+H)⁺: m/z=176.1; found: 176.1.

### Step 4. Isopropyl 1-((6-bromo-3-cyanopyridin-2-yl)amino)-3-fluorocyclobutane-1-carboxylate

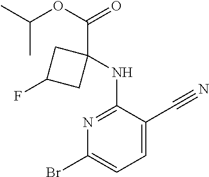 [SMILES: CC(C)OC(=O)C1(Nc2nc(Br)ccc2C#N)CC(F)C1]

To a vial containing 6-bromo-2-fluoronicotinonitrile (500 mg, 2.49 mmol) and isopropyl 1-amino-3-fluorocyclobutane-1-carboxylate (440 mg, 2.49 mmol) in DMSO (830 μL) was added DIPEA (520 μL, 3.00 mmol). After heating at 65° C. for 16 h, the reaction mixture was directly purified by Biotage Isolera to give the desired product as light-yellow solid. LCMS calculated for C₁₄H₁₆BrFN₃O₂ (M+H)⁺: m/z=356.0/358.0; found 356.0/358.0.

### Step 5. Isopropyl 1-(3-amino-6-bromo-1H-pyrazolo[3,4-b]pyridin-1-yl)-3-fluorocyclobutane-1-carboxylate

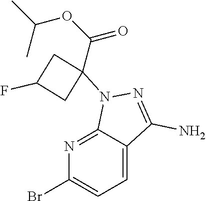 [SMILES: CC(C)OC(=O)C1(n2nc(N)c3ccc(Br)nc32)CC(F)C1]

To a vial containing isopropyl 1-((6-bromo-3-cyanopyridin-2-yl)amino)-3-fluorocyclobutane-1-carboxylate (750 mg, 2.11 mmol) in THF (2.8 mL) and DMF (5.6 mL) at 0° C. was slowly added LHMDS (2.7 mL, 2.74 mmol, 1.0 M in THF). After stirring at 0° C. for 10 min, (aminooxy)diphenylphosphine oxide (590 mg, 2.53 mmol) was added to the reaction mixture. The reaction was allowed to warm up to r.t. with monitoring of stirring rate. After stirring at r.t. for 1 h, the reaction mixture was treated with 1 M HCl solution (5 mL) and stirred for 10 min. The reaction mixture was neutralized with sat. NaHCO₃ solution, followed by extraction with diethyl ether (15 mL) for 3 times. The organic phase was combined and washed with brine, dried over MgSO₄ and concentrated in vacuo. The crude material was purified by Biotage Isolera to give the desired product as light-yellow solid. LCMS calculated for C₁₄H₁₇BrFN₄O₂ (M+H)⁺: m/z=371.0/373.0; found 371.1/373.0.

### Step 6 Isopropyl 1-(6-bromo-1H-pyrazolo[3,4-b]pyridin-1-yl)-3-fluorocyclobutane-1-carboxylate

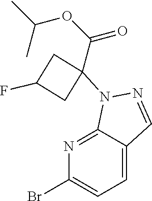 [SMILES: CC(C)OC(=O)C1(n2ncc3ccc(Br)nc32)CC(F)C1]

To a vial containing isopropyl 1-(3-amino-6-bromo-1H-pyrazolo[3,4-b]pyridin-1-yl)-3-fluorocyclobutane-1-carboxylate (700 mg, 1.89 mmol) in THF (2.8 mL) was slowly added tert-butyl nitrite (1.11 mL, 8.42 mmol). After heating at 65° C. for 1.5 h, the reaction mixture was concentrated in vacuo. The crude material was purified by Biotage Isolera to give the desired product as light-yellow solid. LCMS calculated for C₁₄H₁₆BrFN₃O₂ (M+H)⁺: m/z=356.0/358.0; found 356.0/358.0.

### Step 7. Isopropyl 3-fluoro-1-(6-((1-((1R,3R)-3-((methoxycarbonyl)amino)cyclopentyl-3-d)-3-(methyl-d)-2-oxo-2,3-dihydro-1H-imidazo[4,5-c]pyridin-6-yl)amino)-1H-pyrazolo[3,4-b]pyridin-1-yl)cyclobutane-1-carboxylate

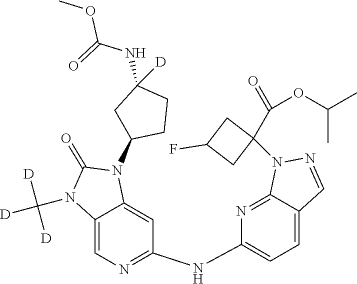 [SMILES: [2H]C([2H])([2H])n1c(=O)n([C@@H]2CC[C@@]([2H])(NC(=O)OC)C2)c2cc(Nc3ccc4cnn(C5(C(=O)OC(C)C)CC(F)C5)c4n3)ncc21]

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (33 mg, 0.11 mmol, Intermediate A), isopropyl 1-(6-bromo-1H-pyrazolo[3,4-b]pyridin-1-yl)-3-fluorocyclobutane-1-carboxylate (38 mg, 0.11 mmol), tris(dibenzylideneacetone)-dipalladium(0) (12 mg, 0.012 mmol), 4,5-bis(diphenylphosphino)-9,9-dimethylxanthene (15 mg, 0.025 mmol), and cesium carbonate (70 mg, 0.21 mmol) was evacuated and backfilled with nitrogen three times, followed by the addition of 1,4-dioxane (0.5 mL). The vial was sealed and heated to 100° C. for 2 h. After cooling to r.t., the mixture was filtered and concentrated in vacuo. The crude material was purified by Biotage Isolera to give the desired product as off-white solid. LCMS calculated for C₂₈H₃₀D₄FN₈O₅ (M+H)⁺: m/z=585.3; found 585.3.

### Step 8. Methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate

To a vial containing isopropyl 3-fluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (20 mg, 0.034 mmol) in THF/MeOH/water (1.5 mL, v/v/v=1/1/1) was added LiOH (7 mg, 0.3 mmol). After heating at 50° C. for 15 min, the reaction mixture was cooled to r.t. and diluted with CH₂Cl₂ (5 mL). The pH of the mixture was adjusted to ~4 using HCl aqueous solution, followed by extraction with CH₂Cl₂ (3×5 mL). The organic layers were combined, dried over MgSO₄ and concentrated in vacuo. To a vial containing the obtained crude acid was added thionyl chloride (1.0 mL). The reaction mixture was heated to 50° C. for 30 min. After cooling to r.t., the reaction mixture was concentrated in vacuo to remove thionyl chloride. The crude acid chloride was dissolved in THF (2.0 mL) and added dropwise to the stirring ammonia aqueous solution (5.0 mL) at 0° C. After the addition of the acid chloride solution, the reaction mixture was extracted into CH₂Cl₂. The organic layers were combined, dried over MgSO₄ and concentrated in vacuo. The crude product was purified by prep-LCMS (XBridge C18 column, eluting with a gradient of acetonitrile/water containing 0.1% TFA, at flow rate of 60 mL/min). The final product was obtained as a single diastereomer. LCMS calculated for C₂₅H₂₅D₄N₉O₄ (M+H)⁺: m/z=542.2; found 542.2. ¹H NMR (600 MHz, DMSO) δ 11.42 (brs, 1H), 8.27 (s, 1H), 8.25 (d, J=9.0 Hz, 1H), 8.19 (s, 1H), 7.58 (s, 1H), 7.53 (brs, 1H), 7.34 (s, 1H), 7.28 (brs, 1H), 7.02 (d, J=8.7 Hz, 1H), 5.32-5.14 (m, 1H), 5.01 (p, J=8.5 Hz, 1H), 3.55 (s, 3H), 3.47-3.40 (m, 2H), 3.12-3.02 (m, 2H), 2.34 (dd, J=13.6, 7.5 Hz, 1H), 2.25-2.13 (m, 2H), 2.10-2.02 (m, 1H), 1.97 (dd, J=13.6, 9.6 Hz, 1H), 1.66 (dt, J=12.4, 8.0 Hz, 1H).

### Example B-25. Methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate and Methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate (Compound 1)

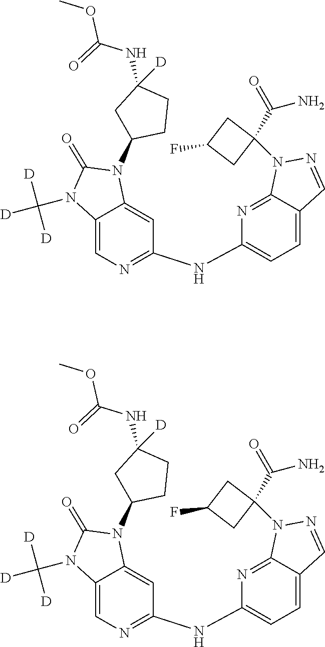 [SMILES: [2H]C([2H])([2H])n1c(=O)n([C@@H]2CC[C@@]([2H])(NC(=O)OC)C2)c2cc(Nc3ccc4cnn([C@]5(C(N)=O)C[C@@H](F)C5)c4n3)ncc21 ; [2H]C([2H])([2H])n1c(=O)n([C@@H]2CC[C@@]([2H])(NC(=O)OC)C2)c2cc(Nc3ccc4cnn([C@]5(C(N)=O)C[C@H](F)C5)c4n3)ncc21]

### Step 1. Methyl 1-((6-bromo-3-cyanopyridin-2-yl)amino)-3-fluorocyclobutane-1-carboxylate

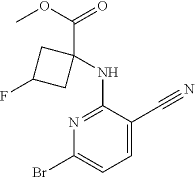 [SMILES: COC(=O)C1(Nc2nc(Br)ccc2C#N)CC(F)C1]

To a vial containing 6-bromo-2-fluoronicotinonitrile (300 mg, 1.49 mmol) and methyl 1-amino-3-fluorocyclobutane-1-carboxylate (242 mg, 1.64 mmol) in DMSO (500 μL) was added DIPEA (340 μL, 1.94 mmol). After heating at 65° C. for 16 h, the reaction mixture was directly purified by Biotage Isolera to give the desired product as light-yellow solid. LCMS calculated for C₁₂H₁₂BrFN₃O₂ (M+H)⁺: m/z=328.0/330.0; found 328.0/330.0.

### Step 2. Methyl (1r, 3r)-1-(3-amino-6-bromo-1H-pyrazolo[3,4-b]pyridin-1-yl)-3-fluorocyclobutane-1-carboxylate and Methyl (1s,3s)-1-(3-amino-6-bromo-1H-pyrazolo[3,4-b]pyridin-1-yl)-3-fluorocyclobutane-1-carboxylate

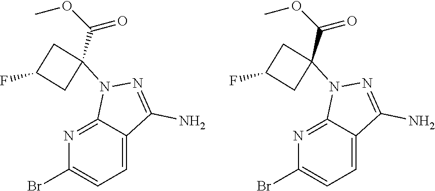 [SMILES: COC(=O)[C@]1(n2nc(N)c3ccc(Br)nc32)C[C@H](F)C1 ; COC(=O)[C@]1(n2nc(N)c3ccc(Br)nc32)C[C@@H](F)C1]

To a vial containing methyl 1-((6-bromo-3-cyanopyridin-2-yl)amino)-3-fluorocyclobutane-1-carboxylate (100 mg, 0.30 mmol) in THF (0.4 mL) and DMF (0.8 mL) at 0° C. was slowly added LHMDS (0.40 mL, 0.40 mmol, 1.0 M in THF). After stirring at 0° C. for 10 min, (aminooxy)diphenylphosphine oxide (71 mg, 0.30 mmol) was added to the reaction mixture. The reaction was allowed to warm up to r.t. with monitoring of stirring rate. After stirring at r.t. for 1 h, the reaction mixture was treated with 1 M HCl solution (2 mL) and stirred for 10 min. The reaction mixture was neutralized with sat. NaHCO₃ solution, followed by extraction with diethyl ether (5 mL) for 3 times. The organic phase was combined and washed with brine, dried over MgSO₄ and concentrated in vacuo. The crude material was purified by Biotage Isolera to give the title compound as a mixture of two diastereomers. The mixture was further separated by chiral SFC (Phenomenex-Amylose-1, 30 mm×250 mm, 5 um column, eluting with 20% methanol in CO₂, at flow rate of 125 mL/min over 5.3 min). The first peak (Peak 1) was collected at 3.1 min as a single diastereomer. The second peak (Peak 2) was collected at 3.42 min as a single diastereomer. Both peaks were individually carried forward through next steps.

Peak 1: LCMS calculated for C₁₂H₁₃BrFN₄O₂ (M+H)⁺: m/z=343.0/345.0; found: 343.0/345.0.

Peak 2: LCMS calculated for C₁₂H₁₂BrFN₄O₂ (M+H)⁺: m/z=343.0/345.0; found 343.1/345.0.

### Step 3. Methyl (1r,3r)-1-(6-bromo-1H-pyrazolo[3,4-b]pyridin-1-yl)-3-fluorocyclobutane-1-carboxylate and Methyl (1s,3s)-1-(6-bromo-1H-pyrazolo[3,4-b]pyridin-1-yl)-3-fluorocyclobutane-1-carboxylate

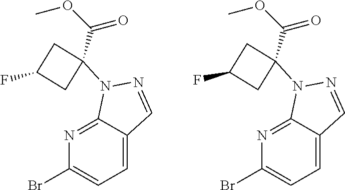 [SMILES: COC(=O)[C@]1(n2ncc3ccc(Br)nc32)C[C@H](F)C1 ; COC(=O)[C@]1(n2ncc3ccc(Br)nc32)C[C@@H](F)C1]

To a vial containing methyl 1-(3-amino-6-bromo-1H-pyrazolo[3,4-b]pyridin-1-yl)-3-fluorocyclobutane-1-carboxylate (40 mg, 0.12 mmol, isomer of Peak 1 from Step 2) in THF (0.4 mL) was slowly added tert-butyl nitrite (70 μL, 0.5 mmol). After heating at 65° C. for 1.5 h, the reaction mixture was concentrated in vacuo. The crude material was purified by Biotage Isolera to give the desired product (Intermediate 25-3-1) as light-yellow solid. LCMS calculated for C₁₂H₁₂BrFN₃O₂ (M+H)⁺: m/z=328.0/330.0; found 328.1/330.0. The other isomer (Intermediate 25-3-2) was also prepared in the same manner starting with the isomer of Peak 2 in Step 2. LCMS calculated for C₁₂H₁₂BrFN₃O₂ (M+H)⁺: m/z=328.0/330.0; found 328.0/330.0 (for both Intermediates 25-3-1 and 25-3-2).

### Step 4. Methyl (1r, 3R)-3-fluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate and Methyl (is, 3S)-3-fluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate

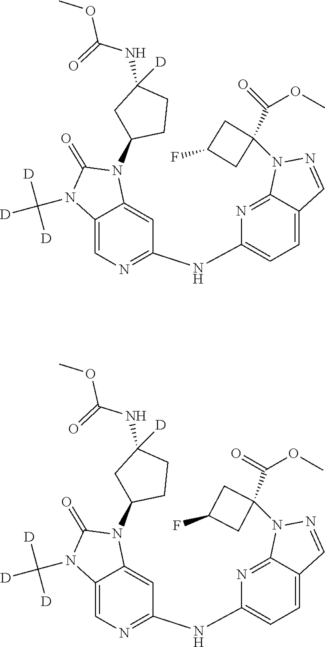 [SMILES: [2H]C([2H])([2H])n1c(=O)n([C@@H]2CC[C@@]([2H])(NC(=O)OC)C2)c2cc(Nc3ccc4cnn([C@]5(C(=O)OC)C[C@@H](F)C5)c4n3)ncc21 ; [2H]C([2H])([2H])n1c(=O)n([C@@H]2CC[C@@]([2H])(NC(=O)OC)C2)c2cc(Nc3ccc4cnn([C@]5(C(=O)OC)C[C@H](F)C5)c4n3)ncc21]

A vial containing methyl ((1R,3R)-3-(6-amino-3-(methyl-d₃)-2-oxo-2,3-dihydro-1H-imidazo[4,5-c]pyridin-1-yl)cyclopentyl-1-d)carbamate (28 mg, 0.09 mmol, Intermediate A), methyl 1-(6-bromo-1H-pyrazolo[3,4-b]pyridin-1-yl)-3-fluorocyclobutane-1-carboxylate (30 mg, 0.09 mmol, Intermediate 25-3-1 of Step 3), tris(dibenzylideneacetone)-dipalladium(0) (9.0 mg, 0.010 mmol), 4,5-bis(diphenylphosphino)-9,9-dimethylxanthene (11.6 mg, 0.020 mmol), and cesium carbonate (60 mg, 0.18 mmol) was evacuated and backfilled with nitrogen three times, followed by the addition of 1,4-dioxane (0.5 mL). The vial was sealed and heated to 100° C. for 2 h. After cooling to r.t., the mixture was filtered and concentrated in vacuo. The crude material was purified by Biotage Isolera to give the desired product (Intermediate 25-4-1) as off-white solid. LCMS calculated for C₂₆H₂₆D₄FN₈O₅ (M+H)⁺: m/z=557.3; found 557.3. The other isomer was also prepared in the same manner starting with Intermediate 25-3-2 of Step 3. LCMS calculated for C₂₆H₂₆D₄FN₈O₅ (M+H)⁺: m/z=557.3; found 557.3.

### Step 5. Methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate and Methyl ((1R,3R)-3-(6-((1-((1s, 3S)-1-carbamoyl-3-fluorocyclobutyl)-1H-pyrazolo[3,4-b]pyridin-6-yl)amino)-3-(methyl-d₃)-2-oxo-2,3-dihydro-H-imidazo[4,5-c]pyridin-1-yl)cyclopentyl-1-d)carbamate

To a vial containing methyl 3-fluoro-1-(6-((1-((1R,3R)-3-((methoxycarbonyl)amino)cyclopentyl-3-d)-3-(methyl-d₃)-2-oxo-2,3-dihydro-1H-imidazo[4,5-c]pyridin-6-yl)amino)-1H-pyrazolo[3,4-b]pyridin-1-yl)cyclobutane-1-carboxylate (20 mg, 0.036 mmol, Intermediate 25-4-1 of Step 4) in THF/MeOH/water (1.5 mL, v/v/v=1/1/1) was added LiOH (7 mg, 0.3 mmol). After heating at 50° C. for 15 min, the reaction mixture was cooled to r.t. and diluted with CH₂Cl₂ (5 mL). The pH of the mixture was adjusted to ~4 using HCl aqueous solution, followed by extraction with CH₂Cl₂ (3×5 mL). The organic layers were combined, dried over MgSO₄ and concentrated in vacuo. To a vial containing the obtained crude acid was added thionyl chloride (1.0 mL). The reaction mixture was heated to 50° C. for 30 min. After cooling to r.t., the reaction mixture was concentrated in vacuo to remove thionyl chloride. The crude acid chloride was dissolved in THF (2.0 mL) and added dropwise to the stirring ammonia aqueous solution (5.0 mL) at 0° C. After the addition of the acid chloride solution, the reaction mixture was extracted into CH₂Cl₂. The organic layers were combined, dried over MgSO₄ and concentrated in vacuo. The crude product was purified by prep-LCMS (XBridge C18 column, eluting with a gradient of acetonitrile/water containing 0.10% TFA, at flow rate of 60 mL/min) resulting in single isomer Product 1. The other isomer (Product 2) was also prepared in the same manner starting with Intermediate 25-4-2 of Step 4.

Product 1: LCMS calculated for C₂₅H₂₅D₄FN₉O₄ (M+H)⁺: m/z=542.3; found 542.3.

Product 2: LCMS calculated for C₂₅H₂₅D₄FN₉O₄ (M+H)⁺: m/z=542.3; found 542.3.

### Example B-26. Methyl ((1R,3R)-3-(6-((7-((1s,3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate and Methyl ((1R,3R)-3-(6-((7-((1r,3R)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate

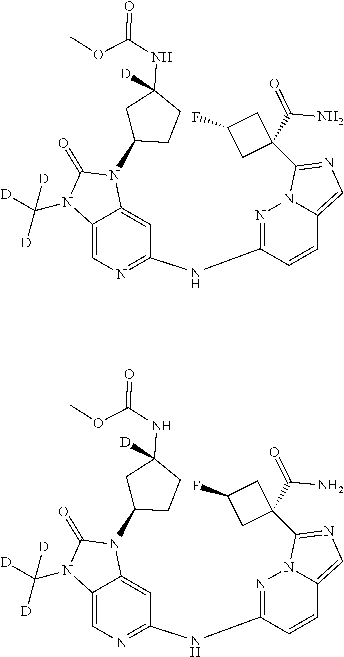 [SMILES: [2H]C([2H])([2H])n1c(=O)n([C@@H]2CC[C@@]([2H])(NC(=O)OC)C2)c2cc(Nc3ccc4cnc([C@]5(C(N)=O)C[C@@H](F)C5)n4n3)ncc21 ; [2H]C([2H])([2H])n1c(=O)n([C@@H]2CC[C@@]([2H])(NC(=O)OC)C2)c2cc(Nc3ccc4cnc([C@]5(C(N)=O)C[C@H](F)C5)n4n3)ncc21]

### Step 1. Isopropyl (is, 3s)-1-(2-chloroimidazo[1,5-b]pyridazin-7-yl)-3-fluorocyclobutane-1-carboxylate-(2-chloroimidazo[1,5-b]pyridazin-7-yl)-3-fluorocyclobutane-1-carboxylate and Isopropyl (1r, 3r)-1-(2-chloroimidazo[1,5-b]pyridazin-7-yl)-3-fluorocyclobutane-1-carboxylate

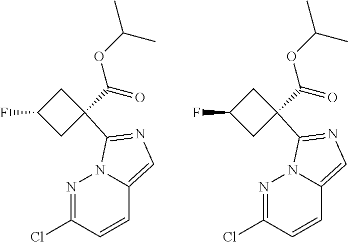 [SMILES: CC(C)OC(=O)[C@]1(c2ncc3ccc(Cl)nn32)C[C@H](F)C1 ; CC(C)OC(=O)[C@]1(c2ncc3ccc(Cl)nn32)C[C@@H](F)C1]

The title compound was prepared according to the procedures described in Example B-5, Steps 1-2. After SFC chiral purification, the compounds of both peaks were individually carried forward through next steps. Retention time was 3.5 min for Peak 1 and 4.9 min for Peak 2.

Peak 1: LCMS calculated for C₁₄H₁₆ClFN₃O₂ (M+H)⁺: m/z=312.1; found 312.1.

Peak 2: LCMS calculated for C₁₄H₁₆ClFN₃O₂ (M+H)⁺: m/z=312.1; found 312.1.

### Step 2. Methyl ((1R,3R)-3-(6-((7-((1s, 3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate and Methyl ((1R,3R)-3-(6-((7-((1r,3R)-1-carbamoyl-3-fluorocyclobutyl)imidazo[1,5-b]pyridazin-2-yl)amino)-3-(methyl-d)-2-oxo-2,3-dihydro-1H-imidazo[4,5-c]pyridin-1-yl)cyclopentyl-1-d)carbamate

The title compounds were prepared according to the procedures described in Example B-5, Steps 3-5, with isopropyl 1-(2-chloroimidazo[1,5-b]pyridazin-7-yl)-3-fluorocyclobutane-1-carboxylate (peak 1 and peak 2 individually) replacing isopropyl 1-(2-chloroimidazo[1,5-b]pyridazin-7-yl)-3-fluorocyclobutane-1-carboxylate in Step 3. Each desired product (Product 1 obtained using isomer of Peak 1 in Step 1 and Product 2 obtained using isomer of Peak 2 in Step 1) was obtained as a single diastereomer.

Product 1: LCMS calculated for C₂₅H₂₅D₄FN₉O₄ (M+H)⁺: m/z=542.3; found 542.3.

Product 2: LCMS calculated for C₂₅H₂₅D₄FN₉O₄ (M+H)⁺: m/z=542.3; found 542.3.

### Example 1. In Vitro VF Kinase Assay

The JAK inhibitors of the present disclosure, which can be used for the treatment of clonal hematopoietic disorders, have been tested for inhibitory activity according to the following in vitro assay.

JAK2 JH2-V617F binding assay utilizes pseudo-kinase domain (JH2, amino-acids 536-812 with 3 surface mutations W659A, W777A, F794H) of human V617F mutant JAK2 expressed as C-terminal His-Avi-tagged, biotinylated protein in a baculovirus expression system (BPS Bioscience, Catalog #79498). The assay was conducted in black 384-well polystyrene plates in a final reaction volume of 20 μL. JAK2 JH2-V617F (0.26 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

The compounds in Table 1 were tested in this assay and shown to have the IC₅₀ values in Table A.

TABLE A

| Example | JH2 BIND V617F |
|---|---|
| A-1 | + |
| A-2 | + |
| A-3 | + |

+ refers to IC₅₀ of ≤10 nM

### Example 2. Biochemical Affinity Assay

The biochemical affinity of the compounds was measured using a TR-FRET assay in a 384-well plate, in which biotinylated JAK2-JH2 domain (for both wild type and the V617F mutant) and compounds of interest at 11 serially diluted concentrations were mixed together in the assay buffer that contains 50 mM Tris, 10 mM MgCl₂, 0.01% Brij-35, 0.1% BSA, 1 mM EGTA, 5 mM DTT, 5% glycerol, 0.5% DMSO, at pH 7.5. The reaction was incubated at room temperature to allow to reach equilibrium, followed by addition of the detection solution that contains Tb cryptate-labeled Streptavidin and a fluorescein-labelled tracer molecule. The plate was then read by BMG PHERAstar using LanthaScreen module.

Data was analyzed using the Genedata platform. The IC₅₀ values of non-tight binding compounds were calculated using Equation 1.

$$\frac{{v}_{i}}{{v}_{0}} = \frac{1}{1 + ( \frac{[ I ]}{{IC}_{50}} )} . \qquad Equation 1$$

For tight binding compounds, K_{i,}^{app} values were calculated using the quadratic Morrison equation, where the enzyme concentration was also taken into consideration, as shown in Equation 2.

$$\frac{{v}_{i}}{{v}_{0}} = 1 - \frac{\begin{matrix}( {[ E ]}_{T} + {[ I ]}_{T} + {K}_{i}^{a p p} ) - \\ \sqrt{{( {[ E ]}_{T} + {[ I ]}_{T} + {K}_{i}^{app} )}^{2}} - {4 [ E ]}_{T} )\end{matrix}}{( {2 [ E ]}_{T} )} . \qquad Equation 2$$

TABLE B

| Example | JH2 BIND V617F Ki (nM) |
|---|---|
| B-1 | ** |
| B-2 | * |
| B-3 | * |
| B-4 | ** |
| B-5 | * |
| B-6 | * |
| B-7 | * |
| B-8 | * |
| B-9 | * |
| B-10 | * |
| B-11 | * |
| B-12 | * |
| B-13 | * |
| B-14 | * |
| B-15 | * |
| B-16 | * |
| B-17 | * |
| B-18 | * |
| B-19 | * |
| B-20 | * |
| B-21 | * |
| B-22 | * |
| B-23 | ** |
| B-24 | * |
| B-25, Product 1 | * |
| B-25, Product 2 | ** |
| B-26, Product 1 | * |
| B-26, Product 2 | ** |

* refers to IC₅₀ of ≤0.1 nM
** refers to IC₅₀ of >0.1 nM to ≤1 nM

### Example C. In Vivo Models

Clonal hematopoiesis correlates with increased likelihood of developing several more severe diseases including hematologic malignancies such as MPN and AML, and cardiovascular diseases (CVD) such as atherosclerosis and venous thrombosis (see e.g., Weeks et al., NEJM Evid., 2023 May; Jaiswal et al., N. Engl. J. Med. 2017; Wolach et al., Sci. Transl. Med. 2018; Wang et al., Circ. Res. 2018 Nov. 9; 123(11)).

In vivo models consisting of mice inoculated with hematopoietic cells (human or murine) expressing the JAK2V617F oncogenic mutation (to establish clonal hematopoiesis) are used to demonstrate efficacy of JAK2V617F selective inhibitors against clonal hematopoiesis or its subsequent effects (e.g., MPN, CVD, etc.). A JAK2V617F selective inhibitor decreases the numbers of JAK2V617F expressing cells (allele burden) in mice and decreases the effects of these cells including aberrant cytokine production, inflammation, and clotting ability/Netosis. JAK2V617F allelic content is measurable by quantitative PCR methods. Inflammatory cytokine analyses are performed using Luminex, MSD, or OLink platforms. Netosis as a surrogate for clotting, is quantified ex vivo via commercially available citrullinated H₃ ELISA kits, or by flow cytometry focused on neutrophils positively stained for DAPI and Sytox Orange. Clotting is also monitored by following megakaryocyte and platelet number, maturity, and activation, as well as by mouse tail vein coagulation assays.

Mice with mutations in enzymes regulating lipid clearance from blood (strains B6.129P2-Apoetm1Unc/J and B6.129S7-Ldlrtm1Her/J; Jackson Labs) are also used as recipients for JAK2V617F expressing cells, as models for atherosclerosis with concurrent clonal hematopoiesis. In these models, JAK2V617F selective inhibitor efficacy is monitored not only by decreased numbers of JAK2V617F cells, but also by decreased numbers of atherosclerotic plaques or macrophages found therein. Size and quantity of atherosclerotic plaques is determined by histologic staining analyses of the mouse aortic root such as hematoxylin and eosin to determine sizes of plaques, Oil Red O to determine fat content, or Sirius Red to determine collagen/fibrotic content. Likewise infiltrating inflammatory cells such as macrophages are quantified as well. JAK2V617F selective inhibition decreases the number and sizes of atherosclerotic plaques in mice bearing JAK2V617F hematopoietic cells and decreases the number of hyperinflammatory JAK2V617F⁺ macrophages found in these aortic lesions.

### Example D. Cardiovascular Disease-Inducing Neutrophil Extracellular Traps are Suppressed by a JAK2V617F Selective Inhibitor in a Mouse Model of Clonal Hematopoiesis of Indeterminate Potential (CHIP)

JAK2V617F-driven CHIP has been linked to increased findings of neutrophil extracellular traps (NETs), and the subsequent death of those neutrophils termed NETosis (see e.g., Wolach et al, Sci. Transl. Med. 10, eaan8292 (2018)). Formation of NETs is a common function of neutrophils. Neutrophils will extrude their decompacted DNA strands, forming an adherent “net” that can capture pathogens (see e.g., Meier et al., J. Inf. Disease, 2024, 230, 514-521). However, aberrant NET production, such as is the case in CHIP/MPN has been linked to cardiovascular events. NETs and NETosis have been shown to have a role in inducing thrombosis as well as in progression of atherosclerosis (see e.g., Molinaro et al, Int. J Cardiol., 2024, 15, 132184).

NETosis was induced by injecting JAK2V617F-expressing CD34⁺ cells from a myelofibrosis patient into female NSG-S mice (1×10⁶ cells injected intratibially). Four weeks post injection, engraftment was verified by detecting human CD45⁺ cells from blood by flow cytometry. Mice were then changed to a “Western” diet. This diet was higher in cholesterol than standard mouse chow and drives mice toward a more inflammatory state mimicking that of a patient with cardiovascular disease. After 6 weeks on the Western diet, NETosis was visualized by flow cytometry from mouse peripheral blood. NETosis was defined as transplanted human neutrophils (identified as hCD45⁺, CD14⁻, CD16⁺) that also stained positively for citrullinated histone H₃ (H3Cit⁺-the marker of neutrophil extracellular chromatin).

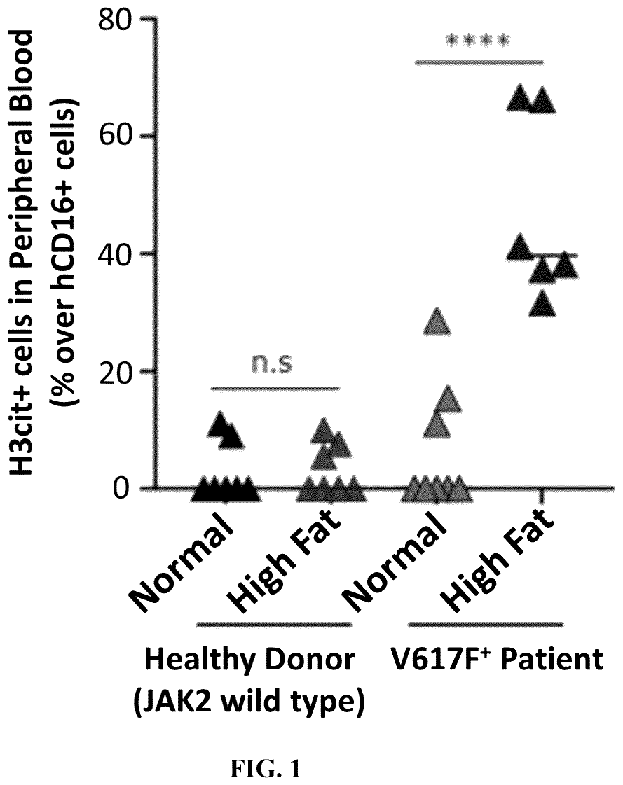

As shown in FIG. 1, NETosis was present in mice that harbor JAK2V617F-expressing neutrophils, and significantly more NETosis is found in mice with JAK2V617F neutrophils and that have been fed Western diet. Mice harboring cells from healthy donors on a normal diet showed little NETosis and had an indistinguishable amount of NETosis when compared to mice on the high fat diet (FIG. 1: ns—non significant). Mice with JAK2V617F⁺ patient cells on a regular diet had an increase in NETosis. The mice with both JAK2V617F⁺ patient cells and the high fat diet had a statistically significant (****p<0.0001 versus mice on normal diet) increase in the percentage of neutrophils undergoing NETosis.

Mice were treated with the JAK2V617F selective inhibitor methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate (Compound 1) either to prevent or to reverse NET formation in mice. As above, JAK2V617F-expressing CD34⁺ cells from a myelofibrosis patient were injected into female NSG-S mice (1×10⁶ cells injected intratibially). Four weeks post injection, engraftment was verified by detecting human CD45⁺ cells from blood by flow cytometry. Mice were then changed to the “Western” high fat diet. At this point, one group of mice were dosed with Compound 1 at either 10 mg/kg BID or 30 mg/kg BID or vehicle control for 6 weeks to evaluate prevention of NETosis by a JAK2V617F inhibitor. Another group of these mice was allowed to remain untreated for four weeks, followed by 2 weeks of treatment with Compound 1 (10 mg/kg or 30 mg/kg BID) or vehicle control to test the effectiveness of a JAK2V617F inhibitor after NETosis has been established.

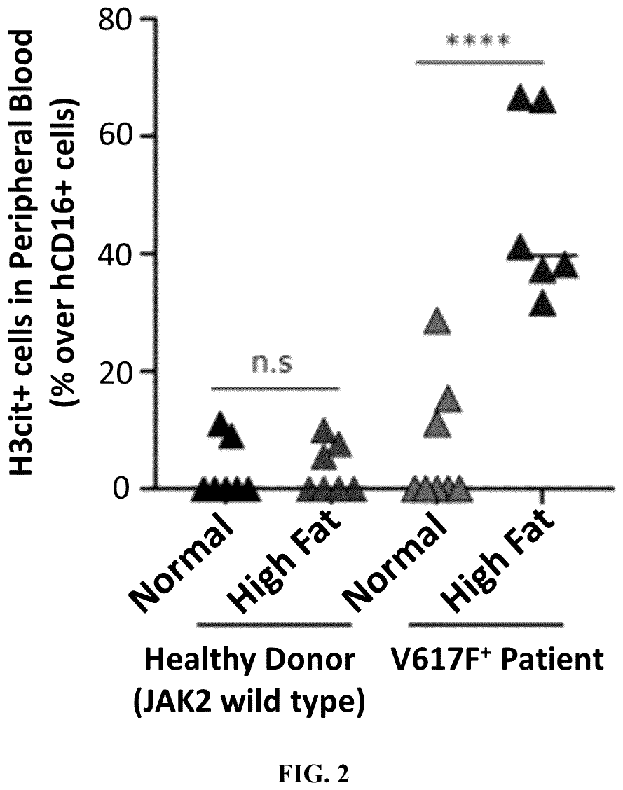

As shown in FIG. 2, mice that were administered Compound 1 had significantly (****p<0.0001 versus vehicle treated mice) reduced levels of NETosis compared to mice receiving vehicle control.

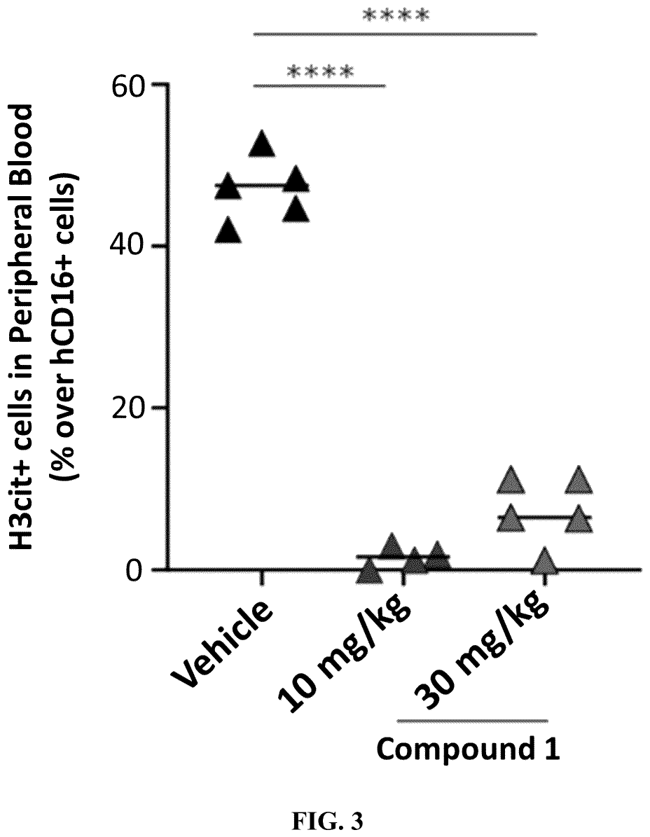

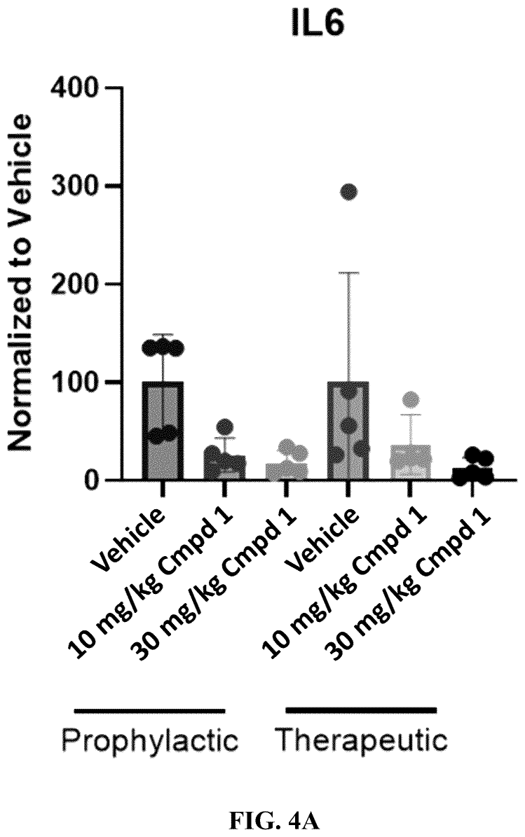

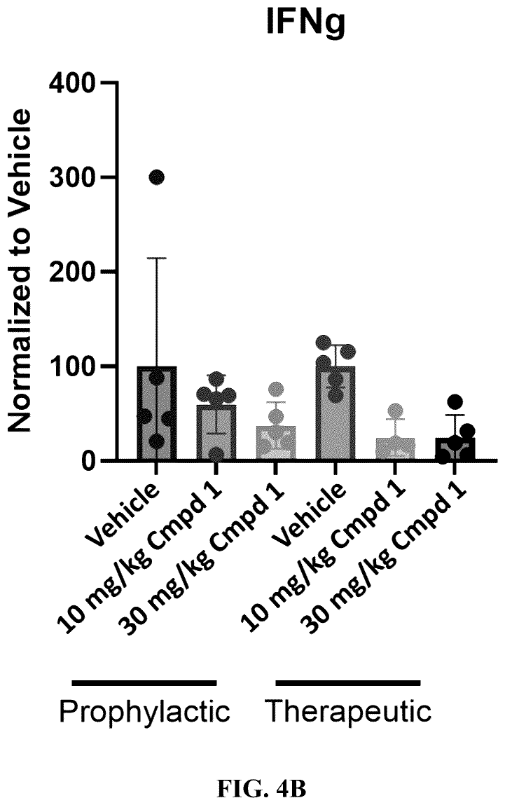

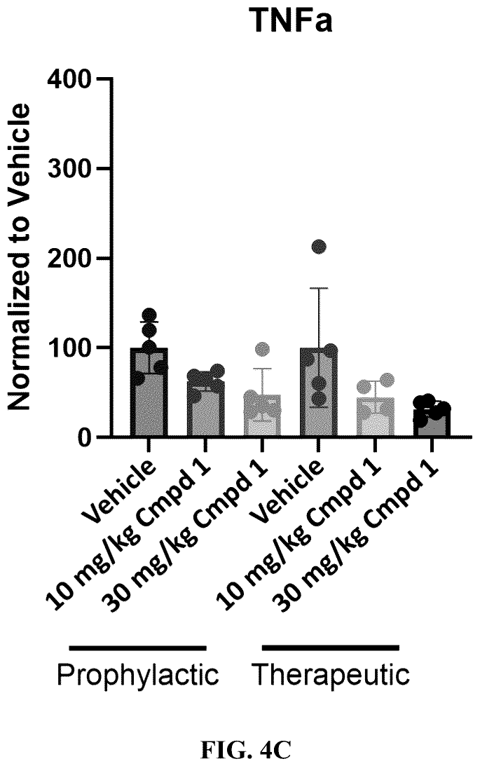

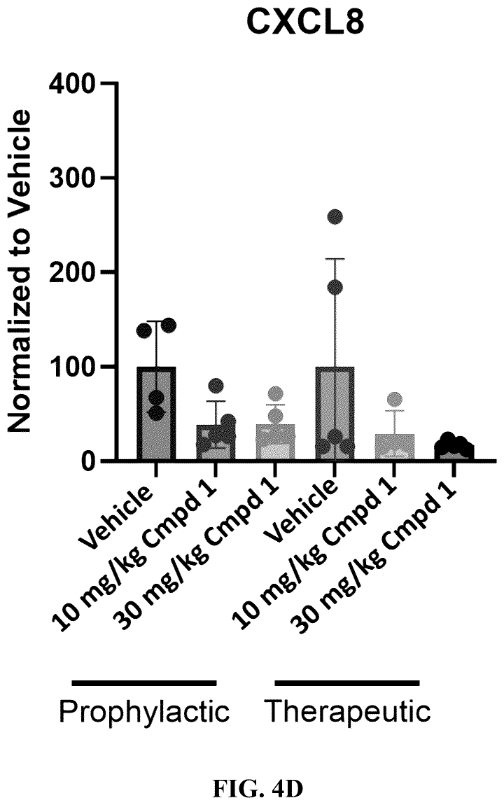

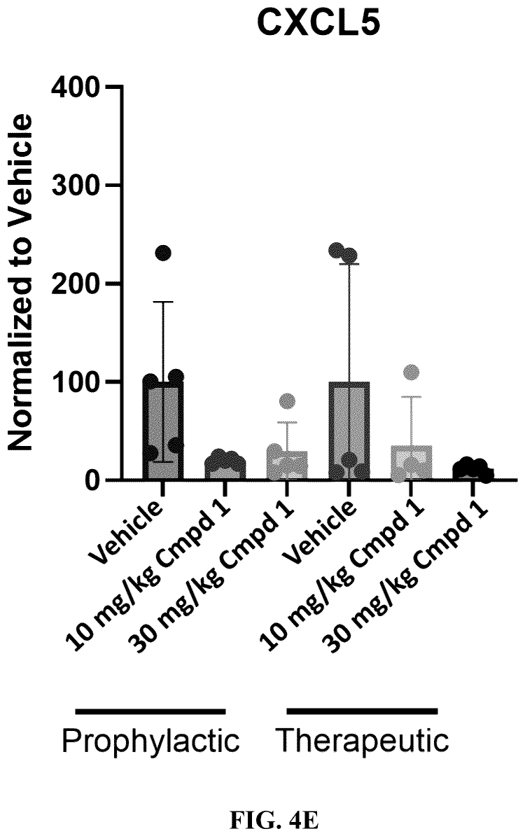

Likewise, FIG. 3 shows that Compound 1 also significantly (****p<0.0001 versus vehicle treated mice) reduced levels of NETosis in mice that were not administered Compound 1 until after NETosis is detectable in the mice. In addition, plasma was isolated from these mice at termination of the study to analyze levels of neutrophil activating cytokines. FIGS. 4A-4E show that JAK2V617F selective inhibition via Compound 1 reduced the levels of inflammatory cytokines linked to neutrophil activity.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating clonal hematopoiesis of indeterminate potential in a patient in need thereof, comprising administering to the patient a therapeutically effective amount of a compound selected from:
methyl ((1R,3R)-3-(7-(3-fluoro-1-(methyl-d₃)-1H-pyrazol-4-yl)-3-(methyl-d₃)-8-(1-(methyl-d₃)-1H-indazol-5-yl)-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate;
methyl ((1R,3R)-3-(8-(1,1-dimethyl-1,3-dihydroisobenzofuran-5-yl)-7-(3-fluoro-1-(methyl-d₃)-1H-pyrazol-4-yl)-3-(methyl-d₃)-2-oxo-3,6-dihydroimidazo[4,5-d]pyrrolo[2,3-b]pyridin-1(2H)-yl)cyclopentyl)carbamate;
methyl ((3R)-3-(6-((1-(1-carbamoylcyclopropyl)-4-(tetrahydro-2H-pyran-4-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-4-cyclopropyl-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-2-methylcyclopropyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclopropylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(tetrahydro-2H-pyran-4-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-cyclobutylimidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)imidazo[1,5-b]pyridazin-2-yl-5-d)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-(1-carbamoyl-3-fluorocyclobutyl)-5-(2-hydroxypropan-2-yl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1,2,2,5,5-d₅)carbamate;
methyl ((1R,3R)-3-(6-((1-(1-carbamoylcyclobutyl)-4-(trans-4-hydroxycyclohexyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3,3-difluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-methoxycyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-2-methylcyclopropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(1-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxyethyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoylcyclobutyl)-3-(2-hydroxypropyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-3-(2-hydroxypropan-2-yl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-7-methyl-2-(trifluoromethyl)-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-carbamoyl-3-fluorocyclobutyl)-2-(difluoromethyl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((3-(1-amino-2-cyclopropyl-1-oxopropan-2-yl)-7-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-(1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1r,3R)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1s,3S)-1-carbamoyl-3-fluorocyclobutyl)-1H-pyrazolo[3,4-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((7-((1s,3S)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate; and
methyl ((1R,3R)-3-(6-((7-((1r,3R)-1-carbamoyl-3-fluorocyclobutyl)imidazo[1,5-b]pyridazin-2-yl)amino)-3-(methyl-d₃)-2-oxo-2,3-dihydro-1H-imidazo[4,5-c]pyridin-1-yl)cyclopentyl-1-d)carbamate;
or a pharmaceutically acceptable salt of any of the aforementioned.

2. The method of claim 1, wherein the compound is selected from:
methyl ((1R,3R)-3-(7-(3-fluoro-1-(methyl-d₃)-1H-pyrazol-4-yl)-3-(methyl-d₃)-8-(1-(methyl-d₃)-1H-indazol-5-yl)-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate; and
methyl ((1R,3R)-3-(8-(1,1-dimethyl-1,3-dihydroisobenzofuran-5-yl)-7-(3-fluoro-1-(methyl-d₃)-1H-pyrazol-4-yl)-3-(methyl-d₃)-2-oxo-3,6-dihydroimidazo[4,5-d]pyrrolo[2,3-b]pyridin-1(2H)-yl)cyclopentyl)carbamate;
or a pharmaceutically acceptable salt of any of the aforementioned.

3-4. (canceled)

5. The method of claim 1, wherein the compound is methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate, or a pharmaceutically acceptable salt thereof.

6. The method of claim 1, wherein the compound is methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate.

7-15. (canceled)

16. A method of treating a clonal hematopoietic disorder in a patient in need thereof, comprising administering to the patient a therapeutically effective amount of an antibody that binds to human mutant calreticulin (CALR).

17-33. (canceled)

34. A method of treating clonal hematopoiesis of indeterminate potential in a patient in need thereof, comprising administering to the patient a therapeutically effective amount of a compound of Formula I: 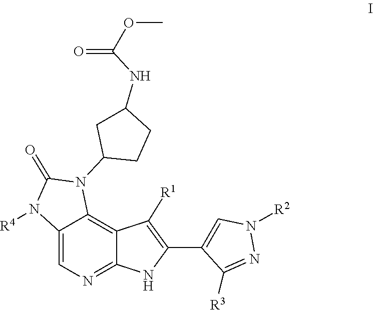
or a pharmaceutically acceptable salt thereof, wherein:
R¹ is selected from phenyl, indazolyl, and dihydroisobenzofuranyl, each of which is optionally substituted by 1, 2, or 3 substituents independently selected from halo, C₁₋₆ alkyl, C₁₋₆ alkoxy, C₁₋₆ haloalkyl, and C₁₋₆ hydroxyalkyl;
R² is C₁₋₆ alkyl or C₁₋₆ haloalkyl;
R³ is selected from halo, C₁₋₆ alkyl, and C₁₋₆ alkoxy; and
R⁴ is C₁₋₆ alkyl.

35. (canceled)

36. The method of claim 1, wherein the clonal hematopoiesis of indeterminate potential is selected from CALR CHIP, DNMT3A CHIP, TET2 CHIP, ASXL1 CHIP, SRSR2 CHIP, ZRSR2 CHIP, RUNX1 CHIP, IDH2 CHIP, SF₃B1 CHIP, JAK2 CHIP, JAK2 V617F CHIP, and non-JAK2 CHIP, or any combination thereof.

37. The method of claim 1, wherein the clonal hematopoiesis of indeterminate potential is JAK2 CHIP.

38. The method of claim 1, wherein the clonal hematopoiesis of indeterminate potential is JAK2 V617F CHIP.

39. The method of claim 1, wherein the clonal hematopoiesis of indeterminate potential is clonal cytopenia of undetermined significance (CCUS).

40. The method of claim 1, wherein the method further comprises treating a CHIP-induced disease in the patient.

41. The method of claim 1, wherein the method further comprises treating a JAK2 V617F CHIP-induced disease in the patient.

42. The method of claim 40, wherein the CHIP-induced disease is selected from hematologic cancer, myeloproliferative neoplasm, myeloid cancer, coronary heart disease, stroke, ischemic heart disease, venous thromboembolism, pulmonary embolism, drusen, age-related macular degeneration, chronic obstructive pulmonary disease (COPD), osteoporosis, chronic liver disease, acute kidney injury, thoracic aortic aneurysm, leukocytosis, neutropenia, hyperplasia, arteriosclerosis, phlebosclerosis caused by activated neutrophils, aortic aneurysm, hypertension, pulmonary hypertension, myeloid leukemia, lymphocytic leukemia, myelodysplastic syndrome, aplastic anemia, paroxysmal nocturnal hemoglobinuria, malignant lymphoma, and multiple myeloma.

43. The method of claim 40, wherein the method comprises treating CHIP-induced thrombosis in the patient.

44. The method of claim 43, wherein the CHIP-induced thrombosis comprises arterial thrombosis.

45. The method of claim 43, wherein the CHIP-induced thrombosis comprises venous thrombosis.

46. The method of claim 40, wherein the method comprises treating CHIP-induced atherosclerosis in the patient.

47. The method of a claim 40, wherein the CHIP-induced disease is selected from acute kidney injury, chronic liver disease, thoracic aortic aneurysm, coronary heart disease, and hematological malignancy.
